(19) United States
(12) Patent Application Publication
(10) Pub. No.: US 2022/0153859 A1
(43) Pub. Date: May 19, 2022

(54) GLYCOPHORIN A ANTIGEN-BINDING PROTEINS

(71) Applicant: ANOKION SA, Ecublens (CH)

(72) Inventors: Stephan Kontos, Boston, MA (US); Rose Jaskol Lewis, Jamaica Plain, MA (US); Julie Michelle Silverman, Cambridge, MA (US); Jian Li, Chestnut Hills, MA (US); Gregory Paul Conley, Arlington, MA (US)

(21) Appl. No.: 17/052,954

(22) PCT Filed: May 7, 2019
(86) PCT No.: PCT/IB2019/053705

§ 371 Date: Nov. 4, 2020

Related U.S. Application Data
(60) Provisional application No. 62/668,132, filed on May 7, 2018.
(60) Provisional application No. 62/805,849, filed on Feb. 14, 2019.
(60) Provisional application No. 62/829,920, filed on Apr. 5, 2019.

(51) Int. Cl. C07K 16/28

(52) U.S. Cl. CPC C07K 16/2896; C07K 2317/24; C07K 2317/92; C07K 2317/565; C07K 2317/33

(57) ABSTRACT

Several embodiments relate to antigen-binding proteins, such as antibodies, that hind to glycophorin A, In some embodiments, the antigen-binding proteins provided herein are fully human, humanized, or chimeric antibodies, binding fragments and derivatives of such antibodies, and polypeptides that specifically bind glycophorin A, Several embodiments relate to affinity-matured antigen-binding proteins. Several embodiments employ Next Generation Sequencing to predict, screen or otherwise characterize affinity matured candidates. Further still, there is disclosed nucleic acids encoding such antibodies, antibody fragments and derivatives and polypeptides, cells comprising such polynucleotides, methods of making such antibodies, antibody fragments and derivatives and polypeptides, and methods of using such antibodies, antibody fragments and derivatives and polypeptides.

### RELATED CASES

This application is the United States National Phase under 35 U.S.C. 371 of PCT Patent Application No. PCT/IB2019/053705, filed May 7, 2019, which claims the benefit of U.S. Provisional Application No. 62/668,132, filed May 7, 2018, U.S. Provisional Application No. 62/805,849, Feb. 14, 2019 and U.S. Provisional Application No. 62/829,920, filed Apr. 5, 2019, the entirety of each of which is incorporated by reference herein.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is ANOK036NP_ST25.txt, the date of creation of the ASCII text file is Apr. 26, 2021, and the size of the ASCII text file is 197 kilobytes.

### BACKGROUND

Erythrocytes, also known as red blood cells (RBCs), are the most abundant cell type in mammalian blood and express a distinctive set of cell surface markers.

### SUMMARY

The compositions and related methods summarized above and set forth in further detail below describe certain actions taken by a practitioner; however, it should be understood that they can also include the instruction of those actions by another party. Thus, actions such as “administering an anti-human glycophorin A antigen-binding construct” include “instructing the administration of an anti-human glycophorin A antigen-binding construct.”

In several embodiments, there is provided an antigen-binding protein for binding to Glycophorin A, comprising a variable light domain comprising at least a first light chain complementary determining region (LC CDR1) and a second light chain CDR (LC CDR2), a variable heavy domain comprising at least a first heavy chain complementary determining region (HC CDR1) and a second heavy chain CDR (HC CDR2), wherein the antigen-binding protein is capable of binding to at least one of human glycophorin A and cynomolgus glycophorin A, wherein the antigen-binding protein binds to human glycophorin A with a dissociation constant (Kd) of about 1 nM to about 100 nM.

In several embodiments, the antigen-binding protein is affinity matured from a parent amino acid sequence encoding an antigen-binding protein having greater Kd than the antigen-binding protein. In several embodiments, the Kd is measured by flow cytometry, surface plasmon resonance, biolayer inferometry, or radioimmunoassay.

In several embodiments, the antigen-binding protein is not a full-length antibody. In several embodiments, the antigen-binding protein does not include a constant region. In several embodiments, the antigen-binding protein has fewer variable domains than a natural antibody. In several embodiments, the antigen-binding protein is not an scFv. In several embodiments, the antigen-binding protein comprises a fragment antibody binding (Fab), wherein the Fab binds to both human glycophorin A and cynomolgus glycophorin A, and wherein the Fab binds to human glycophorin A with a dissociation constant of between about 10 μM and 0.1 nM.

In some embodiments, the antigen-binding protein comprises or consists essentially of a Fab that is capable of binding to human and/or cynomolgus Glycophorin A. In several embodiments, the antigen-binding protein has a light chain CDR2 of the sequence LNRLH (SEQ ID NO: 298). In several embodiments, the antigen-binding protein has a heavy chain CDR1 of the sequence RMTYIL (SEQ ID NO: 278). In several embodiments, the antigen-binding protein has a light chain CDR1 of the sequence FRNNK (SEQ ID NO: 287.

In several embodiments, the LC CDR2 comprises an amino acid sequence selected from the group consisting of LNRLH (SEQ ID NO: 298), LSRTS (SEQ ID NO: 295), NTRTS (SEQ ID NO: 296), NTRPS (SEQ ID NO: 297), NTRLA (SEQ ID NO: 299), NSRLS (SEQ ID NO: 300), LSRVS (SEQ ID NO: 301), LNRVS (SEQ ID NO: 302), LNRLS (SEQ ID NO: 303), NSRLH (SEQ ID NO: 304), SSRLS (SEQ ID NO: 305), SSRVS (SEQ ID NO: 306), SNRLH (SEQ ID NO: 307), NTRVS (SEQ ID NO: 308), SNRVS (SEQ ID NO: 309), HSRLS (SEQ ID NO: 310), SSRLA (SEQ ID NO: 311), FNRVN (SEQ ID NO: 312), LNRMS (SEQ ID NO: 313), LNRIS (SEQ ID NO: 314), and LSHPH (SEQ ID NO: 315). Several embodiments relate to LC CDR2 that are at least about 90%, about 95%, or about 98% homologous to the sequences listed above. In several embodiments, the CDRL2 comprises a sequence selected from any one of SEQ ID NOs: 246, 243, 244, 245, and 247-263. In several embodiments, the antigen-binding protein is matured from a parent sequence comprising a sequence of VSKLD at a corresponding location in the amino acid sequence of the variable light domain.

In several embodiments, the HC CDR1 comprises an amino acid sequence selected from the group consisting of RMTYIL (SEQ ID NO: 278), RATYIL (SEQ ID NO: 269), RNIYIL (SEQ ID NO: 270), KYTYIL (SEQ ID NO: 271), VHTYIL (SEQ ID NO: 272), RNVFIL (SEQ ID NO: 273), RNIYLL (SEQ ID NO: 274), RKTYIL (SEQ ID NO: 275), LNVYIL (SEQ ID NO: 276), KATYIL (SEQ ID NO: 277), KTVYIL (SEQ ID NO: 279), KHVYIL (SEQ ID NO: 280), RNITMIL (SEQ ID NO: 281), KDTYIL (SEQ ID NO: 282), INSYIL (SEQ ID NO: 283), QHTYIL (SEQ ID NO: 284), and RHSYIL (SEQ ID NO: 285). Several embodiments relate to HC CDR1 that are at least about 90%, about 95%, or about 98% homologous to the sequences listed above. In several embodiments, the antigen-binding protein is matured from a parent sequence comprising a sequence of KDTYML at a corresponding location in the amino acid sequence. In several embodiments, the CDR H1 comprises a sequence selected from any one of SEQ ID NOs: 227, 217-226, and 228-233.

In several embodiments, the first light chain complementary determining region 1 (CDRL1) comprises an amino acid sequence selected from FRNNK (SEQ ID NO: 287), FRNSK (SEQ ID NO: 288), FKNGK (SEQ ID NO: 289), FRNAK (SEQ ID NO: 290), FRTGK (SEQ ID NO: 291), FKNDK (SEQ ID NO: 292), and YKNGK (SEQ ID NO: 293). Several embodiments relate to LC CDR1 that are at least about 90%, about 95%, or about 98% homologous to the sequences listed above. In several embodiments, the CDRL1 comprises a sequence selected from any one of SEQ ID NOs: 235-241. In several embodiments, the antigen-binding protein is matured from a parent sequence comprising a sequence of YSNGKT at a corresponding location in the amino acid sequence.

In several embodiments, the antigen-binding protein competes for binding to glycophorin A with one or more of 10F7, Ter119, CLB-ery-1 (AME-1), EPR8200, YTH89.1, EPR8199, JC159, GYPA/280, ab40844, HI264, GPHN02, JC159, SPM599, EPR8200, GYPA/1725R, ab112201, ab114330, ab219896, BRIC 256, or fragments or derivatives thereof. In several embodiments, the antigen-binding protein targets a different epitope that the antibodies listed above, but has a higher affinity for its target than those listed.

In several embodiments, the antigen-binding protein is fused at least one immunogenic tolerogenic antigen or at least one immunogenic fragment of a tolerogenic antigen, wherein the at least one immunogenic tolerogenic antigen or at least one immunogenic fragment of a tolerogenic antigen is associated with multiple sclerosis.

In several embodiments, the antigen-binding protein is fused to at least one immunogenic fragment of a tolerogenic antigen, wherein the at least one immunogenic fragment of a tolerogenic antigen comprises an immunogenic fragment of myelin basic protein (MBP), an immunogenic fragment of myelin oligodendrocyte glycoprotein (MOG), and/or an immunogenic fragment of myelin proteolipid protein (PLP). In several embodiments, a fragment of MBP is used in conjunction with a fragment of PLP. In several embodiments, a fragment of MOG is used in conjunction with a fragment of PLP. In several embodiments, a fragment of MOG is used in conjunction with a fragment of MBP. In several embodiments, the immunogenic fragment of a tolerogenic antigen comprises at least one of SEQ ID Nos. 169-175, and 186-202. In several embodiments, the at least one immunogenic fragment of a tolerogenic antigen comprises at least one of SEQ ID Nos. 169, 171-173, 175, and 188-202. Several embodiments relate to immunogenic fragments of antigens that are at least about 90%, about 95%, or about 98% homologous to the sequences listed above. Also provided for is the use of the antigen-binding protein fused to an MS-related antigen use in the treatment of multiple sclerosis. Methods of treating MS are also provided.

In several embodiments, the antigen-binding protein is fused at least one immunogenic tolerogenic antigen or at least one immunogenic fragment of a tolerogenic antigen, wherein the at least one immunogenic tolerogenic antigen or at least one immunogenic fragment of a tolerogenic antigen is associated with Type 1 Diabetes. In several embodiments, the antigen-binding protein is fused to at least one immunogenic fragment of a tolerogenic antigen, wherein the at least one immunogenic fragment of a tolerogenic antigen comprises an immunogenic fragment of one or more of: proinsulin, insulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5. In several embodiments, the at least one immunogenic fragment of a tolerogenic antigen comprises at least one of SEQ ID Nos. 204-214. Several embodiments relate to immunogenic fragments of antigens that are at least about 90%, about 95%, or about 98% homologous to the sequences listed above. Combinations of fragments from different antigens are used as well, in certain embodiments. For example, a fragment of insulin or fragment of proinsulin can be used in conjunction with a fragment of IA-2, a fragment of GAD-65, and/or a fragment of GAD-67. Also provided for is the use of the antigen-binding protein fused to a Type I diabetes-related antigen for use in the treatment of Type I diabetes. Methods of treating Type I diabetes are also provided.

In several embodiments, the antigen-binding protein is fused at least one immunogenic tolerogenic antigen or at least one immunogenic fragment of a tolerogenic antigen, wherein the at least one immunogenic tolerogenic antigen or at least one immunogenic fragment of a tolerogenic antigen is associated with celiac disease. In several embodiments, the antigen-binding protein is fused to at least one immunogenic fragment of a tolerogenic antigen, wherein the at least one immunogenic fragment of a tolerogenic antigen comprises an immunogenic fragment of one or more of: gluten, tissue transglutaminase, high molecular weight glutenin, low molecular weight glutenin, alpha-gliadin, gamma-gliadin, omega-gliadin, hordein, secalin, avenin, and deamidated forms thereof. In several embodiments, the at least one immunogenic fragment of a tolerogenic antigen comprises at least one of SEQ ID Nos. 182-185 and 215. Several embodiments relate to immunogenic fragments of antigens that are at least about 90%, about 95%, or about 98% homologous to the sequences listed above. Combinations of antigens are also used, in several embodiments. For example, in several embodiments a fragment of gliadin is used. In additional embodiments, native gliadin and de-amidated gliadin fragments are used. In several embodiments, Also provided for is the use of the antigen-binding protein fused to a celiac disease-related antigen for use in the treatment of Celiac disease. Methods of treating celiac disease are also provided.

There are also provided methods for affinity maturing an antibody-binding protein. In several embodiments, the method comprises depleting a phage library of non-specific binders, wherein the phage library comprises a plurality of phage, each phage expressing a candidate affinity matured antibody or antibody fragment. In several embodiments, the method comprises exposing the depleted library to a target antigen and removing (e.g., through washing or other separation approach) removing phage not bound to the target antigen, and amplifying the phage that are bound to target antigen. In several embodiments, the exposing, removing, and amplifying steps are repeated a plurality of times. For example, in several embodiments, they are repeated 2, 3, 4, 5, 6, 7, 8, 9, or 10 times. In several embodiments, the repetition induces a selection pressure that results in binding of target antigen by phage expressing high affinity candidate antibodies or antibody fragments. In several embodiments, the high affinity candidate antibodies or antibody fragments are screened. In several embodiments, the screening is performed using Next Generation Sequencing. In several embodiments, the screening is performed using ELISA. In several embodiments, the optionally further comprising one or more of screening the high affinity candidate antibodies or antibody fragments using NGS and ELISA and/or evaluating the ability of the high affinity candidate antibodies or antibody fragments to be expressed in soluble form.

In several embodiments, there is provided an antigen binding protein for binding to Glycophorin A, comprising a first variable domain comprising at least a first light chain complementary determining region (LC CDR)1 and a second light chain CDR (LC CDR2), a second variable domain comprising at least a first heavy chain complementary determining region (HC CDR)1 and a second heavy chain CDR (HC CDR2), wherein the antigen binding protein is capable of binding to both human glycophorin A and cynomolgus A. In several embodiments, the antigen-binding protein binds to human glycophorin A with a dissociation constant (Kd) of about less than 20 nM as measured by flow cytometry, surface plasmon resonance, biolayer inferometry, or radioimmunoassay. In several embodiments, the antigen-binding protein is affinity matured from a parent sequence having a greater Kd than the antigen-binding protein as measured by flow cytometry, surface plasmon resonance, biolayer inferometry, or radioimmunoassay.

In several embodiments, there is provided an antigen-binding protein comprising a light chain variable domain that is at least about 95% identical to one or more of SEQ ID NOs: 243-264. In several embodiments, there is provided an antigen-binding protein comprising a light chain variable domain that is at least about 95% identical to one or more of SEQ ID NOs: 235-242. In several embodiments, there is provided an antigen-binding protein, comprising a light chain complementary determining region (CDR)1 comprising RASSNVX₁X₂MY (SEQ ID NO: 49); and a light chain CDR2 comprising X₃X₄TSX₅LAS (SEQ ID NO: 50); wherein X₁, X₂, X₃, X₄, and X₅ are each a naturally occurring amino acid; wherein X₁ is not K when X₂ is Y, X₃ is Y, X₄ is Y, and X₅ is N; wherein X₂ is not Y when X₁ is K, X₃ is Y, X₄ is Y, and X₅ is N; wherein X₃ is not Y when X₁ is K, X₂ is Y, X₄ is Y, and X₅ is N; wherein X₄ is not Y when X₁ is K, X₂ is Y, X₃ is Y, and X₅ is N; and wherein X₅ is not N when X₁ is K, X₂ is Y, X₃ is Y, and X₄ is Y.

In several embodiments, the light chain CDR1 comprises a sequence selected from the group consisting of RASSNVX₁X₂MY, wherein X₁ is selected from F, W, and Y, and wherein X₂ is selected from F, W, Y, and Q (SEQ ID NO: 51), RASSNVX₁X₂MY, wherein X₁ is selected from F, W, and Y, and wherein X₂ is selected from F, W, and Y (SEQ ID NO: 53), RASSNVX₁X₂MY, wherein X₁ is F or Y, and wherein X₂ is selected from F, W, and Y (SEQ ID NO: 55), and RASSNVX₁X₂MY, wherein X₁ is F or Y, and wherein X₂ is F, (SEQ ID NO: 57), and wherein the light chain CDR2 comprises a sequence selected from the group consisting of X₃X₄TSX₅LAS, wherein X₃ is selected from H and Y, wherein X₄ is selected from H, R, and K, and wherein X₅ is a naturally occurring amino acid (SEQ ID NO: 52), X₃X₄TSX₅LAS, wherein X₃ is H, wherein X₄ is H or R, and wherein X₅ is a naturally occurring amino acid (SEQ ID NO: 54), X₃X₄TSX₅LAS, wherein X₃ is H, wherein X₄ is H, and wherein X₅ is a naturally occurring amino acid (SEQ ID NO: 56), and X₃X₄TSX₅LAS, wherein X₃ is H, wherein X₄ is H, and wherein X₅ is V or D (SEQ ID NO: 58).

In several embodiments, the antigen-binding protein of claim 15, further comprises a light chain CDR3 comprising QQFTSSPYT (SEQ ID NO: 45).

In several embodiments, the antigen-binding protein further comprises a heavy chain CDR1 comprising GYTFNSYFMH (SEQ ID NO: 46), or a variant thereof comprising 1, 2, 3, or 4 conservative amino acid substitutions, a heavy chain CDR2 comprising GMIRPNGGTTDYNEKFKN (SEQ ID NO: 47), or a variant thereof comprising 1, 2, 3, or 4 conservative amino acid substitutions; and a heavy chain CDR3 comprising WEGSYYALDY (SEQ ID NO: 48), or a variant thereof comprising 1, 2, 3, or 4 conservative amino acid substitutions. In several embodiments, the antigen-binding protein further comprises a heavy chain variable domain that is at least about 90% identical to SEQ ID NO: 3. In several embodiments, the antigen-binding protein further comprises a heavy chain variable domain that is at least about 95% identical to SEQ ID NO: 3. In several embodiments, the antigen-binding protein further comprises a heavy chain variable domain that is at least about 99% identical to SEQ ID NO: 3.

In several embodiments, there is provided an antigen-binding protein comprising a heavy chain variable domain that is at least about 90% identical to SEQ ID NO: 3, and a light chain variable domain that is at least about 90% identical to SEQ ID NO: 1. In several embodiments, there is provided an antigen-binding protein comprising a heavy chain variable domain that is at least about 95% identical to SEQ ID NO: 3, and a light chain variable domain that is at least about 95% identical to SEQ ID NO: 1. An antigen-binding protein comprising a heavy chain variable domain that is at least about 99% identical to SEQ ID NO: 3, and a light chain variable domain that is at least about 99% identical to SEQ ID NO: 1.

In several embodiments, there is provided an antigen-binding protein comprising an amino acid sequence selected from the group consisting of SEQ ID NOS: 4-42. In several embodiments, there is provided an antigen-binding protein comprising an amino acid sequence selected from the group consisting of SEQ ID NOS: 5, 9, 11, 12, 14, 18, 19, 20, 21, 22, 24, 27, 28, 31, 38, and 42. In one embodiment there is provided an antigen-binding protein comprising the amino acid sequence of SEQ ID NO: 21. In one embodiment there is provided an antigen-binding protein comprising the amino acid sequence of SEQ ID NO: 42.

In several embodiments, there is provided a composition for inducing immune tolerance, comprising an antigen-binding protein and further comprising at least one immunogenic tolerogenic antigen or at least one immunogenic fragment of a tolerogenic antigen. In several embodiments, the composition comprises at least one immunogenic tolerogenic antigen, wherein the at least one immunogenic tolerogenic antigen comprises at least one of myelin basic protein (MBP), myelin oligodendrocyte glycoprotein (MOG), myelin proteolipid protein (PLP), a fragment or fragments of MPB, a fragment or fragments of MOG, and a fragment or fragments of PLP.

In several embodiments, the composition comprises at least one immunogenic tolerogenic antigen, wherein the at least one immunogenic tolerogenic antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5.

In several embodiments, the composition comprises at least one immunogenic tolerogenic antigen, wherein the at least one immunogenic tolerogenic antigen comprises at least one of tissue transglutaminase, high molecular weight glutenin, low molecular weight glutenin, gluten, alpha-gliadin, gamma-gliadin, omega-gliadin, hordein, secalin, avenin, and deamidated forms thereof.

In several embodiments, an antigen-binding protein is provided. Methods for treating diseases, conditions, and disorders are also provided. In some embodiments, the antigen-binding protein comprises or consists essentially of a light chain complementary determining region (CDR)1 comprising RASSNVX₁X₂MY (SEQ ID NO: 49) and a light chain CDR2 comprising X₃X₄TSX₅LAS (SEQ ID NO: 50), wherein X₁, X₂, X₃, X₄, and X₅ are each a naturally occurring amino acid. In several embodiments, X₁ is not K when X₂ is Y, X₃ is Y, X₄ is Y, and X₅ is N. In some embodiments, X₂ is not Y when X₁ is K, X₃ is Y, X₄ is Y, and X₅ is N. In some embodiments, X₃ is not Y when X₁ is K, X₂ is Y, X₄ is Y, and X₅ is N. In some embodiments, X₄ is not Y when X₁ is K, X₂ is Y, X₃ is Y, and X₅ is N. In some embodiments, X₅ is not N when X₁ is K, X₂ is Y, X₃ is Y, and X₄ is Y. In several embodiments, the light chain CDR1 comprises RASSNVX₁X₂MY, wherein X₁ is selected from F, W, and Y, and wherein X₂ is selected from F, W, Y, and Q (SEQ ID NO: 51). In some embodiments, the light chain CDR2 comprises X₃X₄TSX₅LAS, wherein X₃ is selected from H and Y, wherein X₄ is selected from H, R, and K, and wherein X₅ is a naturally occurring amino acid (SEQ ID NO: 52). In some embodiments, the light chain CDR1 comprises RASSNVX₁X₂MY, wherein X₁ is selected from F, W, and Y, and wherein X₂ is selected from F, W, and Y (SEQ ID NO: 53). In some embodiments, the light chain CDR2 comprises X₃X₄TSX₅LAS, wherein X₃ is H, wherein X₄ is H or R, and wherein X₅ is a naturally occurring amino acid (SEQ ID NO: 54). In several embodiments, the light chain CDR1 comprises RASSNVX₁X₂MY, wherein X₁ is F or Y, and wherein X₂ is selected from F, W, and Y (SEQ ID NO: 55). In some embodiments, the light chain CDR2 comprises X₃X₄TSX₅LAS, wherein X₃ is H, wherein X₄ is H, and wherein X₅ is a naturally occurring amino acid (SEQ ID NO: 56). In some embodiments, the light chain CDR1 comprises RASSNVX₁X₂MY, wherein X₁ is F or Y, and wherein X₂ is F (SEQ ID NO: 57). In some embodiments, the light chain CDR2 comprises X₃X₄TSX₅LAS, wherein X₃ is H, wherein X₄ is H, and wherein X₅ is V or D (SEQ ID NO: 58).

In some embodiments, the antigen-binding protein comprises or consists essentially of a light chain CDR1 comprising RASSNVX₁X₂MY (SEQ ID NO: 49), or a variant thereof comprising 1, 2, 3, or 4 amino acid substitutions (e.g., conservative substitutions); and a light chain CDR2 comprising X₃X₄TSX₅LAS (SEQ ID NO: 50), or a variant thereof comprising 1, 2, 3, or 4 amino acid substitutions (e.g., conservative substitutions), wherein X₁, X₂, X₃, X₄, and X₅ are each a naturally occurring amino acid. In some embodiments, the light chain CDR1 comprises RASSNVX₁X₂MY, or a variant thereof comprising 1, 2, 3, or 4 amino acid substitutions, wherein X₁ is selected from F, W, and Y, and wherein X₂ is selected from F, W, Y, and Q (SEQ ID NO: 51). In several embodiments, the light chain CDR2 comprises X₃X₄TSX₅LAS (SEQ ID NO: 52), or a variant thereof comprising 1, 2, 3, or 4 amino acid substitutions, wherein X₃ is selected from H and Y, wherein X₄ is selected from H, R, and K, and wherein X₅ is a naturally occurring amino acid (SEQ ID NO: 52). In some embodiments, the light chain CDR1 comprises RASSNVX₁X₂MY, or a variant thereof comprising 1, 2, 3, or 4 amino acid substitutions, wherein X₁ is selected from F, W, and Y, and wherein X₂ is selected from F, W, and Y (SEQ ID NO: 53). In several embodiments, the light chain CDR2 comprises X₃X₄TSX₅LAS, or a variant thereof comprising 1, 2, 3, or 4 amino acid substitutions wherein X₃ is H, wherein X₄ is H or R, and wherein X₅ is a naturally occurring amino acid (SEQ ID NO: 54). In some embodiments, the light chain CDR1 comprises RASSNVX₁X₂MY, or a variant thereof comprising 1, 2, 3, or 4 amino acid substitutions, wherein X₁ is F or Y, and wherein X₂ is selected from F, W, and Y (SEQ ID NO: 55). In several embodiments, the light chain CDR2 comprises X₃X₄TSX₅LAS, or a variant thereof comprising 1, 2, 3, or 4 amino acid substitutions, wherein X₃ is H, wherein X₄ is H, and wherein X₅ is a naturally occurring amino acid (SEQ ID NO: 56). In some embodiments, the light chain CDR1 comprises RASSNVX₁X₂MY, or a variant thereof comprising 1, 2, 3, or 4 amino acid substitutions, wherein X₁ is F or Y, and wherein X₂ is F (SEQ ID NO: 57). In several embodiments, the light chain CDR2 comprises X₃X₄TSX₅LAS, or a variant thereof comprising 1, 2, 3, or 4 amino acid substitutions, wherein X₃ is H, wherein X₄ is H, and wherein X₅ is V or D (SEQ ID NO: 58).

Also provided herein, in several embodiments, are antigen-binding proteins comprising a light chain CDR3. In some embodiment, the light chain CDR3 comprises QQFTSSPYT (SEQ ID NO: 45). In some embodiment, the light chain CDR3 comprises a variant of SEQ ID NO: 45 comprising 1, 2, 3, or 4 amino acid substitutions.

There are also provided, in several embodiments, antigen-binding proteins comprising a heavy chain CDR1, a heavy chain CDR2, and/or a heavy chain CDR3. In some embodiments, the heavy chain CDR1 comprises GYTFNSYFMH (SEQ ID NO: 46). In some embodiments, the heavy chain CDR2 comprises GMIRPNGGTTDYNEKFKN (SEQ ID NO: 47). In some embodiments, the heavy chain CDR3 comprises WEGSYYALDY (SEQ ID NO: 48). Also provided herein, in several embodiments, are variants of a heavy chain CDR1 comprising SEQ ID NO: 46, a heavy chain CDR2 comprising SEQ ID NO: 47, and/or a heavy chain CDR3 comprising SEQ ID NO: 48, wherein the variant comprises 1, 2, 3, or 4 amino acid substitutions.

In some embodiments, the antigen-binding protein comprises a heavy chain variable domain that is at least 70% (e.g., 70-75%, 75-80%, 80-85%, 85-90%, 90-95%, 95-100%, and overlapping ranges therein) identical to SEQ ID NO: 3. In some embodiments, the antigen-binding protein comprises a light chain variable domain that is at least 70% (e.g., 70-75%, 75-80%, 80-85%, 85-90%, 90-95%, 95-100%, and overlapping ranges therein) identical to SEQ ID NO: 1.

In some embodiments, the antigen-binding protein comprises the amino acid sequence of one or more of SEQ ID NOS: 4-42.

In some embodiments, the antigen-binding protein comprises an amino acid sequence at least 70% (e.g., 70-75%, 75-80%, 80-85%, 85-90%, 90-95%, 95-100%, and overlapping ranges therein) identical to SEQ ID NOS: 4-42. In several embodiments, the antigen-binding protein comprises SEQ ID NOS: 5, 9, 11, 12, 14, 18, 19, 20, 21, 22, 24, 27, 28, 31, 38, and/or 42. In one embodiment, the antigen-binding protein comprises SEQ ID NO: 21. In one embodiment, the antigen-binding protein comprises SEQ ID NO: 42.

There are also provided, in several embodiments, humanized antigen-binding proteins. In some such embodiments, the antigen-binding protein comprises one or more of the following: (a) a first human light chain framework region (FR1) selected from SEQ ID NOS: 131-140; (b) a human FR2 of the light chain framework region selected from SEQ ID NOS: 141-148; (c) a human FR3 of the light chain selected from SEQ ID NOS: 149-156; (d) a human FR4 of the light chain selected from SEQ ID NOS: 157-162; (e) a first human heavy chain framework region (FR1) selected from SEQ ID NOS: 109-115; (f) a human FR2 of the heavy chain selected from SEQ ID NOS: 116-119; (g) a human FR3 of the heavy chain selected from SEQ ID NOS: 120-127; and/or (h) a human FR4 of the heavy chain selected from SEQ ID NOS: 128-130. In some embodiments, the antigen-binding protein comprises a human constant region. In some embodiments, the human constant region may be IgG1, IgG2, IgG3 and/or IgG4. In one embodiment, the human constant region is IgG1.

In some embodiments, the antigen-binding protein is a full length antibody, while in other embodiments the antigen-binding protein is an antigen-binding fragment of an antibody. In several embodiments, the antigen-binding protein comprises a Fab, a Fab′, a F(ab′)2, a Fd, a single chain Fv or scFv, a disulfide linked Fv, a V NAR domain, a IgNar, an intrabody, an IgG-CH2, a minibody, a F(ab′)3, a tetrabody, a triabody, a diabody, a single-domain antibody, DVD-Ig, Fcab, mAb2, a (scFv)2, or a scFv-Fc.

In several embodiments, the antigen-binding protein specifically binds glycophorin A (GPA). In some embodiments, the antigen-binding protein specifically binds one or more of human GPA, cynomolgus GPA, porcine GPA, canine GPA, murine GPA, and/or rat GPA (e.g., the antigen-binding protein is bi- or multi-specific). In one embodiment, binding is specific for human GPA. In some embodiments, the antigen-binding protein binds to human GPA with a K_{d} of greater than 1.0 nM (1.0, 2.0, 3.0, 4.0, 5.0, 6.0, 7.0, 8.0, 9.0, 10.0, 12.0, 14.0, 20, 50 or higher and overlapping ranges therein). In some such embodiments, the binding affinity is measured by flow cytometry, surface plasmon resonance, biolayer inferometry, and/or radioimmunoassay.

In some embodiments, the antigen-binding protein is affinity matured. In several embodiments, the antigen-binding protein is an affinity matured variant of one or more of 10F7, Ter119, CLB-ery-1 (AME-1), EPR8200, YTH89.1, EPR8199, JC159, GYPA/280, ab40844, HI264, GPHN02, JC159, SPM599, EPR8200, GYPA/1725R, ab112201, and/or BRIC 256. In one embodiment, the antigen-binding protein is an affinity matured variant of 10F7. In several embodiments, the antigen-binding protein competes for binding with GPA with 10F7, Ter119, CLB-ery-1 (AME-1), EPR8200, YTH89.1, EPR8199, JC159, GYPA/280, ab40844, HI264, GPHN02, JC159, SPM599, EPR8200, GYPA/1725R, ab112201, BRIC 256, fragments thereof, and/or derivatives thereof. In several embodiments, the antigen-binding protein inhibits the binding of 10F7, CLB-ery-1 (AME-1), EPR8200, YTH89.1, EPR8199, JC159, GYPA/280, ab40844, HI264, GPHN02, SPM599, GYPA/1725R, ab112201, ab114330, ab219896, BRIC 256, fragments thereof, and/or derivatives thereof, to human GPA by at least 10% (e.g., 10-15%, 15-20%, 20-25%, 25-30%, 30-40%, 40-50%, 50-60%, 60-70%, 70-80%, 80-90%, 90-100%, and overlapping ranges therein). In one embodiment, the inhibition of binding is by at least about 50%.

There are provided, in several embodiments, humanized affinity-matured antigen-binding proteins specifically binding human GPA, comprising a light chain CDR1 comprising the amino acid sequence of any one of SEQ ID NO: 59, 61, 63, 65, 67, 69, 71, 73, 76, 78, 80, 81, 83, 85, 87, 88, 90, 91, 93, 95, 96, 98, 99, 101, 102, 104, 105, 107, and 108; and/or a light chain CDR2 comprising the amino acid sequence of any one of SEQ ID NO: 60, 62, 64, 66, 68, 70, 72, 74, 75, 77, 79, 82, 84, 86, 89, 92, 94, 97, 100, 103, and 106.

Also provided, in several embodiments, are antigen-binding proteins that specifically bind human GPA comprising one, two, three, four, five, six, seven, eight, nine, or ten of the following: (a) a light chain CDR1 comprising the amino acid sequence of any one of SEQ ID NO: 59, 61, 63, 65, 67, 69, 71, 73, 76, 78, 80, 81, 83, 85, 87, 88, 90, 91, 93, 95, 96, 98, 99, 101, 102, 104, 105, 107, and 108; (b) a light chain CDR2 comprising the amino acid sequence of any one of SEQ ID NO: 60, 62, 64, 66, 68, 70, 72, 74, 75, 77, 79, 82, 84, 86, 89, 92, 94, 97, 100, 103, and 106; (c) a light chain CDR3 comprising SEQ ID NO: 45, or a variant thereof comprising 1, 2, 3, or 4 conservative amino acid substitutions; (d) a heavy chain CDR1 comprising SEQ ID NO: 46, or a variant thereof comprising 1, 2, 3, or 4 conservative amino acid substitutions; (e) a heavy chain CDR2 comprising SEQ ID NO: 47, or a variant thereof comprising 1, 2, 3, or 4 conservative amino acid substitutions; (f) a heavy chain CDR3 comprising SEQ ID NO: 48, or a variant thereof comprising 1, 2, 3, or 4 conservative amino acid substitutions; (g) a first human light chain framework region (FR1) selected from SEQ ID NOS: 131-140, a human FR2 of the light chain framework region selected from SEQ ID NOS: 141-148, a human FR3 of the light chain selected from SEQ ID NOS: 149-156, and a human FR4 of the light chain selected from SEQ ID NOS: 157-162; (h) a first human heavy chain framework region (FR1) selected from SEQ ID NOS: 109-115, a human FR2 of the heavy chain selected from SEQ ID NOS: 116-119, a human FR3 of the heavy chain selected from SEQ ID NOS: 120-127, and a human FR4 of the heavy chain selected from SEQ ID NOS: 128-130; (i) a human heavy chain constant region; and/or (j) a human light chain constant region.

Also provided herein, in several embodiments, are isolated polynucleotides encoding the antigen-binding proteins described herein. In some embodiments, the isolated polynucleotide encodes a polypeptide at least 70% (e.g., 25-50%, 50-75%, 75-100%, 100-150%, or higher and overlapping ranges therein) identical to one or more of SEQ ID NOs: 1-42. Also provided herein, in several embodiments, are vectors comprising one or more of the polynucleotides described herein. Additionally, host cell lines (e.g., CHO, k1SV, XCeed, CHOK1SV, and GS-KO) comprising the vectors described herein are provided in several embodiments.

In several embodiments, methods of producing the antigen-binding proteins described herein are provided, wherein the method comprises culturing a cell line described herein under conditions wherein the antigen-binding protein is produced and recovering the antigen-binding protein. In several embodiments, the antigen-binding protein comprises one or more light chains and one or more heavy chains. In some embodiments heavy and light chains are encoded on separate vectors, while in other embodiments the heavy and light chains are encoded on the same vectors.

Pharmaceutical compositions comprising the antigen-binding protein described herein are provided in some embodiments. In some embodiments, a pharmaceutically acceptable carrier is also provided. In some embodiments, the pharmaceutical composition is formulated for intravenous administration.

In several embodiments, a composition for inducing immune tolerance is provided. In some embodiments, the composition comprises or consists essentially of an antigen-protein described herein and at least one immunogenic tolerogenic antigen or at least one immunogenic fragment of a tolerogenic antigen. In some embodiments, the tolerogenic antigen is capable of eliciting an unwanted immune response in a subject when the subject is exposed to the tolerogenic antigen. In one embodiment, the tolerogenic antigen may be an endogenous antigen (e.g., a self-antigen or self-antigens) or an exogenous antigen (e.g., a foreign antigen or antigens). In another embodiment, the tolerogenic antigen may be selected from a foreign transplant antigen against which transplant recipients develop an unwanted immune response (e.g., transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease). In several embodiments, delivery vectors (e.g., viral vectors such as adenovirus or adeno-associated virus) are used to deliver a therapeutic agent, but can themselves induce an immune response. Thus, according to several embodiments, compositions and methods are provided that induce immune tolerance to at least a portion of such vectors.

In several embodiments, methods of treatment and/or uses of the compositions described herein are provided for the prophylaxis or treatment of one or more of the following conditions: transplant rejection, Type 1 diabetes, celiac disease, and/or multiple sclerosis. In the methods of treatment, effective amounts of the composition are delivered to a subject (e.g., human or veterinary). Although unwanted immune responses are disclosed in several embodiments, other embodiments are used to treat non-immune conditions (e.g., those conditions that would benefit from targeting of a composition to an erythrocyte). Also provided, in some embodiments, are diagnostic methods and diagnostic uses of the antigen-biding proteins disclosed herein. Kits comprising one or more compounds and devices for administration (syringes, containers, inhalers, etc.), as well as instructions for use, are provided in certain embodiments.

In several embodiments, the at least one immunogenic tolerogenic antigen comprises at least one of myelin basic protein (MBP), myelin oligodendrocyte glycoprotein (MOG) myelin proteolipid protein (PLP), immunogenic fragments thereof, and immunogenic mimotopes thereof. In some embodiments, the at least one immunogenic fragment of a tolerogenic antigen comprises at least one of SEQ ID Nos. 169-175, and 186-202. In several such embodiments, uses of the composition in the treatment of multiple sclerosis are provided.

In several embodiments, the at least one immunogenic tolerogenic antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, s loop, glial fibrillary acidic protein, regenerating gene II, pancreatic duodenal homeobox 1, dystrophia myotonica kinase, SST G-protein coupled receptors 1-5, immunogenic fragments thereof, and immunogenic mimotopes thereof. In some embodiments, the at least one immunogenic fragment of a tolerogenic antigen comprises at least one of SEQ ID Nos. 204-214. In several such embodiments, uses of the composition in the treatment of Type 1 diabetes are provided.

In several embodiments, the at least one immunogenic tolerogenic antigen comprises at least one of tissue transglutaminase, high molecular weight glutenin, low molecular weight glutenin, gluten, alpha-gliadin, gamma-gliadin, omega-gliadin, hordein, secalin, avenin, deamidated forms thereof, immunogenic fragments thereof, and immunogenic mimotopes thereof. In some embodiments, the at least one immunogenic fragment of a tolerogenic antigen comprises at least one of SEQ ID Nos. 182-185 and 215. In several such embodiments, uses of the composition in the treatment of Celiac disease are provided.

In several embodiments, the at least one immunogenic tolerogenic antigen comprises at least one of a MHC class I protein, a MHC class II protein, minor blood group antigens, RhCE, Kell, Kidd, Duffy, Ss, immunogenic fragments thereof, and immunogenic mimotopes thereof. In several such embodiments, uses of the composition in the treatment or prevention of transplant rejection are provided.

According to several embodiments, there is provided an antigen-binding protein, comprising a heavy chain complementary determining region 1 (CDR H1) comprising an amino acid sequence selected from RATYIL, RNIYIL, KYTYIL, VHTYIL, RNVFIL, RNIYLL, RKTYIL, LNVYIL, KATYIL, RMTYIL, KTVYIL, KHVYIL, RNITMIL, KDTYIL, INSYIL, QHTYIL, RHSYIL (SEQ ID NOs. 269-285) and combinations thereof. In several embodiments, the antigen-binding protein is matured from a parent sequence comprising a sequence of KDTYML (SEQ ID NO: 286) at a corresponding location in the amino acid sequence.

Additional embodiments provide for an antigen-binding protein, comprising a first light chain complementary determining region 1 (CDRL1a) comprising an amino acid sequence selected from FRNNK, FRNSK, FKNGK, FRNAK, FRTGK, FKNDK, YKNGK (SEQ ID NOs: 287-293), and combinations thereof. In several embodiments, the antigen-binding protein is matured from a parent sequence comprising a sequence of YSNGKT (SEQ ID NO. 294) at a corresponding location in the amino acid sequence.

Still additional embodiments, provide for an antigen-binding protein, comprising a light chain complementary determining region (CDRL2) comprising an amino acid sequence selected from LSRTS, NTRTS, NTRPS, LNRLH, NTRLA, NSRLS, LSRVS, LNRVS, LNRLS, NSRLH, SSRLS, SSRVS, SNRLH, NTRVS, SNRVS, HSRLS, SSRLA, FNRVN, LNRMS, LNRIS, LSHPH (SEQ ID NOs: 295-315), and combinations thereof. In several embodiments, the CDLR2 is matured from a parent sequence comprising a sequence of VSKLD (SEQ ID NO. 316) at a corresponding location in the amino acid sequence.

In several embodiments, an antigen binding protein comprising a CDRH1, CDRL1a and/or CDRL2 binds a human target antigen and a cynomolgus target antigen. In several embodiments, the human target antigen and the cynomolgus target antigen are glycophorin A. In several embodiments, an antigen binding protein comprising a CDRH1, CDRL1a and/or CDRL2 is coupled to an antigen against which tolerance is desired.

In several embodiments, there is provided a composition for inducing immune tolerance, comprising an antigen-binding protein affinity matured using NGS fused (e.g., recombinantly attached, chemically conjugated, etc.) or otherwise coupled to at least one immunogenic tolerogenic antigen or at least one immunogenic fragment of a tolerogenic antigen. In several embodiments, the at least one immunogenic tolerogenic antigen comprises at least one of myelin basic protein (MBP), myelin oligodendrocyte glycoprotein (MOG), myelin proteolipid protein (PLP), a fragment or fragments of MPB, a fragment or fragments of MOG, and a fragment or fragments of PLP. In several embodiments, the at least one immunogenic fragment of a tolerogenic antigen comprises at least one of SEQ ID Nos. 169-175, and 186-202. In several embodiments, the at least one immunogenic fragment of a tolerogenic antigen comprises at least one of SEQ ID Nos. 169, 171-173, 175, and 188-202. In several embodiments, such embodiments are for use in the treatment of multiple sclerosis.

In additional embodiments, the composition comprises an antigen-binding protein affinity matured using NGS and at least one immunogenic tolerogenic antigen, wherein the at least one immunogenic tolerogenic antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2α (IA-2α), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100α, glial fibrillary acidic protein, regenerating gene II, pancreatic duodenal homeobox 1, dystrophia myotonica kinase, and SST G-protein coupled receptors 1-5. In several embodiments, the composition comprises at least one immunogenic fragment of a tolerogenic antigen, wherein the at least one immunogenic fragment of a tolerogenic antigen comprises SEQ ID Nos. 204-214. In some embodiments, such compositions are for use in the treatment of Type I diabetes.

In additional embodiments, the composition comprises an antigen-binding protein affinity matured using NGS and at least one immunogenic tolerogenic antigen, wherein the at least one immunogenic tolerogenic antigen comprises at least one of tissue transglutaminase, high molecular weight glutenin, low molecular weight glutenin, gluten, alpha-gliadin, gamma-gliadin, omega-gliadin, hordein, secalin, avenin, and deamidated forms thereof. In some embodiments, the composition comprises at least one immunogenic fragment of a tolerogenic antigen, wherein the at least one immunogenic fragment of a tolerogenic antigen comprises SEQ ID Nos. 182-185 and 215. In some embodiments, such compositions are for use in the treatment of Celiac disease.

Methods of treating multiple sclerosis, Type 1 diabetes or celiac disease are provided for herein, and comprise administering to a subject an antigen-binding protein that was affinity matured using NGS and an antigen or immunogenic fragment associated with MS, T1D or Celiac disease.

Also provided for herein is a method for affinity maturing an antibody or antibody fragment to have enhanced affinity for a target, the method comprising depleting a phage library of non-specific binders, wherein the phage library comprises a plurality of phage, each phage expressing a candidate affinity matured antibody or antibody fragment, exposing the depleted library to a target antigen, removing phage not bound to the target antigen by washing, amplifying the phage that are bound to target antigen, repeating the exposing, removing and amplifying steps a plurality of times to induce a selection pressure that results in binding of target antigen by phage expressing high affinity candidate antibodies or antibody fragments, and screening the high affinity candidate antibodies or antibody fragments using Next Generation Sequencing.

In several embodiments, the method optionally further comprising one or more of screening the high affinity candidate antibodies or antibody fragments using ELISA and evaluating the ability of the high affinity candidate antibodies or antibody fragments to be expressed in soluble form.

### BRIEF DESCRIPTION OF THE FIGURES

FIGS. 1A-1C depict the humanized 10F7 antibody sequences employed for affinity maturation campaigns. FIG. 1A depicts the sequences of the light and heavy chains of the constant and variable regions of a humanized antibody clone (“m10”) chosen as a non-limiting antibody sequence for follow-on affinity maturation. The randomized antigen-binding residues of the complementarity-determining regions in the affinity maturation campaigns are indicated by bold and underline. FIG. 1B depicts CDRs of the light and heavy chains of the variable region in accordance with the Kabat and Clothia schemes, and as well as the locations of the Paratome antigen binding regions (ABRs). FIG. 1C depicts the five separate phage display libraries that were generated by TRIM (trinucleotide mutagenesis).

FIG. 2 depicts a schematic of an affinity maturation selection process used in accordance with several embodiments. Phage display libraries were subjected to three rounds of selections against recombinant biotinylated human glycophorin A (GPA), followed by additional screening by ELISA. Positive clones were sequenced, recombinantly expressed, purified, and their affinity was characterized by ELISA, biolayer interferometry, surface plasmon resonance, and flow cytometry assays.

FIG. 3 depicts the sequences of light chain variable domains of affinity-matured fAbs. The randomized antigen-binding residues of the CDR1 and CDR2 are indicated by bold and underline.

FIG. 4 depicts sequence analysis of the 3-103 antibody Variable Light (VL) and Variable Heavy (VH) chains.

FIG. 5 depicts a non-limiting schematic of affinity maturation of antibodies (including fragments) as disclosed herein.

FIG. 6 depicts a non-limiting schematic of the process flow used to affinity mature an antibody.

FIG. 7 depicts a non-limiting embodiment of a selection scheme as disclosed herein.

FIGS. 8A-8C depict data related to the maturation campaign for the CDR-L1b library. FIG. 8A shows the convergence sequence of hits from the maturation campaign. FIG. 8B shows a schematic ribbon diagram of 3-103 with various CDR regions identified. FIG. 8C shows the degree of binding of candidate matured antibody fragments to human erythrocytes as compared to the parent antibody fragment (non-matured).

FIGS. 9A-9I depict data related to the fold sequence enrichment versus the enhanced affinity of the resultant antibodies screened from the CDRL1b library. Wild type 3-103 is indicated with an arrow for reference.

FIGS. 10A-10D depicts data related to the CDRL1a clones isolated from the culture supernatant. FIG. 10A shows the degree of binding of candidate matured antibody fragments to human erythrocytes as compared to the parent antibody fragment (non-matured). FIG. 10B shows the convergence sequence of hits from the maturation campaign. FIG. 10C shows a comparison of the number and affinities of sequences identified by ELISA and NGS. FIG. 10D shows a breakdown of the percentage of sequences that were identified by ELISA or NGS methods and can be expressed as soluble Fabs, as categorized by their affinities for human erythrocytes (note—this graph depicts the breakdown of those Fabs tested by affinity, the sum of the type of Fab across all affinity ranges equals 100%).

FIGS. 11A-11F depict data related to the fold sequence enrichment versus the enhanced affinity of the resultant antibodies screened from the CDRL1a library. Wild type 3-103 is indicated with an arrow for reference.

FIGS. 12A-12D depict data related to the CDRL2 clones isolated from the culture supernatant. FIG. 12A shows the degree of binding of candidate matured antibody fragments to human erythrocytes as compared to the parent antibody fragment (non-matured). FIG. 12B shows the convergence sequence of hits from the maturation campaign. FIG. 12C shows a comparison of the number and affinities of sequences identified by ELISA and NGS. FIG. 12D shows a breakdown of the percentage of sequences that were identified by ELISA or NGS methods and can be expressed as soluble Fabs, as categorized by their affinities for human erythrocytes (note—this graph depicts the breakdown of those Fabs tested by affinity, the sum of the type of Fab across all affinity ranges equals 100%).

FIGS. 13A-13F depict data related the fold enrichment of sequences from the CDRL2 library from the Round 1 to Round 3 screening outputs. Wild type 3-103 is indicated with an arrow for reference.

FIGS. 14A-14D depict data related to the CDRH1 clones isolated from the culture supernatant. FIG. 14A shows the degree of binding of candidate matured antibody fragments to human erythrocytes as compared to the parent antibody fragment (non-matured). FIG. 14B shows the convergence sequence of hits from the maturation campaign. FIG. 14C shows a comparison of the number and affinities of sequences identified by ELISA and NGS. FIG. 14D shows a breakdown of the percentage of sequences that were identified by ELISA or NGS methods and can be expressed as soluble Fabs, as categorized by their affinities for human erythrocytes (note—this graph depicts the breakdown of those Fabs tested by affinity, the sum of the type of Fab across all affinity ranges equals 100%).

FIGS. 15A-15F depict related the fold enrichment of sequences from the CDRH1 library from the Round 1 to Round 3 screening outputs. Wild type 3-103 is indicated with an arrow for reference.

FIGS. 16A-16D depict data related to the CDRH2 clones isolated from the culture supernatant. FIG. 16A shows the degree of binding of candidate matured antibody fragments to human erythrocytes as compared to the parent antibody fragment (non-matured). FIG. 16B shows the convergence sequence of hits from the maturation campaign. FIG. 16C shows a comparison of the number and affinities of sequences identified by ELISA, manual mutation design and NGS. FIG. 16D shows a breakdown of the percentage of sequences that were identified by ELISA, manual mutation design, or NGS methods and can be expressed as soluble Fabs, as categorized by their affinities for human erythrocytes (note—this graph depicts the breakdown of those Fabs tested by affinity, the sum of the type of Fab across all affinity ranges equals 100%).

FIGS. 17A-17I depicts depict related the fold enrichment of sequences from the CDRH2 library from the Round 1 to Round 3 screening outputs. Wild type 3-103 is indicated with an arrow for reference.

FIGS. 18A-18B depicts data related to the binding of selected clones from the CDRL1a, L2, and H1 libraries to human (18A) or cyno (18B) erythrocytes.

FIG. 19 depicts an analysis of the efficiency of enhanced affinity from the indicated CDR libraries.

FIG. 20 depicts data related to the sequences of, and human and cyno affinity, selected clones.

FIGS. 21A-21F provide summaries of the data presented in prior figures related to the comparison of enhanced antibody affinity either by ELISA-based or Next Gen Sequencing (NGS)-based techniques.

FIG. 22 depicts data that correlates an increase in affinity for a target on the red blood cell with an improvement in disease severity.

FIGS. 23A-23C depict data related to evaluation of enrichment of selected sequences that correlate to affinity improvements. FIG. 23A depicts a scatter plot of sequence fold enrichment vs. affinity from various libraries. FIG. 23B depicts affinity data from Library 1, which was enriched for wild-type and wild-type like sequences. FIG. 23C depicts affinity data from Library 4, which was enriched for non-parental sequences and showed enhanced affinity.

FIG. 24 depicts affinity data against cyno red blood cells using select clones that showed enhanced affinity for human red blood cells.

FIGS. 25A-25B depict kinetic analysis data for a clone (B) showing enhanced affinity vs. wild type (A).

FIG. 26 depicts an additional summary graph comparing ELISA and NGS screening methods.

### DETAILED DESCRIPTION

Antigen-Binding Proteins that Bind Glycophorin A

Erythrocytes, also known as red blood cells (RBCs), are the most abundant cell type in mammalian blood. They are small disc-shaped, anucleated, biconcave cells whose primary function is to carry oxygen and carbon dioxide to and from the tissues. Red blood cells express a distinctive set of cell surface markers, including the human blood group antigens, glycophorins, band 3 and the Lewis antigens.

Human Glycophorin A (GPA) is a protein which is encoded by the GYPA gene (CD235a) and refers to a single transmembrane domain protein with a heavily glycosylated extracellular domain. Glycophorin A is exclusively expressed on erythroid cells and in the blood by RBCs.

Several embodiments relate to antigen-binding proteins, such as antibodies, that bind to glycophorin A. In some embodiments, the antigen-binding proteins provided herein are fully human, humanized, or chimeric antibodies, binding fragments and derivatives of such antibodies, and polypeptides that specifically bind glycophorin A. Several embodiments relate to affinity-matured antigen-binding proteins. Further still, there is disclosed nucleic acids encoding such antibodies, antibody fragments and derivatives and polypeptides, cells comprising such polynucleotides, methods of making such antibodies, antibody fragments and derivatives and polypeptides, and methods of using such antibodies, antibody fragments and derivatives and polypeptides.

The highly homologous GPA and glycophorin B (GPB) are encoded by two genes derived one from the other after a gene duplication event. The sequence of the 26 N-terminal amino acids of GPB is identical to one of the two allelic forms of GPA N-terminus. GPA and GPB are single transmembrane domain proteins with a heavily glycosylated extracellular domain. GPA and GPB have been found to associate in the red cell membrane. In some embodiments, the antigen-binding protein binds GPA in monomeric form. In some embodiments, the antigen-binding protein binds GPA in dimeric form. In some embodiments, the antigen-binding protein binds GPB. In some embodiments, the antigen-binding protein binds GPB in monomeric form. In some embodiments, the antigen-binding protein binds GPB in dimeric form. In some embodiments, the antigen-binding protein binds a heterodimer of GPA and GPB.

Glycophorins carry several blood group antigens, for example, the M and N blood group antigens on GPA, and the N, S and s antigens on GPB. Moreover glycophorins carry antigens which are independent of blood groups and several murine monoclonal antibodies exist that target such epitopes constantly present on the molecules independently of the blood group. In some embodiments, the antigen-binding protein recognizes GPA on red cells independently of blood group antigens carried by the molecule. In some embodiments, the reactivity of the antigen-binding protein is the same regardless of whether the blood group phenotype is M+N+, M−N+, or M+N−. In some embodiments, the antigen-binding protein recognizes an epitope of GPA that is not related to the blood group determinants carried by this protein

In some embodiments, the antigen-binding protein binds GPA of a single species. In some embodiments, the antigen-binding protein binds GPA of one or more species, including, but not limited to, human, cynomolgus macaque, porcine, canine, murine and rat. In some embodiments, the antigen-binding protein binds human GPA. In some embodiments, the antigen-binding protein binds cynomolgus GPA. In some embodiments, the antigen-binding protein binds human GPA and cynomolgus GPA.

### Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is GPA or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen-binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. As used herein, the term “light chain” shall be given its ordinary meaning, and shall also refer to a polypeptide comprising, from amino terminus to carboxyl terminus, a single immunoglobulin light chain variable region (VL) and a single immunoglobulin light chain constant domain (CL). Heavy chains are classified as mu (μ), delta (Δ), gamma (γ), alpha (α), and epsilon (δ), and define the antibody's isotype as IgM, IgD, IgG, IgA, and IgE, respectively. As used herein, the term “heavy chain” shall be given its ordinary meaning, and shall also refer to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

In some embodiments, the antigen-binding protein is an antibody. As used herein, the term “antibody” shall be given its ordinary meaning, and shall also refer to an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Furthermore, unless explicitly excluded, antibodies include, for example, monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a “fragment” or “antigen-binding fragment” of an antibody. As used herein, the term “antibody fragment” shall be given its ordinary meaning, and shall also refer to the Fab, Fab′, F(ab′)2, and Fv fragments that contain at least one CDR of an immunoglobulin that is sufficient to confer specific antigen binding to GPA. Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR set forth in Table 2 herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-GPA antibody. In several embodiments, all of the CDRs are derived from an anti-GPA antibody. In some embodiments, the CDRs from more than one anti-GPA antibodies are mixed and matched in a chimeric antibody. For instance, a chimeric antibody may comprise a CDR1 from the light chain of a first anti-GPA antibody, a CDR2 and a CDR3 from the light chain of a second anti-GPA antibody, and the CDRs from the heavy chain from a third anti-GPA antibody. Further, the framework regions of antigen-binding proteins disclosed herein may be derived from one of the same anti-GPA antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity.

In some embodiments, an antigen-binding protein is provided comprising a heavy chain variable domain having at least 90% identity to any of the VH domain amino acid sequences set forth in Table 1. In some embodiments, the antigen-binding protein comprises a heavy chain variable domain having at least 95% identity to any of the VH domain amino acid sequences set forth in Table 1. In some embodiments, the antigen-binding protein comprises a heavy chain variable domain having at least 99% identity to any of the VH domain amino acid sequences set forth in Table 1. In several embodiments, the heavy chain variable domain may have one or more additional mutations in any of the VH domain amino acid sequences set forth in Table 1, but retains specific binding to GPA. In several embodiments, the heavy chain variable domain may have one or more additional mutations in any of the VH domain amino acid sequences set forth in Table 1, but has improved specific binding to GPA.

In some embodiments, the antigen-binding protein comprises a light chain variable domain having at least 90% identity to any of the VL domain amino acid sequences set forth in Table 1. In some embodiments, the antigen-binding protein comprises a light chain variable domain having at least 95% identity to any of the VL domain amino acid sequences set forth in Table 1. In some embodiments, the antigen-binding protein comprises a light chain variable domain having at least 99% identity to any of the VL domain amino acid sequences set forth in Table 1. In several embodiments, the light chain variable domain may have one or more additional mutations in any of the VL domain amino acid sequences set forth in Table 1, but retains specific binding to GPA. In several embodiments, the light chain variable domain may have one or more additional mutations in any of the VL domain amino acid sequences set forth in Table 1, but has improved specific binding to GPA.

In some embodiments, the antigen-binding protein comprises a heavy chain variable domain having at least 90% identity to any of the VH domain amino acid sequences set forth in Table 1, and a light chain variable domain having at least 90% identity to any of the VL domain amino acid sequences set forth in Table 1. In some embodiments, the antigen-binding protein comprises a heavy chain variable domain having at least 95% identity to any of the VH domain amino acid sequences set forth in Table 1, and a light chain variable domain having at least 95% identity to any of the VL domain amino acid sequences set forth in Table 1. In some embodiments, the antigen-binding protein comprises a heavy chain variable domain having at least 99% identity to any of the VH domain amino acid sequences set forth in Table 1, and a light chain variable domain having at least 99% identity to any of the VL domain amino acid sequences set forth in Table 1.

In some embodiments, the antigen-binding protein comprises a heavy chain variable domain having any of the VH domain amino acid sequences set forth in Table 1, and a light chain variable domain having any of the VL domain amino acid sequences set forth in Table 1. In some embodiments, the light-chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a light chain variable domain listed in Table 1. In some embodiments, the light-chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a heavy chain variable domain listed in Table 1.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99% or 100% identical to the polynucleotide sequence listed in Table 1. In some embodiments, the light chain variable domain comprises a sequence of amino acids that is encoded by a polynucleotide that hybridizes under moderately stringent conditions to the complement of a polynucleotide that encodes a light chain variable domain selected from the sequences listed in Table 1. In some embodiments, the light chain variable domain comprises a sequence of amino acids that is encoded by a polynucleotide that hybridizes under stringent conditions to the complement of a polynucleotide that encodes a light chain variable domain selected from the group consisting of the sequences listed in Table 1.

In some embodiments, the heavy chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a heavy chain variable domain selected from the sequences listed in Table 1. In some embodiments, the heavy chain variable domain comprises a sequence of amino acids that is encoded by a polynucleotide that hybridizes under moderately stringent conditions to the complement of a polynucleotide that encodes a heavy chain variable domain selected from the sequences listed in Table 1. In some embodiments, the heavy chain variable domain comprises a sequence of amino acids that is encoded by a polynucleotide that hybridizes under stringent conditions to the complement of a polynucleotide that encodes a heavy chain variable domain selected from the sequences listed in Table 1.

TABLE 1
SELECTED LIGHT AND HEAVY CHAIN SEQUENCES

| | | SEQ |
|---|---|---|
| Description | Amino Acid Sequence | ID NO: |
| Humanized 10F7- | EITLTQSPATLSLSPGERATLSCRASSNVKYMYWY | 1 |
| M10-Light Chain- | QQKPGQAPRLWIYYTSNLASGIPDRFSGSGSGTDY | |
| Variable Region | TLTISRLEPEDFAVYYCQQFTSSPYTFGQGTKVEVK | |
| Humanized 10F7- | RTVAAPSVFIFPPSDEQLKSGTASVVCLLNNFYPRE | 2 |
| M10-Light Chain- | AKVQWKVDNALQSGNSQESVTEQDSKDSTYSLSS | |
| Constant Region | TLTLSKADYEKHKVYACEVTHQGLSSPVTKSFNR | |
| | GEC | |
| Humanized 10F7- | EVQLLESGGGLVQPGKSLRLSCKASGYTFNSYFM | 3 |
| M10-Heavy Chain- | HWVRQAPGKGLEWVGMIRPNGGTTDYNEKFKNR | |
| Variable Region | FTLSVDKSKNTAYLQMNSLRAEDTAVYYCARWE | |
| | GSYYALDYWGQGT | |
| Humanized 10F7- | ASTKGPSVFPLAPSSKSTSGGTAALGCLVKDYFPEP | 4 |
| M10-Heavy Chain- | VTVSWNSGALTSGVHTFPAVLQSSGLYSLSSVVTV | |
| Constant Region | PSSSLGTQTYICNVNHKPSNTKVDKKVEPKSC | |
| Humanized & | EITLTQSPATLSLSPGERATLSCRASSNVFFMYWYQ | 5 |
| Affinity Matured- | QKPGQAPRLWIHHTSNLASGIPDRFSGSGSGTDYT | |
| Light Chain- | LTISRLEPEDFAVYYCQQFTSSPYTFGQGTKVEVK | |
| Variable Region | | |
| (L1 + 2-1) | | |
| Humanized & | EITLTQSPATLSLSPGERATLSCRASSNVEFMYWY | 6 |
| Affinity Matured- | QQKPGQAPRLWIFHTSELASGIPDRFSGSGSGTDYT | |
| Light Chain- | LTISRLEPEDFAVYYCQQFTSSPYTFGQGTKVEVK | |
| Variable Region | | |
| (L1 + 2-2) | | |
| Humanized & | EITLTQSPATLSLSPGERATLSCRASSNVWGMYWY | 7 |
| Affinity Matured- | QQKPGQAPRLWIHRTSVLASGIPDRFSGSGSGTDY | |
| Light Chain- | TLTISRLEPEDFAVYYCQQFTSSPYTFGQGTKVEVK | |
| Variable Region | | |
| (L1 + 2-3) | | |
| Humanized & | EITLTQSPATLSLSPGERATLSCRASSNVYDMYWY | 8 |
| Affinity Matured- | QQKPGQAPRLWIYRTSNLASGIPDRFSGSGSGTDY | |
| Light Chain- | TLTISRLEPEDFAVYYCQQFTSSPYTFGQGTKVEVK | |
| Variable Region | | |
| (L1 + 2-4) | | |
| Humanized & | EITLTQSPATLSLSPGERATLSCRASSNVYNMYWY | 9 |
| Affinity Matured- | QQKPGQAPRLWIHRTSNLASGIPDRFSGSGSGTDY | |
| Light Chain- | TLTISRLEPEDFAVYYCQQFTSSPYTFGQGTKVEVK | |
| Variable Region | | |
| (L1 + 2-5) | | |
| Humanized & | EITLTQSPATLSLSPGERATLSCRASSNVYFMYWY | 10 |
| Affinity Matured- | QQKPGQAPRLWIHHTSFLASGIPDRFSGSGSGTDY | |
| Light Chain- | TLTISRLEPEDFAVYYCQQFTSSPYTFGQGTKVEVK | |
| Variable Region | | |
| (L1 + 2-6) | | |
| Humanized & | EITLTQSPATLSLSPGERATLSCRASSNVHHMYWY | 11 |
| Affinity Matured- | QQKPGQAPRLWIHHTSLLASGIPDRFSGSGSGTDY | |
| Light Chain- | TLTISRLEPEDFAVYYCQQFTSSPYTFGQGTKVEVK | |
| Variable Region | | |
| (L1 + 2-7) | | |
| Humanized & | EITLTQSPATLSLSPGERATLSCRASSNVIDMYWYQ | 12 |
| Affinity Matured- | QKPGQAPRLWIHHTSYLASGIPDRFSGSGSGTDYT | |
| Light Chain- | LTISRLEPEDFAVYYCQQFTSSPYTFGQGTKVEVK | |
| Variable Region | | |
| (L1 + 2-8) | | |
| Humanized & | EITLTQSPATLSLSPGERATLSCRASSNVIDMYWYQ | 13 |
| Affinity Matured- | QKPGQAPRLWIHHTSYLASGIPDRFSGSGSGTDYT | |
| Light Chain- | LTISRLEPEDFAVYYCQQFTSSPYTFGQGTKVEVK | |
| Variable Region | | |
| (L1 + 2-9) | | |
| Humanized & | EITLTQSPATLSLSPGERATLSCRASSNVWFMYWY | 14 |
| Affinity Matured- | QQKPGQAPRLWIHHTSELASGIPDRFSGSGSGTDY | |
| Light Chain- | TLTISRLEPEDFAVYYCQQFTSSPYTFGQGTKVEVK | |
| Variable Region | | |
| (L1 + 2-10) | | |
| Humanized & | EITLTQSPATLSLSPGERATLSCRASSNVRKMYWY | 15 |
| Affinity Matured- | QQKPGQAPRLWIHHTSTLASGIPDRFSGSGSGTDY | |
| Light Chain- | TLTISRLEPEDFAVYYCQQFTSSPYTFGQGTKVEVK | |
| Variable Region | | |
| (L1 + 2-11) | | |
| Humanized & | EITLTQSPATLSLSPGERATLSCRASSNVAQMYWY | 16 |
| Affinity Matured- | QQKPGQAPRLWIHHTSDLASGIPDRFSGSGSGTDY | |
| Light Chain- | TLTISRLEPEDFAVYYCQQFTSSPYTFGQGTKVEVK | |
| Variable Region | | |
| (L1 + 2-12) | | |
| Humanized & | EITLTQSPATLSLSPGERATLSCRASSNVVHMYWY | 17 |
| Affinity Matured- | QQKPGQAPRLWIAHTSELASGIPDRFSGSGSGTDY | |
| Light Chain- | TLTISRLEPEDFAVYYCQQFTSSPYTFGQGTKVEVK | |
| Variable Region | | |
| (L1 + 2-13) | | |
| Humanized & | EITLTQSPATLSLSPGERATLSCRASSNVHQMYWY | 18 |
| Affinity Matured- | QQKPGQAPRLWIHHTSKLASGIPDRFSGSGSGTDY | |
| Light Chain- | TLTISRLEPEDFAVYYCQQFTSSPYTFGQGTKVEVK | |
| Variable Region | | |
| (L1 + 2-14) | | |
| Humanized & | EITLTQSPATLSLSPGERATLSCRASSNVFYMYWY | 19 |
| Affinity Matured- | QQKPGQAPRLWIHHTSWLASGIPDRFSGSGSGTDY | |
| Light Chain- | TLTISRLEPEDFAVYYCQQFTSSPYTFGQGTKVEVK | |
| Variable Region | | |
| (L1 + 2-15) | | |
| Humanized & | EITLTQSPATLSLSPGERATLSCRASSNVFQMYWY | 20 |
| Affinity Matured- | QQKPGQAPRLWIHHTSELASGIPDRFSGSGSGTDY | |
| Light Chain- | TLTISRLEPEDFAVYYCQQFTSSPYTFGQGTKVEVK | |
| Variable Region | | |
| (L1 + 2-16) | | |
| Humanized & | EITLTQSPATLSLSPGERATLSCRASSNVYFMYWY | 21 |
| Affinity Matured- | QQKPGQAPRLWIHHTSDLASGIPDRFSGSGSGTDY | |
| Light Chain- | TLTISRLEPEDFAVYYCQQFTSSPYTFGQGTKVEVK | |
| Variable Region | | |
| (L1 + 2-17) | | |
| Humanized & | EITLTQSPATLSLSPGERATLSCRASSNVYQMYWY | 22 |
| Affinity Matured- | QQKPGQAPRLWIHHTSFLASGIPDRFSGSGSGTDY | |
| Light Chain- | TLTISRLEPEDFAVYYCQQFTSSPYTFGQGTKVEVK | |
| Variable Region | | |
| (L1 + 2-18) | | |
| Humanized & | EITLTQSPATLSLSPGERATLSCRASSNVYFMYWY | 23 |
| Affinity Matured- | QQKPGQAPRLWIHHTSELASGIPDRFSGSGSGTDY | |
| Light Chain- | TLTISRLEPEDFAVYYCQQFTSSPYTFGQGTKVEVK | |
| Variable Region | | |
| (L1 + 2-20) | | |
| Humanized & | EITLTQSPATLSLSPGERATLSCRASSNVYFMYWY | 24 |
| Affinity Matured- | QQKPGQAPRLWIHHTSVLASGIPDRFSGSGSGTDY | |
| Light Chain- | TLTISRLEPEDFAVYYCQQFTSSPYTFGQGTKVEVK | |
| Variable Region | | |
| (L1 + 2-21) | | |
| Humanized & | EITLTQSPATLSLSPGERATLSCRASSNVHSMYWY | 25 |
| Affinity Matured- | QQKPGQAPRLWIHHTSYLASGIPDRFSGSGSGTDY | |
| Light Chain- | TLTISRLEPEDFAVYYCQQFTSSPYTFGQGTKVEVK | |
| Variable Region | | |
| (L1 + 2-22) | | |
| Humanized & | EITLTQSPATLSLSPGERATLSCRASSNVWGMYWY | 26 |
| Affinity Matured- | QQKPGQAPRLWIHHTSELASGIPDRFSGSGSGTDY | |
| Light Chain- | TLTISRLEPEDFAVYYCQQFTSSPYTFGQGTKVEVK | |
| Variable Region | | |
| (L1 + 2-23) | | |
| Humanized & | EITLTQSPATLSLSPGERATLSCRASSNVQQMYWY | 27 |
| Affinity Matured- | QQKPGQAPRLWIHHTSILASGIPDRFSGSGSGTDYT | |
| Light Chain- | LTISRLEPEDFAVYYCQQFTSSPYTFGQGTKVEVK | |
| Variable Region | | |
| (L1 + 2-24) | | |
| Humanized & | EITLTQSPATLSLSPGERATLSCRASSNVAWMYWY | 28 |
| Affinity Matured- | QQKPGQAPRLWIYHTSKLASGIPDRFSGSGSGTDY | |
| Light Chain- | TLTISRLEPEDFAVYYCQQFTSSPYTFGQGTKVEVK | |
| Variable Region | | |
| (L1 + 2-8-25) | | |
| Humanized & | EITLTQSPATLSLSPGERATLSCRASSNVSFMYWYQ | 29 |
| Affinity Matured- | QKPGQAPRLWIHHTSKLASGIPDRFSGSGSGTDYT | |
| Light Chain- | LTISRLEPEDFAVYYCQQFTSSPYTFGQGTKVEVK | |
| Variable Region | | |
| (L1 + 2-26) | | |
| Humanized & | EITLTQSPATLSLSPGERATLSCRASSNVYEMYWY | 30 |
| Affinity Matured- | QQKPGQAPRLWIHHTSQLASGIPDRFSGSGSGTDY | |
| Light Chain- | TLTISRLEPEDFAVYYCQQFTSSPYTFGQGTKVEVK | |
| Variable Region | | |
| (L1 + 2-27) | | |
| Humanized & | EITLTQSPATLSLSPGERATLSCRASSNVYWMYWY | 31 |
| Affinity Matured- | QQKPGQAPRLWIHHTSDLASGIPDRFSGSGSGTDY | |
| Light Chain- | TLTISRLEPEDFAVYYCQQFTSSPYTFGQGTKVEVK | |
| Variable Region | | |
| (L1 + 2-28) | | |
| Humanized & | EITLTQSPATLSLSPGERATLSCRASSNVSQMYWY | 32 |
| Affinity Matured- | QQKPGQAPRLWIHHTSGLASGIPDRFSGSGSGTDY | |
| Light Chain- | TLTISRLEPEDFAVYYCQQFTSSPYTFGQGTKVEVK | |
| Variable Region | | |
| (L1 + 2-29) | | |
| Humanized & | EITLTQSPATLSLSPGERATLSCRASSNVTDMYWY | 33 |
| Affinity Matured- | QQKPGQAPRLWIHHTSWLASGIPDRFSGSGSGTDY | |
| Light Chain- | TLTISRLEPEDFAVYYCQQFTSSPYTFGQGTKVEVK | |
| Variable Region | | |
| (L1 + 2-30) | | |
| Humanized & | EITLTQSPATLSLSPGERATLSCRASSNVDWMYWY | 34 |
| Affinity Matured- | QQKPGQAPRLWIHHTSFLASGIPDRFSGSGSGTDY | |
| Light Chain- | TLTISRLEPEDFAVYYCQQFTSSPYTFGQGTKVEVK | |
| Variable Region | | |
| (L1 + 2-31) | | |
| Humanized & | EITLTQSPATLSLSPGERATLSCRASSNVRKMYWY | 35 |
| Affinity Matured- | QQKPGQAPRLWIHSTSGLASGIPDRFSGSGSGTDY | |
| Light Chain- | TLTISRLEPEDFAVYYCQQFTSSPYTFGQGTKVEVK | |
| Variable Region | | |
| (L1 + 2-32) | | |
| Humanized & | EITLTQSPATLSLSPGERATLSCRASSNVASMYWY | 36 |
| Affinity Matured- | QQKPGQAPRLWIHRTSVLASGIPDRFSGSGSGTDY | |
| Light Chain- | TLTISRLEPEDFAVYYCQQFTSSPYTFGQGTKVEVK | |
| Variable Region | | |
| (L1 + 2-33) | | |
| Humanized & | EITLTQSPATLSLSPGERATLSCRASSNVHGMYWY | 37 |
| Affinity Matured- | QQKPGQAPRLWIHHTSALASGIPDRFSGSGSGTDY | |
| Light Chain- | TLTISRLEPEDFAVYYCQQFTSSPYTFGQGTKVEVK | |
| Variable Region | | |
| (L1 + 2-34) | | |
| Humanized & | EITLTQSPATLSLSPGERATLSCRASSNVYWMYWY | 38 |
| Affinity Matured- | QQKPGQAPRLWIHHTSELASGIPDRFSGSGSGTDY | |
| Light Chain- | TLTISRLEPEDFAVYYCQQFTSSPYTFGQGTKVEVK | |
| Variable Region | | |
| (L1 + 2-35) | | |
| Humanized & | EITLTQSPATLSLSPGERATLSCRASSNVHHMYWY | 39 |
| Affinity Matured- | QQKPGQAPRLWIHHTSKLASGIPDRFSGSGSGTDY | |
| Light Chain- | TLTISRLEPEDFAVYYCQQFTSSPYTFGQGTKVEVK | |
| Variable Region | | |
| (L1 + 2-36) | | |
| Humanized & | EITLTQSPATLSLSPGERATLSCRASSNVRGMYWY | 40 |
| Affinity Matured- | QQKPGQAPRLWIHHTSNLASGIPDRFSGSGSGTDY | |
| Light Chain- | TLTISRLEPEDFAVYYCQQFTSSPYTFGQGTKVEVK | |
| Variable Region | | |
| (L1 + 2-37) | | |
| Humanized & | EITLTQSPATLSLSPGERATLSCRASSNVHEMYWY | 41 |
| Affinity Matured- | QQKPGQAPRLWIHHTSYLASGIPDRFSGSGSGTDY | |
| Light Chain- | TLTISRLEPEDFAVYYCQQFTSSPYTFGQGTKVEVK | |
| Variable Region | | |
| (L1 + 2-38) | | |
| Humanized & | EITLTQSPATLSLSPGERATLSCRASSNVFFMYWYQ | 42 |
| Affinity Matured- | QKPGQAPRLWIHHTSVLASGIPDRFSGSGSGTDYT | |
| Light Chain- | LTISRLEPEDFAVYYCQQFTSSPYTFGQGTKVEVK | |
| Variable Region | | |
| (L1 + 2-39) | | |

In some embodiments, there are provided antigen-binding proteins comprising one or more VH CDR1, VH CDR2 and/or VH CDR3 domains having an amino acid sequence identical to or comprising 1, 2, or 3 amino acid residue substitutions, deletions and/or insertions in each CDR relative to the VH CDR1, VH CDR2 or VH CDR3 sequences set forth in Table 2. In some embodiments, the antigen-binding protein comprises one or more VL CDR1, VL CDR2 and/or VL CDR3 having an amino acid sequence identical to or comprising 1, 2, or 3 amino acid residue substitutions, deletions and/or insertions in each CDR relative to the VL CDR1, VL CDR2 or VL CDR3 sequences set forth in Table 2. In some embodiments, the antigen-binding protein comprises one or more VH CDR1, VH CDR2 and/or VH CDR3 having an amino acid sequence identical to or comprising 1, 2, or 3 amino acid residue substitutions, deletions or insertions in each CDR relative to the VH CDR1, VH CDR2 or VH CDR3 sequences set forth in Table 2, and one or more VL CDR1, VL CDR2 and/or VL CDR3 having an amino acid sequence identical to or comprising 1, 2, or 3 amino acid residue substitutions, deletions and/or insertions in each CDR relative to the VL CDR1, VL CDR2 or VL CDR3 sequences set forth in Table 2. In some embodiments, the antigen-binding protein comprises one VH CDR1, VH CDR2 and/or VH CDR3 having an amino acid sequence identical to or comprising 1, 2, or 3 amino acid residue substitutions, deletions and/or insertions in each CDR relative to the VH CDR1, VH CDR2 or VH CDR3 sequences set forth in Table 2, and one VL CDR1, VL CDR2 and/or VL CDR3 having an amino acid sequence identical to or comprising 1, 2, or 3 amino acid residue substitutions, deletions and/or insertions in each CDR relative to the VL CDR1, VL CDR2 or VL CDR3 sequences set forth in Table 2. In some embodiments, the antigen-binding protein comprises two VH CDR1, VH CDR2 and/or VH CDR3 having an amino acid sequence identical to or comprising 1, 2, or 3 amino acid residue substitutions, deletions and/or insertions in each CDR relative to the VH CDR1, VH CDR2 or VH CDR3 sequences set forth in Table 2, and two VL CDR1, VL CDR2 and/or VL CDR3 having an amino acid sequence identical to or comprising 1, 2, or 3 amino acid residue substitutions, deletions and/or insertions in each CDR relative to the VL CDR1, VL CDR2 or VL CDR3 sequences set forth in Table 2. In some embodiments, the antigen-binding protein comprises the VH CDR1, VH CDR2 and VH CDR3 having an amino acid sequence identical to or comprising 1, 2, or 3 amino acid residue substitutions, deletions and/or insertions in each CDR relative to the VH CDR1, VH CDR2 and VH CDR3 sequences set forth in Table 2, and the VL CDR1, VL CDR2 and VL CDR3 having an amino acid sequence identical to or comprising 1, 2, or 3 amino acid residue substitutions, deletions and/or insertions in each CDR relative to the VL CDR1, VL CDR2 and VL CDR3 sequences set forth in Table 2. In some embodiments, the antigen-binding protein comprises the VH CDR1, VH CDR2, VH CDR3, VL CDR1, VL CDR2, and VL CDR3 having an amino acid sequence identical to any of the VH CDR1, VH CDR2, VH CDR3, VL CDR1, VL CDR2, and VL CDR3 sequences set forth in Table 2. In several embodiments, the VL CDR1, VL CDR2 and/or VL CDR3 may have one or more additional mutations in any of the VL CDR1, VL CDR2 and/or VL CDR3 sequences set forth in Table 2, but retains specific binding to GPA (e.g., human GPA and/or murine and/or cynomolgus macaque, depending on the embodiment). In several embodiments, the VL CDR1, VL CDR2 and/or VL CDR3 may have one or more additional mutations in any of the VL CDR1, VL CDR2 and/or VL CDR3 amino acid sequences set forth in Table 2, but has improved specific binding to GPA (e.g., human GPA and/or murine and/or cynomolgus macaque, depending on the embodiment). In several embodiments, the VH CDR1, VH CDR2 and/or VH CDR3 may have one or more additional mutations in any of the VH CDR1, VH CDR2 and/or VH CDR3 sequences set forth in Table 2, but retains specific binding to GPA (e.g., human GPA and/or murine and/or cynomolgus macaque, depending on the embodiment). In several embodiments, the VH CDR1, VH CDR2 and/or VH CDR3 may have one or more additional mutations in any of the VH CDR1, VH CDR2 and/or VH CDR3 amino acid sequences set forth in Table 2, but has improved specific binding to GPA (e.g., human GPA and/or murine and/or cynomolgus macaque, depending on the embodiment).

TABLE 2
SELECTED CDR SEQUENCES

| | | SEQ |
|---|---|---|
| | | ID |
| Description | Amino Acid Sequence | NO: |
| CDR-L1 | RASSNVKYMY | 43 |
| CDR-L2 | YYTSNLAS | 44 |
| CDR-L3 | QQFTSSPYT | 45 |
| CDR-H1 | GYTFNSYFMH | 46 |
| CDR-H2 | GMIRPNGGTTDYNEKFKN | 47 |
| CDR-H3 | WEGSYYALDY | 48 |
| CDR-L1 | RASSNVX₁X₂MY | 49 |
| | X₁, X₂, are each a naturally occurring amino acid | |
| CDR-L2 | X₃X₄TSX₅LAS | 50 |
| | X₃, X₄, and X₅ are each a naturally occurring amino acid | |
| CDR-L1 | RASSNVX₁X₂MY | 51 |
| | X₁ is selected from F, W, and Y; wherein X₂ is selected from F, | |
| | W, Y, and Q | |
| CDR-L2 | light chain CDR2 comprises X₃X₄TSX₅LAS | 52 |
| | wherein X₃ is selected from H and Y; wherein X₄ is selected | |
| | from H, R, and K; and wherein X₅ is a naturally occurring amino | |
| | acid | |
| CDR-L1 | wherein the light chain CDR1 comprises RASSNVX₁X₂MY | 53 |
| | wherein X₁ is selected from F, W, and Y; wherein X₂ is selected | |
| | from F, W, and Y | |
| CDR-L2 | light chain CDR2 comprises X₃X₄TSX₅LAS | 54 |
| | wherein X₃ is H; wherein X₄ is H or R; and wherein X₅ is a | |
| | naturally occurring amino acid | |
| CDR-L1 | wherein the light chain CDR1 comprises RASSNVX₁X₂MY | 55 |
| | wherein X₁ is F or Y; wherein X₂ is selected from F, W, and Y; | |
| CDR-L2 | the light chain CDR2 comprises X₃X₄TSX₅LAS | 56 |
| | wherein X₃ is H; wherein X₄ is H; and wherein X₅ is a naturally | |
| | occurring amino acid | |
| CDR-L1 | wherein the light chain CDR1 comprises RASSNVX₁X₂MY | 57 |
| | wherein X₁ is F or Y; wherein X₂ is F | |
| CDR-L2 | the light chain CDR2 comprises X₃X₄TSX₅LAS | 58 |
| | wherein X₃ is H; wherein X₄ is H; and wherein X₅ is V or D | |
| CDR-L1 | RASSNVFFMY | 59 |
| CDR-L2 | HHTSNLAS | 60 |
| CDR-L1 | RASSNVEFMY | 61 |
| CDR-L2 | FHTSELAS | 62 |
| CDR-L1 | RASSNVWGMY | 63 |
| CDR-L2 | HRTSVLAS | 64 |
| CDR-L1 | RASSNVYDMYW | 65 |
| CDR-L2 | YRTSNLAS | 66 |
| CDR-L1 | RASSNVYNMY | 67 |
| CDR-L2 | HRTSNLAS | 68 |
| CDR-L1 | RASSNVYFMY | 69 |
| CDR-L2 | HHTSFLAS | 70 |
| CDR-L1 | RASSNVHHMY | 71 |
| CDR-L2 | HHTSLLAS | 72 |
| CDR-L1 | RASSNVIDMY | 73 |
| CDR-L2 | HHTSYLAS | 74 |
| CDR-L2 | HHTSDLAS | 75 |
| CDR-L1 | RASSNVWFMY | 76 |
| CDR-L2 | HHTSELAS | 77 |
| CDR-L1 | RASSNVRKMY | 78 |
| CDR-L2 | HHTSTLAS | 79 |
| CDR-L1 | RASSNVAQMY | 80 |
| CDR-L1 | RASSNVVHMY | 81 |
| CDR-L2 | AHTSELAS | 82 |
| CDR-L1 | RASSNVHQMY | 83 |
| CDR-L2 | HHTSKLAS | 84 |
| CDR-L1 | RASSNVFYMY | 85 |
| CDR-L2 | HHTSWLAS | 86 |
| CDR-L1 | RASSNVFQMY | 87 |
| CDR-L1 | RASSNVYQMY | 88 |
| CDR-L2 | HHTSVLAS | 89 |
| CDR-L1 | RASSNVHSMY | 90 |
| CDR-L1 | RASSNVQQMY | 91 |
| CDR-L2 | HHTSILAS | 92 |
| CDR-L1 | RASSNVAWM | 93 |
| CDR-L2 | YHTSKLAS | 94 |
| CDR-L1 | RASSNVSFMY | 95 |
| CDR-L1 | RASSNVYEMY | 96 |
| CDR-L2 | HHTSQLAS | 97 |
| CDR-L1 | RASSNVYWMY | 98 |
| CDR-L1 | RASSNVSQM | 99 |
| CDR-L2 | HHTSGLAS | 100 |
| CDR-L1 | RASSNVTDMY | 101 |
| CDR-L1 | RASSNVDWMY | 102 |
| CDR-L2 | HSTSGLAS | 103 |
| CDR-L1 | RASSNVASMY | 104 |
| CDR-L1 | RASSNVHGMY | 105 |
| CDR-L2 | HHTSALAS | 106 |
| CDR-L1 | RASSNVRGMY | 107 |
| CDR-L1 | RASSNVHEMY | 108 |

### Humanization of Antigen-Binding Proteins

There are provided, in several embodiments, humanized antigen-binding proteins. As used herein, the term “humanized antigen-binding protein” shall be given its ordinary meaning, and shall also refer to an antigen-binding protein has a sequence that differs from the sequence of antigen-binding protein derived from a non-human species by one or more amino acid substitutions, deletions, and/or additions, such that the humanized antigen-binding protein is less likely to induce an immune response, and/or induces a less severe immune response, as compared to the non-human species antigen-binding protein, when it is administered to a human subject. In some embodiments, certain amino acids in the framework and/or constant domains of the heavy and/or light chains of the non-human species antigen-binding protein are mutated to produce the humanized antigen-binding protein. In another embodiment, the constant domain(s) from a human antigen-binding protein are fused to the variable domain(s) of a non-human species. In another embodiment, one or more amino acid residues in one or more CDR sequences of a non-human antigen-binding protein are changed to reduce the likely immunogenicity of the non-human antigen-binding protein when it is administered to a human subject, wherein the changed amino acid residues either are not residues that are primarily responsible for immunospecific binding of the antigen-binding protein to its antigen, or the changes to the amino acid sequence that are made are conservative changes, such that the binding of the humanized antigen-binding protein to the antigen is not significantly changed as compared to the binding of the non-human antigen-binding protein to the antigen.

According to several embodiments, humanization of the antigen-binding protein comprises CDR grafting that involves recombining the CDRs of a non-human antigen-binding protein and in some embodiments, only these CDRs) onto a human variable region framework and a human constant region. In several embodiments, this change substantially reduces, or even eliminates, immunogenicity. In several embodiments, allotypic or idiotypic differences may still exist, though overall immunogenicity is reduced. Some framework residues of the original antigen-binding protein may need to be preserved, depending on the embodiment. Such framework residues are amenable to identification through computer modeling or may potentially be identified by comparing known antigen-binding site structures. The residues that potentially affect antigen binding fall into several groups. The first group comprises residues that are contiguous with the antigen site surface, which could therefore make direct contact with antigens. These residues include, in several embodiments, the amino-terminal residues and those adjacent to the CDRs. The second group includes, in several embodiments, residues that could alter the structure or relative alignment of the CDRs, either by contacting the CDRs or another peptide chain in the antigen-binding protein. The third group, according to several embodiments, comprises amino acids with buried side chains that could influence the structural integrity of the variable domains.

In some embodiments the antigen-binding protein further comprises one or more of the non-limiting embodiments of FRs (framework regions) illustrated herein in Tables 3-4. In some embodiments, the antigen-binding protein comprises one or more of a heavy chain FR1 sequence, a heavy chain FR2 sequence, a heavy chain FR3 sequence, and a heavy chain FR4 sequence illustrated in Table 3. In some embodiments, the antigen-binding protein comprises one or more of a light chain FR1 sequence, a light chain FR2 sequence, a light chain FR3 sequence, and a light chain FR4 sequence illustrated in Table 4. In some embodiments, any one or more of the FR regions in Tables 3 and 4 can be combined with any one or more of the CDR sequences provided herein. Thus, in some embodiments, there are provided antigen-binding proteins that include 6 CDRs (e.g., HCDR1, HCDR2, HCDR3, LCDR1, LCDR2, and LCDR3) and 8 FRs (e.g. HFR1, HFR2, HFR3, HFR4, LFR1, LFR2, LFR3, and LFR4).

TABLE 3
SELECTED HUMANIZED HEAVY CHAIN FRAMEWORK REGIONS

| Heavy | | |
|---|---|---|
| Chain | | |
| Framework | | SEQ |
| Region | Amino Acid Sequence | ID NO: |
| FR1 | | |
| | EVQLLESGGGLVQPGKSLRLSCKAS | 109 |
| | QVQLVQSGAEVKKPGASVKVSCKAS | 110 |
| | QVQLVQSGAEVKKPGSSVKVSCKAS | 111 |
| | EVQLVQSGAEVKKPGATVKISCKVS | 112 |
| | EVQLVQSGAEVKKPGESLKISCKGS | 113 |
| | EVQLVQSGAEVKKPGESLRISCKGS | 114 |
| | QVQLVQSGSELKKPGASVKVSCKAS | 115 |
| FR2 | | |
| | WVRQAPGKGLEWV | 116 |
| | WVRQAPGQGLEWM | 117 |
| | WVQQAPGKGLEWM | 118 |
| | WVRQMPGKGLEWM | 119 |
| FR3 | | |
| | RFTLSVDKSKNTAYLQMNSLRAEDTAVYYCAR | 120 |
| | RVTMTTDTSTSTAYMELRSLRSDDTAVYYCAR | 121 |
| | RVTITADESTSTAYMELSSLRSEDTAVYYCAR | 122 |
| | RVTITADKSTSTAYMELSSLRSEDTAVYYCAR | 123 |
| | RVTITADTSTDTAYMELSSLRSEDTAVYYCAT | 124 |
| | QVTISADKSISTAYLQWSSLKASDTAMYYCAR | 125 |
| | HVTISADKSISTAYLQWSSLKASDTAMYYCAR | 126 |
| | RFVFSLDTSVSTAYLQISSLKAEDTAVYYCAR | 127 |
| FR4 | | |
| | WGQGT | 128 |
| | WGRGT | 129 |
| | WGKGT | 130 |

TABLE 4
HUMANIZED LIGHT CHAIN FRAMEWORK REGIONS

| Light | | |
|---|---|---|
| Chain | | |
| Framework | | SEQ |
| Region | Amino Acid Sequence | ID NO: |
| FR1 | | |
| | EITLTQSPATLSLSPGERATLSC | 131 |
| | EIVMTQSPATLSVSPGERATLSC | 132 |
| | EIVLTQSPATLSLSPGERATLSC | 133 |
| | DIQMTQSPSSLSASVGDRVTITC | 134 |
| | NIQMTQSPSAMSASVGDRVTITC | 135 |
| | AIQLTQSPSSLSASVGDRVTITC | 136 |
| | DIQLTQSPSFLSASVGDRVTITC | 137 |
| | AIRMTQSPFSLSASVGDRVTITC | 138 |
| | AIQMTQSPSSLSASVGDRVTITC | 139 |
| | DIQMTQSPSTLSASVGDRVTITC | 140 |
| FR2 | | |
| | WYQQKPGQAPRLWI | 141 |
| | WYQQKPGQAPRLLI | 142 |
| | WYQQKPGKAPKLLI | 143 |
| | WYQQKPGKVPKLLI | 144 |
| | WYQQKPGKAPKRLI | 145 |
| | WFQQKPGKVPKHLI | 146 |
| | WFQQKPGKAPKSLI | 147 |
| | WYQQKPAKAPKLFI | 148 |
| FR3 | | |
| | GIPDRFSGSGSGTDYTLTISRLEPEDFAVY | 149 |
| | YC | |
| | GIPARFSGSGSGTEFTLTISSLQSEDFAVYY | 150 |
| | C | |
| | GIPARFSGSGSGTDFTLTISSLEPEDFAVYY | 151 |
| | C | |
| | GVPSRFSGSGSGTDFTLTISSLQPEDFATYY | 152 |
| | C | |
| | GVPSRFSGSGSGTDFTLTISSLQPEDVATY | 153 |
| | YC | |
| | GVPSRFSGSGSGTEFTLTISSLQPEDFATYY | 154 |
| | C | |
| | GVPSRFSGSGSGTDYTLTISSLQPEDFATY | 155 |
| | YC | |
| | GVPSRFSGSGSGTEFTLTISSLQPDDFATYY | 156 |
| | C | |
| FR4 | | |
| | FGQGTKVEVK | 157 |
| | FGQGTKVEIK | 158 |
| | FGQGTKLEIK | 159 |
| | FGPGTKVDIK | 160 |
| | FGGGTKVEIK | 161 |
| | FGQGTRLEIK | 162 |

### Affinity Maturation of Antigen-Binding Proteins

There is provided, in some embodiments, antigen-binding proteins with increased affinity to a target antigen. In some embodiments, the target antigen is glycophorin A. In some embodiments, affinity maturation of the one or more of the CDRs depicted in Table 1 is obtained by a number of affinity maturation protocols including, but not limited to, maintaining the CDRs, chain shuffling, use of mutation strains of E. coli, DNA shuffling, phage display and additional PCR techniques.

Affinity can be determined using a variety of techniques, for example but not limited to, equilibrium methods (e.g., enzyme-linked immunoabsorbent assay (ELISA); or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. One example of a competitive binding assay is a radioimmunoas say comprising the incubation of labeled antigen with the antigen-binding protein in the presence of increasing amounts of unlabeled antigen, and the detection of the antigen-binding protein bound to the labeled antigen. The affinity of the antigen-binding protein for a particular antigen and the binding off-rates can be determined from the data by Scatchard plot analysis. Competition with a second antigen-binding protein can also be determined using radioimmunoassays. In this case, the antigen is incubated with antigen-binding protein conjugated to a labeled compound in the presence of increasing amounts of an unlabeled second antigen-binding protein.

An antigen-binding protein “specifically binds” to an antigen, such as GPA, if it binds to the antigen with a tight binding affinity as determined by a equilibrium dissociation constant (K_{D}, or corresponding K_{D}, as defined below) value of 10⁻⁷ M or less. An antigen-binding protein that specifically binds to human GPA may be able to bind to GPA from other species as well with the same or different affinities.

In some embodiments, there is provided antigen-binding proteins (e.g., an antibody or fragment) that specifically bind GPA with an equilibrium dissociation constant or K_{D} (k_{off}/kₒₙ) of less than 10⁻⁷ M, or of less than 10⁻⁸ M, or of less than 10⁻⁹ M, or of less than 10⁻¹⁰ M, or of less than 10⁻¹¹ M, or of less than 10⁻¹² M, or of less than 10⁻¹³ M, or of less than 5×10⁻¹³ M (lower values indicating greater binding affinity). In some embodiments, there are provided antigen-binding proteins that specifically bind GPA with an equilibrium dissociation constant or K_{D} (k_{off}/kₒₙ) of less than about 10⁻⁷ M, or of less than about 10⁻⁸ M, or of less than about 10⁻⁹ M, or of less than about 10⁻¹⁰ M, or of less than about 10⁻¹¹ M, or of less than about 10⁻¹² M, or of less than about 10⁻¹³ M, or of less than about 5×10⁻¹³ M.

In some embodiments, there is provided antigen-binding proteins (e.g., an antibody fragment) that specifically bind GPA has an equilibrium dissociation constant or K_{D} (k_{off}/kₒₙ) of between about 10⁻⁷ M and about 10⁻⁸ M, between about 10⁻⁸ M and about 10⁻⁹ M, between about 10⁻⁹ M and about 10⁻¹⁰ M, between about 10⁻¹⁰ M and about 10⁻¹¹ M, between about 10⁻¹¹ M and about 10⁻¹² M, between about 10⁻¹² M and about 10⁻¹³ M. In some embodiments, there is provided antigen-binding proteins that specifically bind GPA has an equilibrium dissociation constant or K_{D} (k_{off}/kₒₙ) of between 10⁻⁷ M and 10⁻⁸ M, between 10⁻⁸ M and 10⁻⁹ M, between 10⁻⁹ M and 10⁻¹⁰ M, between 10⁻¹⁰ M and 10⁻¹¹ M, between 10⁻¹¹ M and 10⁻¹² M, between 10⁻¹² M and 10⁻¹³ M, and any dissociation constant between those listed, including endpoints.

Antigen-binding proteins that have an identical or overlapping epitope will often compete for binding to the antigen (e.g., human GPA). Thus, In some embodiments, there is provided antigen-binding proteins (e.g., antibody or antibody fragment thereof) that compete with the antigen-binding proteins 10F7, Ter119 (in such embodiments wherein the antigen-binding protein cross-reacts with murine GPA), CLB-ery-1 (AME-1), EPR8200, YTH89.1, EPR8199, JC159, GYPA/280, ab40844, HI264, GPHN02, SPM599, GYPA/1725R, ab112201, BRIC 256 or fragments or derivatives thereof. As used herein, “compete” or “competition” shall be given its ordinary meaning, and shall also refer to antigen-binding proteins competing for the same epitope or binding site on a target. Such competition can be determined by an assay in which the reference antigen-binding protein (e.g., antibody or antibody fragment thereof) prevents or inhibits specific binding of a test antigen-binding protein. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay, solid phase direct biotin-avidin EIA, solid phase direct labeled assay, solid phase direct labeled sandwich assay, Luminex, and surface plasmon resonance. Competitive inhibition can be measured by determining the amount of labelled ligand bound to the solid surface or cells in the presence of the test antigen-binding protein. Usually the test antigen-binding protein is present in excess. Antigen-binding proteins or antibodies identified by competition assay (competing antigen-binding proteins or antibodies) include antigen-binding proteins binding to the same epitope as the reference antigen-binding proteins and antigen-binding proteins binding to an adjacent epitope sufficiently proximal to the epitope bound by the reference antigen-binding protein for steric hindrance to occur. Usually, when a competing antigen-binding protein is present in excess, it will inhibit (e.g., reduce) specific binding of a reference antigen-binding protein to a target antigen by at least 40-45%, 45-50%, 50-55%, 55-60%, 60-65%, 65-70%, 70-75% or 75% or more (or any amount of inhibition between those listed. In some embodiments, binding is inhibited by at least 40%, 45%, 50%, 55%, 60%, 65%, 70%, 75%, 80%, 85%, 90%, or 95%. In some embodiments, binding is inhibited by at least 80-85%, 85-90%, 90-95%, 95-97%, or 97% or more, including up to 100% inhibition.

### Nucleic Acids Encoding Antigen-Binding Proteins

The antigen-binding proteins provided herein can be produced by any method known in the art for the synthesis of proteins (e.g., antibodies), in particular, by chemical synthesis or by recombinant expression techniques.

There is provided herein, in several embodiments, nucleic acids encoding the antigen-binding proteins disclosed herein. The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acids may comprise, for example, polynucleotides that encode all or part of an antigen-binding protein, for example, one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length as appropriate for the desired use or function, and can comprise one or more additional sequences, for example, regulatory sequences, and/or be part of a larger nucleic acid, for example, a vector. The skilled artisan will appreciate that, due to the degeneracy of the genetic code, each of the polypeptide sequences disclosed herein is encoded by a large number of other nucleic acid sequences. There is provided herein each degenerate nucleotide sequence encoding each antigen-binding protein disclosed herein.

### Variant Antigen-Binding Proteins

The nucleotide sequences encoding the antigen-binding proteins provided herein can be altered, for example, by random mutagenesis and/or by site-directed mutagenesis (e.g., oligonucleotide-directed site-specific mutagenesis) to create an altered polynucleotide comprising one or more nucleotide substitutions, deletions, and/or insertions as compared to the non-mutated polynucleotide. These and other methods can be used to make, for example, derivatives of the antigen-binding proteins that have a desired property, for example, increased affinity, avidity, or specificity for a desired target, increased activity or stability in vivo or in vitro, or reduced in vivo side-effects as compared to the underivatized antibody. In other embodiments, mutations introduced into the nucleic acids provided herein do not significantly alter the biological activity of a polypeptide that it encodes.

In several embodiments, the antigen-binding proteins provided herein (e.g., an antibody or fragments thereof) may have at least one amino acid substitution, providing that the antigen-binding protein retains the same or better (e.g., higher affinity, lower Kd) desired binding specificity. Therefore, in some embodiments, modifications to the antigen-binding protein structures are provided. In some embodiments, the antigen-binding protein (e.g., but not limited to, an antibody) comprises sequences that each independently differ by 5, 4, 3, 2, 1, or 0 single amino acid additions, substitutions, and/or deletions from a CDR sequence of those set forth in Table 2 herein. These may include amino acid substitutions, which may be conservative or non-conservative that do not destroy the desired binding capability of an antibody. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge, replacement of L-isoform with D-isoform, etc.). In some embodiments, such substituted residues may be introduced into regions of an antigen-binding protein that are homologous with non-human antibodies, or into the non-homologous regions of the molecule.

Non-limiting examples of desired amino acid substitutions are those which: (1) reduce susceptibility to proteolysis, (2) reduce susceptibility to oxidation, (3) alter binding affinity for forming protein complexes, (4) alter binding affinities, and/or (5) confer or modify other physiochemical or functional properties on such polypeptides. For example, in several embodiments, substitutions of amino acids can reduce (or increase) the propensity for post-translational modifications, such as glycosylation. In such embodiments, the degree of post-translational modifications can be tailored to a desired level for a given application. In some embodiments, single or multiple amino acid substitutions (in some embodiments, conservative amino acid substitutions) may be made in the naturally-occurring sequence (in some embodiments, in the portion of the polypeptide outside the domain(s) forming intermolecular contacts). In some embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence).

### Vectors Encoding Antigen-Binding Proteins

There is further provided, in several embodiments, vectors comprising the one or more of the nucleic acids disclosed herein. As used herein, the term “vector” shall be given its ordinary meaning, and shall also refer to a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. In some embodiments, the vectors are capable of autonomous replication in a host cell into which they are introduced (e.g., bacterial vectors comprising a bacterial origin of replication and episomal mammalian vectors). In other embodiments, the vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals).

The recombinant expression vectors provided herein include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). Depending on the embodiment, the design of the expression vector can depend on such factors as the choice of the host cell to be transformed, the level of expression of protein desired, etc. The expression vectors provided herein can be introduced into host cells to thereby produce proteins or peptides, including fusion proteins or peptides, encoded by nucleic acids as described herein.

### Host Cells Expressing Antigen-Binding Proteins

In some embodiments, there are provided host cells into which a recombinant expression vector disclosed herein has been introduced. As used herein, the term “host cell” shall be given its ordinary meaning, and shall also refer to a cell that can be used to express a nucleic acid, e.g., a nucleic acid encoding the antigen-binding proteins disclosed herein. A host cell can be any prokaryotic cell or eukaryotic cell (and/or lysates or transcriptionally and/or translationally active components, e.g., cell-free systems derived from such cells). Prokaryotic host cells include, in some embodiments, gram negative or gram positive organisms, for example E. coli or bacilli. Eukaryotic cells include, in some embodiments, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of suitable mammalian host cell lines include, but are not limited to, Chinese hamster ovary (CHO) cells or their derivatives such as Veggie CHO and related cell lines which grow in serum-free media or CHO strain DXB-11, which is deficient in DHFR. Additional CHO cell lines provided herein include CHO-K1 (ATCC #CCL-61), EM9 (ATCC #CRL-1861), and UV20 (ATCC #CRL-1862). Additional host cells include, but are not limited to, the COS-7 line of monkey kidney cells (ATCC CRL 1651), L cells, C127 cells, 3T3 cells (ATCC CCL 163), AM-1/D cells, HeLa cells, BHK (ATCC CRL 10) cell lines, the CV1/EBNA cell line derived from the African green monkey kidney cell line CV1 (ATCC CCL 70), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells.

In several embodiments, a host cell is a cultured cell that can be transformed or transfected with a polypeptide-encoding nucleic acid, which can then be expressed in the host cell. As used herein, the term “recombinant host cell” shall be given its ordinary meaning, and shall also refer to a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. As used herein, the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein. Vector DNA can be introduced into prokaryotic or eukaryotic cells via conventional transformation or transfection techniques. For stable transfection of mammalian cells, depending upon the expression vector and transfection technique used, only a small fraction of cells may integrate the foreign DNA into their genome. In order to identify and select these integrants, a gene that encodes a selectable marker (e.g., for resistance to antibiotics) may, depending on the embodiment, be introduced into the host cells along with the gene of interest. Additional examples of selectable markers include those which confer resistance to drugs (e.g., G418, hygromycin and methotrexate). Cells stably transfected with the introduced nucleic acid can be identified by drug selection (e.g., cells that have incorporated the selectable marker gene will survive, while the other cells die), among other methods.

In some embodiments, the host cell is co-transfected with two expression vectors, such as, for example, a first vector encoding an antibody heavy chain derived polypeptide and a second vector encoding an antibody light chain derived polypeptide. In some such embodiments, the two vectors may contain identical selectable markers which enable equal expression of heavy and light chain polypeptides. Alternatively, a single vector may be used which encodes, and is capable of expressing, for example, both antibody heavy and light chain polypeptides. In some such embodiments, the light chain is placed before the heavy chain to avoid an excess of toxic free heavy chain. The coding sequences for the heavy and light chains may comprise cDNA or genomic DNA.

Once an antigen-binding protein has been produced by an animal, chemically synthesized, or recombinantly expressed, it may be purified by for example, by chromatography (e.g., ion exchange, affinity, particularly by affinity for the specific antigen after Protein A, and size-exclusion chromatography), centrifugation, differential solubility, or by any other standard technique for the purification of proteins. In addition, the antigen-binding proteins can be fused to heterologous polypeptide sequences described herein or otherwise known to facilitate purification. In some embodiments, there are provided antigen-binding proteins fused or chemically conjugated (including both covalently and non-covalently conjugations) to a polypeptide. Fused or conjugated antigen-binding proteins provided herein may be used for enhanced purification. Moreover, the antigen-binding proteins provided herein can be fused to marker sequences, such as a peptide to facilitate purification. In some embodiments, the marker amino acid sequence is a hexa-histidine peptide, such as the tag provided in a pQE vector, for convenient purification of the fusion protein. Non-limiting examples of other peptide tags useful for purification include, but are not limited to, the “HA” tag, which corresponds to an epitope derived from the influenza hemagglutinin protein and the “flag” tag.

### Conjugates

There is provided herein, in several embodiments, conjugates of an antigen-binding protein provided herein with another molecule. In some embodiments, antigen-binding protein is conjugated to one or more non-proteinaceous chemicals. Non-limiting examples of non-proteinaceous chemicals include, but are not limited to, chemotherapeutic agent, such as cytotoxic agents, cytostatic agents, toxins, and/or radioactive agents. Other derivatives of the antigen-binding proteins provided herein include covalent or aggregative conjugates of the antigen-binding proteins provided herein with other proteins or polypeptides. In some embodiments, the antigen-binding protein is conjugated with one or more therapeutic agents. Non-limiting examples of therapeutic agents suitable for conjugation include, but are not limited to, single-chain variable fragments (scFv), antibody fragments, small molecule drugs, bioactive peptides, bioactive proteins, and/or bioactive biomolecules. There is provided herein, in several embodiments, conjugates of an antigen-binding protein provided herein with an antigenic component, including, but not limited to, a full size antigen, an antigenic and/or immunogenic fragment thereof, or an antigenic and/or immunogenic mimotope thereof.

As used herein, the term conjugated, or the term molecular fusion, shall be given its ordinary meaning, and shall also refer to direct or indirect association by chemical bonds, including, but not limited to, covalent, electrostatic ionic, charge-charge. In some embodiments, the conjugation creates a unit that is sustained by chemical bonding. As used herein, the term direct conjugation shall be given its ordinary meaning, and shall also refer to chemical bonding to the molecule, with or without intermediate linkers or chemical groups. As used herein, indirect conjugation shall be given its ordinary meaning, and shall also refer to chemical linkage to a carrier. In some embodiments, a molecular fusion is formed between an antigen-binding protein disclosed herein and a second polypeptide or protein. In some embodiments, the fusion comprises an antigen-binding protein and another component conjugated directly to each other. In other embodiments, the fusion comprises an antigen-binding protein and another component conjugated indirectly to each other (e.g., through a linker). Non-limiting examples of linkers include, but are not limited to, peptides, polymers, aptamers, nucleic acids, and/or particles. In some embodiments, the particle is a microparticle, a nanoparticle, a polymersome, a liposome, or a micelle. In some embodiments, the polymer is natural or synthetic. In some embodiments, the polymer is linear or branched. A fusion protein that comprises an antigen-binding protein and the second polypeptide is an example of a molecular fusion of the polypeptides, with the fusion protein comprising the polypeptides directly joined to each other or with intervening linker sequences and/or further sequences at one or both ends. The conjugation to the linker may be through covalent or ionic bonds.

In several embodiments, conjugation is accomplished by covalent bonding of the antigen-binding protein to another molecule, with or without use of a linker. The formation of such conjugates is within the skill of artisans and various techniques are known for accomplishing the conjugation, with the choice of the particular technique being guided by the materials to be conjugated. As described further below, the addition of amino acids to the polypeptide (C- or N-terminal) which contain ionizable side chains, i.e. aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, i.e. homo- or hetero-bi-functional PEG.

There is provided herein, in several embodiments, covalent modifications of the antigen-binding proteins. Depending on the embodiment, the covalent modifications are made by chemical synthesis or by enzymatic or chemical cleavage of the antigen-binding proteins in some embodiments. In other embodiments, the covalent modifications of the antigen-binding proteins are introduced into the molecule by reacting targeted amino acid residues of the antibody with an organic derivatizing agent that is capable of reacting with selected side chains or the N- or C-terminal residues.

In some embodiments, cysteinyl residues of the antigen-binding proteins are reacted with alpha-haloacetates (and corresponding amines), such as chloroacetic acid or chloroacetamide, to give carboxymethyl or carboxyamidomethyl derivatives. In some embodiments, iodo-reagents are used. In still further embodiments, cysteinyl residues are derivatized by reaction with bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some embodiments, histidyl residues of the antigen-binding proteins are derivatized by reaction with diethylpyrocarbonate at pH 5.5-7.0 because this agent is relatively specific for the histidyl side chain. In some embodiments, Para-bromophenacyl bromide is used for derivatization; in such embodiments, the reaction is preferably performed in 0.1 M sodium cacodylate at pH 6.0.

In some embodiments, lysyl and amino-terminal residues of the antigen-binding proteins are reacted with succinic or other carboxylic acid anhydrides. In some embodiments, derivatization with these agents has the effect of reversing the charge of the lysinyl residues. Non-limiting examples of other suitable reagents for derivatizing alpha-amino-containing residues and/or e-amino-containing residues include, but are not limited to, imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues of the antigen-binding protein are modified by reaction with one or several conventional reagents, such as, for example, phenylglyoxal, 2,3-butanedione, 1,2-cyclohexanedione, and/or ninhydrin. In some embodiments, derivatization of arginyl residues generally is performed in alkaline conditions due to the high pKa of the guanidine functional group. In such embodiments, these reagents may react with the epsilon-amino groups of lysine as well as the arginine epsilon-amino group.

In some embodiments, specific modification of tyrosyl residues of the antigen-binding protein are conducted. In some such embodiments, spectral labels are introduced into tyrosyl residues by reaction with aromatic diazonium compounds or tetranitromethane. In some embodiments, N-acetylimidizole and tetranitromethane are used to form 0-acetyl tyrosyl species and 3-nitro derivatives, respectively. Tyrosyl residues are iodinated using 1125 or 1131 to prepare labeled proteins for use in radioimmunoassay.

In some embodiments, the carboxyl side groups of the antigen-binding protein (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, in some embodiments, the aspartyl and glutamyl residues are converted to asparaginyl and glutaminyl residues by reaction with ammonium ions.

In some embodiments, glutaminyl and/or asparaginyl residues of the antigen-binding protein are deamidated to the corresponding glutamyl and aspartyl residues, respectively. Depending on the embodiment, these residues are deamidated under neutral or basic conditions. In some embodiments, the antigen-binding protein comprises one or more deamidated forms of these residues.

Other contemplated modifications of the antigen-binding proteins disclosed herein, include, but are not limited to, hydroxylation of proline and lysine, phosphorylation of hydroxyl groups of seryl or threonyl residues, methylation of the alpha-amino groups of lysine, arginine, and histidine side chains, acetylation of the N-terminal amine, and/or amidation of any C-terminal carboxyl group.

In some embodiments, covalent modification of the antigen-binding proteins comprises chemically or enzymatically coupling glycosides to the antibody. In some embodiments, these procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

### Binding to Erythrocyte Cell Surfaces

In some embodiments, the antigen-binding protein binds erythrocytes. In some embodiments, the antigen-binding protein binds erythrocytes of a single species. In some embodiments, the antigen-binding protein binds erythrocytes of one or more species, including, but not limited to, human, cynomolgus macaque, porcine, canine, murine and/or rat. In some embodiments, the antigen-binding protein binds human erythrocytes. In some embodiments, the antigen-binding protein binds cynomolgus erythrocytes. In some embodiments, the antigen-binding protein binds human erythrocytes and cynomolgus erythrocytes. In some embodiments, the antigen-binding protein binds specifically to human erythrocytes and to cynomolgus erythrocytes with similar specificity.

There are provided, in several embodiments, antigen-binding proteins that bind erythrocyte cell surfaces. In some embodiments, the antigen-binding protein binds erythrocyte cell surfaces without altering cell morphology. In some embodiments, the antigen-binding protein binds erythrocyte cell surfaces without cytoplasmic translocation. In some embodiments, the antigen-binding protein binds the erythrocyte cell surfaces without causing apoptosis of bound erythrocytes. In some embodiments, the antigen-binding protein bind erythrocytes with specificity in vivo. In some embodiments, the antigen-binding protein bind erythrocytes with specificity ex vivo. In some embodiments, the antigen-binding protein has a dissociation constant of between about 10 μM and 0.1 nM as determined by equilibrium binding measurements between the antigen-binding protein and erythrocytes

In some embodiments, the antigen-binding protein non-covalently binds erythrocytes. In some embodiments, the antigen-binding protein non-covalently and specifically binds erythrocytes and does not specifically bind to other blood components. In some such embodiments, other blood components are one or more of blood proteins, albumin, fibronectin, platelets, and/or white blood cells. In some such embodiments, other blood components are substantially all components found in a blood sample taken from a typical human. In the context of a blood sample, the term “substantially all” refers to components that are typically present but excludes incidental components in very low concentrations so that they do not effectively reduce the titer of otherwise bioavailable ligands.

### Diagnostic Uses of Antigen-Binding Proteins

In some embodiments, the antigen-binding proteins provided herein are useful for detecting the presence of GPA in a biological sample. The term “detecting” as used herein encompasses quantitative or qualitative detection. In some embodiments, a biological sample comprises a cell or tissue. In some embodiments, there is provided a method of detecting the presence of GPA in a biological sample. In some such embodiments, the method comprises contacting the biological sample with an antigen-binding protein under conditions permissive for binding of an antigen-binding protein to GPA, and detecting whether a complex is formed between the antigen-binding protein and GPA. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In some embodiments, the antigen-binding proteins are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase and bacterial luciferase, luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In some embodiments, antigen-binding proteins are immobilized on an insoluble matrix. Immobilization entails separating the antigen-binding protein disclosed herein from any GPA that remains free in solution. This conventionally is accomplished by either insolubilizing the antigen-binding protein before the assay procedure, as by adsorption to a water-insoluble matrix or surface, or by covalent coupling (for example, using glutaraldehyde cross-linking), or by insolubilizing the antigen-binding protein after formation of a complex between the antigen-binding protein and GPA, e.g., by immunoprecipitation.

### Tolerogenic Compositions

There are provided, in several embodiments, tolerogenic compositions comprising one or more antigen-binding proteins disclosed herein and one or more tolerogenic antigens. In some embodiments, the tolerogenic composition binds specifically to erythrocytes via one or more of the antigen-binding proteins disclosed herein and the tolerogenic antigen is presented to the immune system, thereby inducing antigen-specific tolerance. As used herein, a “tolerogenic antigen” is any substance that serves as a target for the receptors of an immune response (e.g., adaptive immune response), such as the T cell receptor, major histocompatibility complex class I and II, B cell receptor or an antibody. In some embodiments, a tolerogenic antigen may originate from within the body (e.g., “self,” “auto” or “endogenous”). In additional embodiments, a tolerogenic antigen may originate from outside the body (“non-self,” “foreign” or “exogenous”), having entered, for example, by inhalation, ingestion, injection, or transplantation, transdermally, etc. In some embodiments, an exogenous antigen may be biochemically modified in the body. Foreign antigens include, but are not limited to, food antigens, animal antigens, plant antigens, environmental antigens, therapeutic agents, as well as antigens present in an allograft transplant.

In several embodiments, the tolerogenic compositions disclosed herein are used for, for example, treatment of transplant rejection, immune response against a therapeutic agent, autoimmune disease, and food allergy, among other uses.

In several embodiments, the tolerogenic compositions disclosed herein are used to modulate, particularly down-regulate, antigen-specific undesirable immune responses.

In several embodiments, the tolerogenic compositions disclosed herein are used to bind and clear from the circulation specific undesired proteins, including antibodies endogenously generated in a patient (e.g., not exogenous antibodies administered to a patient), peptides and the like, which cause autoimmunity and associated pathologies, allergy, inflammatory immune responses, and anaphylaxis.

According to several embodiments, the provided herein tolerogenic compositions and methods to treat unwanted immune response to self-antigens and foreign antigens, including but not limited to: a foreign transplant antigen against which transplant recipients develop an unwanted immune response (e.g., transplant rejection), a foreign antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease). In several embodiments, therapeutic agents are delivered through the use of, e.g., a gene therapy vector. In some such embodiments, an immune response may be developed against a portion of such vectors and/or their cargo (e.g., the therapeutic agent). Thus, in several embodiments, the antigen to which tolerance is desired comprises a gene therapy vector, including, but not limited to: adenoviruses and adeno-associated virus (and corresponding variants −1, −2, −5, −6, −8, −9, and/or other parvoviruses), lentiviruses, and retroviruses.

Autoimmune disease states that can be treated using the methods and tolerogenic compositions provided herein include, but are not limited to: Acute Disseminated Encephalomyelitis (ADEM); Acute interstitial allergic nephritis (drug allergies); Acute necrotizing hemorrhagic leukoencephalitis; Addison's Disease; Alopecia areata; Alopecia universalis; Ankylosing Spondylitis; Arthritis, juvenile; Arthritis, psoriatic; Arthritis, rheumatoid; Atopic Dermatitis; Autoimmune aplastic anemia; Autoimmune gastritis; Autoimmune hepatitis; Autoimmune hypophysitis; Autoimmune oophoritis; Autoimmune orchitis; Autoimmune polyendocrine syndrome type 1; Autoimmune polyendocrine syndrome type 2; Autoimmune thyroiditis; Behcet's disease; Bronchiolitis obliterans; Bullous pemphigoid; Celiac disease; Churg-Strauss syndrome; Chronic inflammatory demyelinating polyneuropathy; Cicatricial pemphigoid; Crohn's disease; Coxsackie myocarditis; Dermatitis herpetiformis Duhring; Diabetes mellitus (Type 1 diabetes); Erythema nodosum; Epidermolysis bullosa acquisita, Giant cell arteritis (temporal arteritis); Giant cell myocarditis; Goodpasture's syndrome; Graves' disease; Guillain-Barre syndrome; Hashimoto's encephalitis; Hashimoto's thyroiditis; IgG4-related sclerosing disease; Lambert-Eaton syndrome; Mixed connective tissue disease; Mucha-Habermann disease; Multiple sclerosis; Myasthenia gravis; Optic neuritis; Neuromyelitis optica; Parkinson's Disease; Pemphigus vulgaris and variants; Pernicious angemis; Pituitary autoimmune disease; Polymyositis; Postpericardiotomy syndrome; Premature ovarian failure; Primary Biliary Cirrhosis; Primary sclerosing cholangitis; Psoriasis; Rheumatic heart disease; Sjogren's syndrome; Systemic lupus erythematosus; Systemic sclerosis; Ulcerative colitis; Undifferentiated connective tissue disease (UCTD); Uveitis; Vitiligo; and Wegener's granulomatosis.

A particular group of autoimmune disease states that can be treated using the methods and tolerogenic compositions provided herein include, but are not limited to: Acute necrotizing hemorrhagic leukoencephalitis; Addison's Disease; Arthritis, psoriatic; Arthritis, rheumatoid; Autoimmune aplastic anemia; Autoimmune hypophysitis; Autoimmune gastritis; Autoimmune polyendocrine syndrome type 1; Bullous pemphigoid; Celiac disease; Coxsackie myocarditis; Dermatitis herpetiformis Duhring; Diabetes mellitus (Type 1 diabetes); Epidermolysis bullosa acquisita; Giant cell myocarditis; Goodpasture's syndrome; Graves' disease; Hashimoto's thyroiditis; Mixed connective tissue disease; Multiple sclerosis; Myasthenia gravis; Neuromyelitis optica; Parkinson's disease; Pernicious angemis; Pemphigus vulgaris and variants; Pituitary autoimmune disease; Premature ovarian failure; Rheumatic heart disease; Systemic sclerosis; Sjogren's syndrome; Systemic lupus erythematosus; and Vitiligo.

In the embodiments employing an tolerogenic antigen against which an unwanted immune response is developed, such as food antigens, treatment can be provided for reactions against, for example: peanut, apple, milk, egg whites, egg yolks, mustard, celery, shrimp, wheat (and other cereals), strawberry and banana.

According to several embodiments, a patient can be tested to identify a foreign antigen against which an unwanted immune response has developed, and a tolerogenic composition (comprising one or more of the antigen-binding proteins and one or more tolerogenic antigen as disclosed herein) can be developed based on that tolerogenic antigen.

### Testing

Effectiveness in immune modulation can be tested by, for example, measuring the proliferation of OT-I CD8+ cells (transplanted into host mice) in response to the administration of a tolerogenic composition as disclosed herein, as compared with administration of the tolerogenic antigen alone and/or vehicle. According to several embodiments, tolerogenic compositions as disclosed herein, when tested in this manner, show an increase of OT-I cell proliferation as compared with tolerogenic antigen alone or vehicle, demonstrating increased CD8+ T-cell cross-priming, an indicator of induction of immune tolerance. To distinguish T cells being expanded into a functional effector phenotype from those being expanded and deleted, the proliferating OT-I CD8+ T cells can be phenotypically analyzed for molecular signatures of exhaustion [such as programmed death-1 (PD-1), FasL, and others], as well as Annexin-V binding as a hallmark of apoptosis and thus deletion. The OT-I CD8+ T cells can also be assessed for their responsiveness to a tolerogenic antigen challenge with adjuvant in order to demonstrate functional non-responsiveness, and thus immune tolerance, towards the tolerogenic antigen. To do so, the cells are analyzed for inflammatory signatures after administration of tolerogenic compositions into host mice followed by an antigen challenge. According to several embodiments, tolerogenic compositions as disclosed herein, when tested in this manner demonstrate very low (e.g., background) levels of inflammatory OT-I CD8+ T cell responses towards OVA in comparison to control groups, thus demonstrating immune tolerance.

According to several embodiments, humoral immune response can be tested by administering a tolerogenic composition as disclosed herein incorporating one or more tolerogenic antigens as disclosed herein as compared with the administration of the tolerogenic antigen alone or just vehicle, and measuring the levels of resulting antibodies. According to several embodiments, tolerogenic compositions of the disclosure when tested in this manner show very low (e.g., background) levels of antibody formation responsive to their administration and the administration of vehicle, with significantly higher levels of antibody formation responsive to administration of the tolerogenic antigen alone. The reduced antibody formation is an indicator of induction of immune tolerance.

Effectiveness in tolerization against a tolerogenic antigen can be tested as above with reference to humoral immune response, where several weeks following treatment(s) with a tolerogenic composition as disclosed herein, a group of subjects is challenged by administration of the tolerogenic antigen alone, followed by measuring the levels of antibodies to the tolerogenic antigen. According to several embodiments, tolerogenic compositions of the disclosure when tested in this manner show low levels of antibody formation responsive to challenge with the tolerogenic antigen in groups pretreated with such tolerogenic compositions as compared to groups that are not pretreated.

Disease-focused experimental models include, but are not limited to, the NOD (or non-obese diabetic) mouse model of type-1 diabetes autoimmunity and tolerance and the EAE (experimental autoimmune encephalomyelitis) model for the human inflammatory demyelinating disease, multiple sclerosis. According to several embodiments, such models can be used to demonstrate the effective induction of tolerance using the tolerogenic compositions as disclosed herein.

### Administration

According to several embodiments, tolerogenic compositions of the disclosure are administered at a therapeutically effective dosage, e.g., a dosage sufficient to provide treatment for the disease states previously described. Administration of the tolerogenic compounds of the disclosure or the pharmaceutically acceptable salts thereof can be via any of the accepted modes of administration for agents that serve similar utilities.

While human dosage levels have yet to be optimized for the tolerogenic compounds of the disclosure, these can initially be extrapolated from the about 10 μg to 100 μg doses administered for mice. Generally, an individual human dose is from about 0.01 to 20.0 mg/kg of body weight, preferably about 0.1 to 10 mg/kg of body weight, and most preferably about 0.3 to 2.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered may be dependent on any or all of the following: the subject and disease state being treated, the severity of the affliction, the manner and schedule of administration and the judgment of the prescribing physician. It will also be appreciated that amounts administered may depend upon the molecular weight of the tolerogenic antigen, antibody, antibody fragment (or other antigen-binding fragment described herein) as well as the size of the linker.

The tolerogenic compositions of the disclosure can be administered either alone or in combination with other pharmaceutically acceptable excipients. Typical routes of administration that are used, depending on the embodiment include, but are not limited to, oral, topical, transdermal, injection (intramuscular, intravenous, or intra-arterial)) and the like. Depending on the embodiment, the formulations optionally include a conventional pharmaceutical carrier or excipient and a tolerogenic composition of the disclosure or a pharmaceutically acceptable salt thereof. In addition, these tolerogenic compositions can include other medicinal agents, pharmaceutical agents, carriers, and the like, including, but not limited to the therapeutic protein, peptide, antibody or antibody-like molecule corresponding to the antigen employed in the tolerogenic composition of the disclosure, and other active agents that can act as immune-modulating agents and more specifically can have inhibitory effects on B-cells, including anti-folates, immune suppressants, cyostatics, mitotic inhibitors, and anti-metabolites, or combinations thereof.

Generally, depending on the intended mode of administration, the pharmaceutically acceptable composition will contain about 0.1% to 95%, by weight of a tolerogenic composition of the disclosure, the remainder being suitable pharmaceutical excipients, carriers, etc. For example, in several embodiments, a pharmaceutically acceptable composition comprises a tolerogenic composition as provided for herein in an amount of about 0.1% to about 1%, about 1% to about 5%, about 5% to about 15%, about 15% to about 25%, about 25% to about 50%, about 50% to about 75%, about 75% to about 95% by weight (including any amount between those ranges listed, including endpoints). Dosage forms or compositions containing active ingredient in the range of 0.005% to 95% with the balance made up from non-toxic carrier can be prepared. For example, in several embodiments, dosage forms may comprise an active ingredient in an amount of about 0.005% to about 0.01%, about 0.01% to about 0.05%, about 0.05% to about 0.1%, about 0.1% to about 1%, about 1% to about 5%, about 5% to about 15%, about 15% to about 25%, about 25% to about 50%, about 50% to about 75%, about 75% to about 95% (including any amount between those ranges listed, including endpoints).

Liquid pharmaceutically administrable tolerogenic compositions can, for example, be prepared by dissolving, dispersing, etc. an active tolerogenic composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands.

### Tolerogenic Antigens

The tolerogenic antigens employed in a tolerogenic composition comprising the antigen-binding proteins disclosed herein can be a protein or a peptide, e.g. the tolerogenic antigen may be a complete or partial therapeutic agent, a full-length transplant protein or peptide thereof, a full-length autoantigen or peptide thereof, a full-length allergen or peptide thereof, and/or a nucleic acid, or a mimetic of an aforementioned tolerogenic antigen. A listing of any particular tolerogenic antigen in a category or association with any particular disease or reaction does not preclude that tolerogenic antigen from being considered part of another category or associated with another disease or reaction.

Tolerogenic antigens employed in the practice of the present disclosure can be one or more of the following,
Therapeutic agents that are proteins, peptides, antibodies and antibody-like molecules, including antibody fragments and fusion proteins with antibodies and antibody fragments, and gene therapy vectors. These include human, non-human (such as mouse) and non-natural (i.e., engineered) proteins, antibodies, chimeric antibodies, humanized antibodies, viruses and virus-like particles, and non-antibody binding scaffolds, such as fibronectins, DARPins, knottins, and the like.
Human allograft transplantation antigens against which transplant recipients develop an unwanted immune response.
Self-antigens that cause an unwanted, autoimmune response. While self-antigens are of an endogenous origin in patient (e.g., an autoimmune patient), the polypeptides employed in the disclosed tolerogenic compositions may, depending on the embodiment, be synthesized exogenously (as opposed to being purified and concentrated from a source of origin).
Foreign antigens, such as food, animal, plant and environmental antigens, against which a patient experiences an unwanted immune response. While a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, considered foreign antigens. In the interest of simplicity no attempt will be made to describe distinguish and define all of such potentially overlapping groups, as those skilled in the art can appreciate the tolerogenic antigens that can be employed in the tolerogenic compositions of the disclosure, particularly in light of the detailed description and examples.

The tolerogenic antigen can be a complete protein, a portion of a complete protein, a peptide, or the like, and can be derivatized (as discussed above) for attachment to a linker and/or antigen-binding moiety, can be a variant and/or can contain conservative substitutions, particularly maintaining sequence identity, and/or can be desialylated.

In the embodiments where the tolerogenic antigen is a therapeutic protein, peptide, antibody or antibody-like molecule, specific tolerogenic antigens include, but are not limited to: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

Particular therapeutic protein, peptide, antibody or antibody-like molecules include, but are not limited to, Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Some embodiments, utilize hemostatic agents (e.g., Factor VIII and IX), Insulin (including rHu Insulin), and the non-human therapeutics uricase, PAL and asparaginase.

In several embodiments, therapeutic agents are delivered through the use of, e.g., a gene therapy vector. In some such embodiments, an immune response may be developed against a portion of such vectors and/or their cargo (e.g., the therapeutic agent). Thus, in several embodiments, the antigen to which tolerance is desired comprises a gene therapy vector, including, but are not limited to: adenoviruses and adeno-associated virus (and corresponding variants −1, −2, −5, −6, −8, −9, and/or other parvoviruses), lentiviruses, and retroviruses.

Unwanted immune response in hematology and transplant includes autoimmune aplastic anemia, transplant rejection (generally), and Graft vs. Host Disease (bone marrow transplant rejection). In the embodiments where the tolerogenic antigen is a human allograft transplantation antigen, specific sequences can be selected from: subunits of the various MHC class I and MHC class II haplotype proteins (for example, donor/recipient differences identified in tissue cross-matching), and single-amino-acid polymorphisms on minor blood group antigens including RhCE, Kell, Kidd, Duffy and Ss. Such tolerogenic compositions can be prepared individually for a given donor/recipient pair.

In the embodiments where the tolerogenic antigen is a self-antigen, specific tolerogenic antigens (and the autoimmune disease with which they are associated) can be selected from:

In type 1 diabetes mellitus, antigens include, but are not limited to: insulin, proinsulin, preproinsulin, glutamic acid decarboxylase-65 (GAD-65 or glutamate decarboxylase 2), GAD-67, glucose-6 phosphatase 2 (IGRP or islet-specific glucose 6 phosphatase catalytic subunit related protein), insulinoma-associated protein 2 (IA-2), and insulinoma-associated protein 2β (IA-2β); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5, or immunogenic fragments or portions of any of such antigens. It should be noted that insulin is an example of an antigen that can be characterized both as a self-antigen and a therapeutic protein antigen. For example, rHu Insulin and bovine insulin are therapeutic protein antigens (that are the subject of unwanted immune attack), whereas endogenous human insulin is a self-antigen (that is the subject of an unwanted immune attack). Because endogenous human insulin is not available to be employed in a pharmaceutical composition, a recombinant form is employed in certain embodiments of the tolerogenic compositions of the disclosure.

In several embodiments, human insulin, including an exogenously obtained form useful in the tolerogenic compositions of the disclosure, has the following sequence (UNIPROT P01308):

(SEQ ID NO: 163)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN.

In several embodiments, GAD-65, including an exogenously obtained form useful in the tolerogenic compositions of the disclosure, has the following sequence (UNIPROT Q05329):

(SEQ ID NO: 164)
MASPGSGFWSFGSEDGSGDSENPGTARAWCQVAQKFTGGIGNKLCALLYG
DAEKPAESGGSQPPRAAARKAACACDQKPCSCSKVDVNYAFLHATDLLPA
CDGERPTLAFLQDVMNILLQYVVKSFDRSTKVIDFHYPNELLQEYNWELA
DQPQNLEEILMHCQTTLKYAIKTGHPRYFNQLSTGLDMVGLAADWLTSTA
NTNMFTYEIAPVFVLLEYVTLKKMREIIGWPGGSGDGIFSPGGAISNMYA
MMIARFKMFPEVKEKGMAALPRLIAFTSEHSHFSLKKGAAALGIGTDSVI
LIKCDERGKMIPSDLERRILEAKQKGFVPFLVSATAGTTVYGAFDPLLAV
ADICKKYKIWMHVDAAWGGGLLMSRKHKWKLSGVERANSVTWNPHKMMGV
PLQCSALLVREEGLMQNCNQMHASYLFQQDKHYDLSYDTGDKALQCGRHV
DVFKLWLMWRAKGTTGFEAHVDKCLELAEYLYNIIKNREGYEMVFDGKPQ
HTNVCFWYIPPSLRTLEDNEERMSRLSKVAPVIKARMMEYGTTMVSYQPL
GDKVNFFRMVISNPAATHQDIDFLIEEIERLGQDL.

In several embodiments, IGRP, including an exogenously obtained from useful in the tolerogenic compositions of the disclosure, has the following sequence (UNIPROT QN9QR9):

(SEQ ID NO: 165)
MDFLHRNGVLIIQHLQKDYRAYYTFLNFMSNVGDPRNIFFIYFPLCFQFN
QTVGTKMIWVAVIGDWLNLIFKWILFGHRPYWWVQETQIYPNHSSPCLEQ
FPTTCETGPGSPSGHAMGASCVWYVMVTAALSHTVCGMDKFSITLHRLTW
SFLWSVFWLIQISVCISRVFIATHFPHQVILGVIGGMLVAEAFEHTPGIQ
TASLGTYLKTNLFLFLFAVGFYLLLRVLNIDLLWSVPIAKKWCANPDWIH
IDTTPFAGLVRNLGVLFGLGFAINSEMFLLSCRGGNNYTLSFRLLCALTS
LTILQLYHFLQIPTHEEHLFYVLSFCKSASIPLTVVAFIPYSVHMLMKQS
GKKSQ.

In several embodiments, human proinsulin, including an exogenously obtained from useful in the tolerogenic compositions of the disclosure, has the following sequence:

(SEQ ID NO: 203)
FVNQHLCGSHLVEALYLVCGERGFFYTPKTRREAEDLQVGQVELGGGPGA
GSLQPLALEGSLQKRGIVEQCCTSICSLYQLENYCN.

Depending on the embodiment, peptides/epitopes useful in the tolerogenic compositions of the disclosure for treating type 1 diabetes include some or all of the following sequences, individually in a tolerogenic composition or together in a cocktail of tolerogenic compositions:

Human Proinsulin 1-70:
(SEQ ID NO: 204)
FVNQHLCGSHLVEALYLVCGERGFFYTPKTRREAEDLQVGQVELGGGPG
AGSLQPLALEGSLQKRGIVEQ;
Human Proinsulin 9-70:
(SEQ ID NO: 205)
SHLVEALYLVCGERGFFYTPKTRREAEDLQVGQVELGGGPGAGSLQPLA
LEGSLQKRGIVEQ;
Human Proinsulin 9-38:
(SEQ ID NO: 206)
SHLVEALYLVCGERGFFYTPKTRREAEDLQ;
Human Proinsulin 1-38:
(SEQ ID NO: 207)
FVNQHLCGSHLVEALYLVCGERGFFYTPKTRREAEDLQ;
Human Proinsulin 9-23:
(SEQ ID NO: 208)
SHLVEALYLVCGERG;
Human Proinsulin 45-71 (C13-A6):
(SEQ ID NO: 209)
GGGPGAGSLQPLALEGSLQKRGIVEQC;
Human Proinsulin C24-A1:;
(SEQ ID NO: 210)
LALEGSLQKRG
Human Proinsulin C19-A3:
(SEQ ID NO: 211)
GSLQPLALEGSLQKRGIV;
Human Proinsulin C13-32:
(SEQ ID NO: 212)
GGGPGAGSLQPLALEGSLQK;
Human Proinsulin B9-C4:
(SEQ ID NO: 213)
SHLVEALYLVCGERGFFYTPKTRREAED;
Human Proinsulin C22-A5:
(SEQ ID NO: 214)
QPLALEGSLQKRGIVEQ;

In autoimmune diseases of the thyroid, including Hashimoto's thyroiditis and Graves' disease, main antigens include, but are not limited to, thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor.

In Addison's Disease, main antigens include, but are not limited to, 21-hydroxylase, 17α-hydroxylase, and P450 side chain cleavage enzyme (P450scc); other antigens include ACTH receptor, P450c21 and P450c17.

In premature ovarian failure, main antigens include, but are not limited to, FSH receptor and α-enolase.

In autoimmune hypophysitis, or pituitary autoimmune disease, main antigens include, but are not limited to, pituitary gland-specific protein factor (PGSF) 1a and 2; another antigen is type 2 iodothyronine deiodinase.

In multiple sclerosis, main antigens include, but are not limited to, myelin basic protein (“MBP”), myelin oligodendrocyte glycoprotein (“MOG”) and myelin proteolipid protein (“PLP”).

MBP, including an exogenously obtained from useful in the tolerogenic compositions of the disclosure, has the following sequence (UNIPROT P02686):

(SEQ ID NO: 166)
MGNHAGKRELNAEKASTNSETNRGESEKKRNLGELSRTTSEDNEVFGEAD
ANQNNGTSSQDTAVTDSKRTADPKNAWQDAHPADPGSRPHLIRLFSRDAP
GREDNTFKDRPSESDELQTIQEDSAATSESLDVMASQKRPSQRHGSKYLA
TASTMDHARHGFLPRHRDTGILDSIGRFFGGDRGAPKRGSGKDSHHPART
AHYGSLPQKSHGRTQDENPVVHFFKNIVTPRTPPPSQGKGRGLSLSRFSW
GAEGQRPGFGYGGRASDYKSAHKGFKGVDAQGTLSKIFKLGGRDSRSGSP
MARR.

MOG, including an exogenously obtained from useful in the tolerogenic compositions of the disclosure, has the following sequence (UNIPROT Q16653):

(SEQ ID NO: 167)
MASLSRPSLPSCLCSFLLLLLLQVSSSYAGQFRVIGPRHPIRALVGDEVEL
PCRISPGKNATGMEVGWYRPPFSRVVHLYRNGKDQDGDQAPEYRGRTELLK
DAIGEGKVTLRIRNVRFSDEGGFTCFFRDHSYQEEAAMELKVEDPFYWVSP
GVLVLLAVLPVLLLQITVGLIFLCLQYRLRGKLRAEIENLHRTFDPHFLRV
PCWKITLFVIVPVLGPLVALIICYNWLHRRLAGQFLEELRNPF.

PLP, including an exogenously obtained from useful in the tolerogenic compositions of the disclosure, has the following sequence (UNIPROT P60201):

(SEQ ID NO: 168)
MGLLECCARCLVGAPFASLVATGLCFFGVALFCGCGHEALTGTEKLIETYF
SKNYQDYEYLINVIHAFQYVIYGTASFFFLYGALLLAEGFYTTGAVRQIFG
DYKTTICGKGLSATVTGGQKGRGSRGQHQAHSLERVCHCLGKWLGHPDKFV
GITYALTVVWLLVFACSAVPVYIYFNTWTTCQSIAFPSKTSASIGSLCADA
RMYGVLPWNAFPGKVCGSNLLSICKTAEFQMTFHLFIAAFVGAAATLVSLL
TFMIAATYNFAVLKLMGRGTKF.

Peptides/epitopes useful in the tolerogenic compositions of the disclosure for treating multiple sclerosis include some or all of the following sequences, individually in a tolerogenic composition or together in a combination (e.g., cocktail) of tolerogenic compositions of 2-5, 5-10, 10-15, 15-20 (and overlapping ranges therein) or more of the following:

MBP13-32:
(SEQ ID NO: 169)
KYLATASTMDHARHGFLPRH;
MBP83-99:
(SEQ ID NO: 170)
ENPWHFFKNIVTPRTP;
MBP111-129:
(SEQ ID NO: 171)
LSRFSWGAEGQRPGFGYGG;
MBP146-170:
(SEQ ID NO: 172)
AQGTLSKIFKLGGRDSRSGSPMARR;
MOG1-20:
(SEQ ID NO: 173)
GQFRVIGPRHPIRALVGDEV;
MOG35-55:
(SEQ ID NO: 174)
MEVGWYRPPFSRWHLYRNGK;
and
PLP139-154:
(SEQ ID NO: 175)
HCLGKWLGHPDKFVGI.
MOG35-55:
(SEQ ID NO: 186)
MEVGWYRSPFSRVVHLYRNGK
MOG30-60:
(SEQ ID NO: 187)
KNATGMEVGWYRSPFSRVVHLYRNGKDQDAE
MBP83-99:
(SEQ ID NO: 188)
ENPVVHFFKNIVTPRTP
MOG35-55:
(SEQ ID NO: 189)
MEVGWYRPPFSRVVHLYRNGK
MBP82-98:
(SEQ ID NO: 190)
DENPVVHFFKNIVTPRT
MBP82-99:
(SEQ ID NO: 191)
DENPVVHFFKNIVTPRTP
MBP82-106:
(SEQ ID NO: 192)
DENPVVHFFKNIVTPRTPPPSQGKG
MBP87-106:
(SEQ ID NO: 193)
VHFFKNIVTPRTPPPSQGKG
MBP131-155:
(SEQ ID NO: 194)
ASDYKSAHKGLKGVDAQGTLSKIFK
PLP41-58:
(SEQ ID NO: 195)
GTEKLIETYFSKNYQDYE
PLP89-106:
(SEQ ID NO: 196)
GFYTTGAVRQIFGDYKTT
PLP95-116:
(SEQ ID NO: 197)
AVRQIFGDYKTTICGKGLSATV
PLP178-197:
(SEQ ID NO: 198)
NTWTTCQSIAFPSKTSASIG
PLP190-209:
(SEQ ID NO: 199)
SKTSASIGSLCADARMYGVL
MOG11-30:
(SEQ ID NO: 200)
PIRALVGDEVELPCRISPGK
MOG21-40:
(SEQ ID NO: 201)
ELPCRISPGKNATGMEVGWY
MOG64-86:
(SEQ ID NO: 202)
EYRGRTELLKDAIGEGKVTLRIR

In rheumatoid arthritis, main antigens include, but are not limited to, collagen II, immunoglobulin binding protein, the fragment crystallizable region of immunoglobulin G, double-stranded DNA, and the natural and cirtullinated forms of proteins implicated in rheumatoid arthritis pathology, including fibrin/fibrinogen, vimentin, collagen I and II, and alpha-enolase.

In autoimmune gastritis, a main antigen is H+,K+-ATPase.

In pernicious angemis, a main antigen is intrinsic factor.

In celiac disease, main antigens include, but are not limited to, tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that while the main antigen of celiac disease is alpha gliadin, alpha gliadin turns more immunogenic in the body through deamidation by tissue glutaminase converting alpha gliadin's glutamines to glutamic acid. Thus, while alpha gliadin is originally a foreign food antigen, once it has been modified in the body to become more immunogenic it can be characterized as a self-antigen, depending on the embodiment.

In vitiligo, a main antigen is tyrosinase, and tyrosinase related protein 1 and 2.

MART1, Melanoma antigen recognized by T cells 1, Melan-A, including an exogenously obtained from useful in the tolerogenic compositions of the disclosure, has the following sequence (UNIPROT Q16655):

(SEQ ID NO: 176)
MPREDAHFIYGYPKKGHGHSYTTAEEAAGIGILTVILGVLLLIGCWYCRRR
NGYRALMDKSLHVGTQCALTRRCPQEGFDHRDSKVSLQEKNCEPVVPNAPP
AYEKLSAEQSPPPYSP.

Tyrosinase, including an exogenously obtained from useful in the tolerogenic compositions of the disclosure, has the following sequence (UNIPROT P14679):

(SEQ ID NO: 177)
MLLAVLYCLLWSFQTSAGHFPRACVSSKNLMEKECCPPWSGDRSPCGQLSG
RGSCQNILLSNAPLGPQFPFTGVDDRESWPSVFYNRTCQCSGNFMGFNCGN
CKFGFWGPNCTERRLLVRRNIFDLSAPEKDKFFAYLTLAKHTISSDYVIPI
GTYGQMKNGSTPMFNDINIYDLFVWMHYYVSMDALLGGSEIWRDIDFAHEA
PAFLPWHRLFLLRWEQEIQKLTGDENFTIPYWDWRDAEKCDICTDEYMGGQ
HPTNPNLLSPASFFSSWQIVCSRLEEYNSHQSLCNGTPEGPLRRNPGNHDK
SRTPRLPSSADVEFCLSLTQYESGSMDKAANFSFRNTLEGFASPLTGIADA
SQSSMHNALHIYMNGTMSQVQGSANDPIFLLHHAFVDSIFEQWLRRHRPLQ
EVYPEANAPIGHNRESYMVPFIPLYRNGDFFISSKDLGYDYSYLQDSDPDS
FQDYIKSYLEQASRIWSWLLGAAMVGAVLTALLAGLVSLLCRHKRKQLPEE
KQPLLMEKEDYHSLYQSHL.

Melanocyte protein PMEL, gp100, including an exogenously obtained form useful in the tolerogenic compositions of the disclosure, has the following sequence (UNIPROT P40967):

(SEQ ID NO: 178)
MDLVLKRCLLHLAVIGALLAVGATKVPRNQDWLGVSRQLRTKAWNRQLYPE
WTEAQRLDCWRGGQVSLKVSNDGPTLIGANASFSIALNFPGSQKVLPDGQV
IWVNNTIINGSQVWGGQPVYPQETDDACIFPDGGPCPSGSWSQKRSFVYVW
KTWGQYWQVLGGPVSGLSIGTGRAMLGTHTMEVTVYHRRGSRSYVPLAHSS
SAFTITDQVPFSVSVSQLRALDGGNKHFLRNQPLTFALQLHDPSGYLAEAD
LSYTWDFGDSSGTLISRALVVTHTYLEPGPVTAQVVLQAAIPLTSCGSSPV
PGTTDGHRPTAEAPNTTAGQVPTTEVVGTTPGQAPTAEPSGTTSVQVPTTE
VISTAPVQMPTAESTGMTPEKVPVSEVMGTTLAEMSTPEATGMTPAEVSIV
VLSGTTAAQVTTTEWVETTARELPIPEPEGPDASSIMSTESITGSLGPLLD
GTATLRLVKRQVPLDCVLYRYGSFSVTLDIVQGIESAEILQAVPSGEGDAF
ELTVSCQGGLPKEACMEISSPGCQPPAQRLCQPVLPSPACQLVLHQILKGG
SGTYCLNVSLADTNSLAVVSTQLIMPGQEAGLGQVPLIVGILLVLMAVVLA
SLIYRRRLMKQDFSVPQLPHSSSHWLRLPRIFCSCPIGENSPLLSGQQV.

In myasthenia gravis, a main antigen is acetylcholine receptor.

In pemphigus vulgaris and variants, main antigens include, but are not limited to, desmoglein 3, 1 and 4; other antigens include pemphaxin, desmocollins, plakoglobin, perplakin, desmoplakins, and acetylcholine receptor.

In bullous pemphigoid, main antigens include, but are not limited to, BP180 and BP230; other antigens include plectin and laminin 5.

In dermatitis herpetiformis Duhring, main antigens include, but are not limited to, endomysium and tissue transglutaminase.

In epidermolysis bullosa acquisita, a main antigen is collagen VII.

In systemic sclerosis, main antigens include, but are not limited to, matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23.

In mixed connective tissue disease, a main antigen is U1snRNP.

In Sjogren's syndrome, the main antigens include, but are not limited to, nuclear antigens SS-A and SS-B; other antigens include fodrin, poly(ADP-ribose) polymerase and topoisomerase, muscarinic receptors, and the Fc-gamma receptor IIIb.

In systemic lupus erythematosus, main antigens include, but are not limited to, nuclear proteins including the “Smith antigen,” SS-A, high mobility group box 1 (HMGB1), nucleosomes, histone proteins and double-stranded DNA (against which auto-antibodies are made in the disease process).

In Goodpasture's syndrome, main antigens include, but are not limited to, glomerular basement membrane proteins including collagen IV.

In rheumatic heart disease, a main antigen is cardiac myosin.

In autoimmune polyendocrine syndrome type 1 antigens include, but are not limited to, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In neuromyelitis optica, a main antigen is AQP4.

Aquaporin-4, including an exogenously obtained form useful in the tolerogenic compositions of the disclosure, has the following sequence (UNIPROT P55087):

(SEQ ID NO: 179)
MSDRPTARRWGKCGPLCTRENIMVAFKGVWTQAFWKAVTAEFLAMLIFVLL
SLGSTINWGGTEKPLPVDMVLISLCFGLSIATMVQCFGHISGGHINPAVTV
AMVCTRKISIAKSVFYIAAQCLGAIIGAGILYLVTPPSVVGGLGVTMVHGN
LTAGHGLLVELIITFQLVFTIFASCDSKRTDVTGSIALAIGFSVAIGHLFA
INYTGASMNPARSFGPAVIMGNWENHWIYWVGPIIGAVLAGGLYEYVFCPD
VEFKRRFKEAFSKAAQQTKGSYMEVEDNRSOVETDDLILKPGVVHVIDVDR
GEEKKGKDOSGEVLSSV.

In uveitis, main antigens include, but are not limited to, Retinal S-antigen or “5-arrestin” and interphotoreceptor retinoid binding protein (IRBP) or retinol-binding protein 3.

S-arrestin, including an exogenously obtained form useful in the tolerogenic compositions of the disclosure, has the following sequence (UNIPROT P10523):

(SEQ ID NO: 180)
MAASGKTSKSEPNHVIFKKISRDKSVTIYLGNRDYIDHVSQVQPVDGVVLV
DPDLVKGKKVYVTLTCAFRYGQEDIDVIGLTFRRDLYFSRVQVYPPVGAAS
TPTKLQESLLKKLGSNTYPFLLTFPDYLPCSVMLQPAPQDSGKSCGVDFEV
KAFATDSTDAEEDKIPKKSSVRLLIRKVQHAPLEMGPQPRAEAAWQFFMSD
KPLHLAVSLNKEIYFHGEPIPVTVTVTNNTEKTVKKIKAFVEQVANVVLYS
SDYYVKPVAMEEAQEKVPPNSTLTKTLTLLPLLANNRERRGIALDGKIKHE
DTNLASSTIIKEGIDRTVLGILVSYQIKVKLTVSGFLGELTSSEVATEVPF
RLMHPQPEDPAKESYQDANLVFEEFARHNLKDAGEAEEGKRDKNDVDE.

IRBP, including an exogenously obtained form useful in the tolerogenic compositions of the disclosure, has the following sequence (UNIPROT P10745):

(SEQ ID NO: 181)
MMREWVLLMSVLLCGLAGPTHLFQPSLVLDMAKVLLDNYCFPENLLGMQEA
IQQAIKSHEILSISDPQTLASVLTAGVQSSLNDPRLVISYEPSTPEPPPQV
PALTSLSEEELLAWLQRGLRHEVLEGNVGYLRVDSVPGQEVLSMMGEFLVA
HVWGNLMGTSALVLDLRHCTGGQVSGIPYIISYLHPGNTILHVDTIYNRPS
NTTTEIWTLPQVLGERYGADKDVVVLTSSQTRGVAEDIAHILKQMRRAIVV
GERTGGGALDLRKLRIGESDFFFTVPVSRSLGPLGGGSQTWEGSGVLPCVG
TPAEQALEKALAILTLRSALPGVVHCLQEVLKDYYTLVDRVPTLLQHLASM
DFSTVVSEEDLVTKLNAGLQAASEDPRLLVRAIGPTETPSWPAPDAAAEDS
PGVAPELPEDEAIRQALVDSVFQVSVLPGNVGYLRFDSFADASVLGVLAPY
VLRQVWEPLQDTEHLIMDLRHNPGGPSSAVPLLLSYFQGPEAGPVHLFTTY
DRRTNITQEHFSHMELPGPRYSTQRGVYLLTSHRTATAAEEFAFLMQSLGW
ATLVGEITAGNLLHTRTVPLLDTPEGSLALTVPVLTFIDNHGEAWLGGGVV
PDAIVLAEEALDKAQEVLEFHQSLGALVEGTGHLLEAHYARPEVVGQTSAL
LRAKLAQGAYRTAVDLESLASQLTADLQEVSGDHRLLVFHSPGELVVEEAP
PPPPAVPSPEELTYLIEALFKTEVLPGQLGYLRFDAMAELETVKAVGPQLV
RLVWQQLVDTAALVIDLRYNPGSYSTAIPLLCSYFFEAEPRQHLYSVFDRA
TSKVTEVWTLPQVAGQRYGSHKDLYILMSHTSGSAAEAFAHTMQDLQRATV
IGEPTAGGALSVGIYQVGSSPLYASMPTQMAMSATTGKAWDLAGVEPDITV
PMSEALSIAQDIVALRAKVPTVLQTAGKLVADNYASAELGAKMATKLSGLQ
SRYSRVTSEVALAEILGADLQMLSGDPHLKAAHIPENAKDRIPGIVPMQIP
SPEVFEELIKFSFHTNVLEDNIGYLRFDMFGDGELLTQVSRLLVEHIWKKI
MHTDAMIIDMRFNIGGPTSSIPILCSYFFDEGPPVLLDKIYSRPDDSVSEL
WTHAQVVGERYGSKKSMVILTSSVTAGTAEEFTYIMKRLGRALVIGEVTSG
GCQPPQTYHVDDTNLYLTIPTARSVGASDGSSWEGVGVTPHVVVPAEEALA
RAKEMLQHNQLRVKRSPGLQDHL.

In the embodiments where the tolerogenic antigen is a foreign antigen against which an unwanted immune response can be developed, such as food antigens, specific antigens include, but are not limited to:
from peanut: conarachin (Ara h 1), allergen II (Ara h 2), arachis agglutinin, conglutin (Ara h 6);
   conarachin, for example has the sequence identified as UNIPROT Q6PSU6
from apple: 31 kda major allergen/disease resistance protein homolog (Mal d 2), lipid transfer protein precursor (Mal d 3), major allergen Mal d 1.03D (Mal d 1);
from milk: α-lactalbumin (ALA), lactotransferrin; from kiwi: actinidin (Act c 1, Act d 1), phytocystatin, thaumatin-like protein (Act d 2), kiwellin (Act d 5);
from egg whites: ovomucoid, ovalbumin, ovotransferrin, and lysozyme;
from egg yolks: livetin, apovitillin, and vosvetin;
from mustard: 2S albumin (Sin a 1), 11S globulin (Sin a 2), lipid transfer protein (Sin a 3), profilin (Sin a 4);
from celery: profilin (Api g 4), high molecular weight glycoprotein (Api g 5);
from shrimp: Pen a 1 allergen (Pen a 1), allergen Pen m 2 (Pen m 2), tropomyosin fast isoform;
from wheat and/or other cereals: high molecular weight glutenin, low molecular weight glutenin, alpha-, gamma- and omega-gliadin, hordein, secalin and/or avenin;
   peptides/epitopes useful in the tolerogenic compositions of the disclosure for treating Celiac Disease include some or all of the following sequences, individually in a combination (e.g., cocktail) of tolerogenic compositions of 2-5, 5-10, 10-15, 15-20 (and overlapping ranges therein) or more of the following:
      DQ-2 relevant, Alpha-gliadin “33-mer” native:

(SEQ ID NO: 182)
LQLQPFPQPQLPYPQPQLPYPQPQLPYPQPQPF

DQ-2 relevant, Alpha-gliadin “33-mer” deamidated:

(SEQ ID NO: 183)
LQLQPFPQPELPYPQPELPYPQPELPYPQPQPF

DQ-8 relevant, Alpha-gliadin:

(SEQ ID NO: 184)
QQYPSGQGSFQPSQQNPQ

DQ-8 relevant, Omega-gliadin (wheat, U5UA46):

(SEQ ID NO: 185)
QPFPQPEQPFPW

Alpha-gliadin “15-mer” fragment:

(SEQ ID NO: 215)
ELQPFPQPELPYPQP

from strawberry: major strawberry allergy Fra a 1-E (Fra a 1); and
from banana: profilin (Mus xp 1).

In Parkinson's disease, the main antigen is alpha synuclein. Alpha synuclein, including an exogenously obtained form useful in the tolerogenic compositions of the disclosure, has the following sequence (UNIPROT P37840):

(SEQ ID NO: 216)
MDVFMKGLSKAKEGVVAAAEKTKQGVAEAAGKTKEGVLYVGSKTKEGVVHG
VATVAEKTKEQVTNVGGAVVTGVTAVAQKTVEGAGSIAAATGFVKKDQLGK
NEEGAPQEGILEDMPVDPDNEAYEMPSEEGYQDYEPEA.

In the embodiments where the tolerogenic antigen is a foreign antigen against which an unwanted immune response is developed, such as to animal, plant and environmental antigens, specific antigens can, for example, be: cat, mouse, dog, horse, bee, dust, tree and goldenrod, including the following proteins or peptides derived from:
weeds, (including ragweed allergens amb a 1, 2, 3, 5, and 6, and Amb t 5; pigweed Che a 2 and 5; and other weed allergens Par j 1, 2, and 3, and Par o 1);
grass (including major allergens Cyn d 1, 7, and 12; Dac g 1, 2, and 5; Hol I 1.01203; Lol p 1, 2, 3, 5, and 11; Mer a 1; Pha a 1; Poa p 1 and 5);
pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut);
dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blot 1, 2, 3, 4, 5, 6, 10, 11, 12, 13, 19, and 21; also allergens Eur m 2 from Euroglyphus maynei, Tyr p 13 from Tyrophagus putrescentiae, and allergens B1a g 1, 2, and 4; Per a 1, 3, and 7 from cockroach);
pets (including cats, dogs, rodents, and farm animals; major cat allergens include Fel d 1 through 8, cat IgA, BLa g 2, and cat albumin; major dog allergens include Can f 1 through 6, and dog albumin);
bee stings, including major allergens Api m 1 through 12; and
fungus, including allergens derived from, species of Aspergillus and Penicillium, as well as the species Alternaria alternata, Davidiella tassiana, and Trichophyton rubrum.

As will be appreciated by those skilled in the art, a patient can be tested to identify a tolerogenic antigen against which an unwanted immune response has developed, and a protein, peptide or the like can be developed based on that tolerogenic antigen and incorporated in a tolerogenic composition of the present disclosure.

Methods and materials used in the non-limiting experimental examples disclosed below are not to be read as limiting the embodiments disclosed herein.

### Example 1: Humanization of Mouse Antibody 10F7

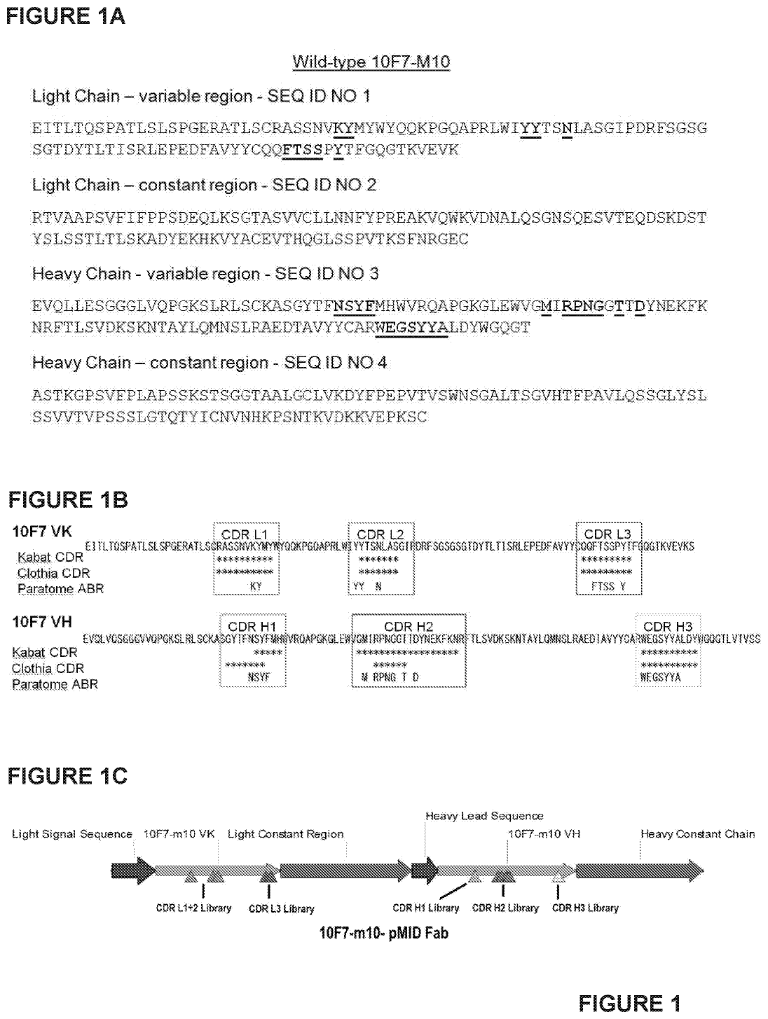

An anti-human glycophorin A (GPA) antibody (10F7) isolated from mouse (Bigbee et al., 1983) was selected as a candidate for erythrocyte targeting. 10F7 binds 100% of the human population, regardless of blood group type. Prior to affinity maturing 10F7 to improve binding properties, this antibody was first converted to an antibody fragment format and humanized. The heavy and light chains of mouse 10F7 antibody were humanized by the grafting of mouse complementary determining regions (CDRs) with human framework regions (FRs) to produce 10 candidate antibody sequences as Fab antibody fragments (“fAb”). Back mutations were introduced to preserve structure and affinity. These fAb antibodies were expressed, purified and evaluated for binding to human GPA by kinetic binding assays (ELISA, surface plasmon resonance) and for binding to human erythrocytes by flow cytometry. FIG. 1A depicts the light and heavy chains of the constant and variable regions of selected antibody sequences (termed “clone m10”), which were chosen for follow-on affinity maturation. The candidate GPA-binding residues mutated in the affinity maturation campaign of Example 2 are indicated by bold and underline.

### Example 2: 10F7 Affinity Maturation Campaigns

### Library Design and Construction

Computational structure-based modeling employing the Paratome algorithm was used to identify putative antigen binding regions (ABR) of 10F7—the amino acids in the CDRs believed to make physical contacts with human GPA. FIG. 1B depicts the complementarity-determining regions of the light and heavy chains of the variable region in accordance with the Kabat and Clothia schemes and as well as the locations of the Paratome ABRs. These ABRs were aligned with the CDRs and the identified ABR residues in the light and heavy chains were randomized in 5 separate libraries to affinity mature 10F7 (FIG. 1C). Phage display libraries for affinity maturation screening were generated by GeneArt/ThermoFisher Scientific with TRIM (trinucleotide mutagenesis)—assembly of pre-assembled trinucleotide blocks for customizable randomization of a library.

### Selection Process

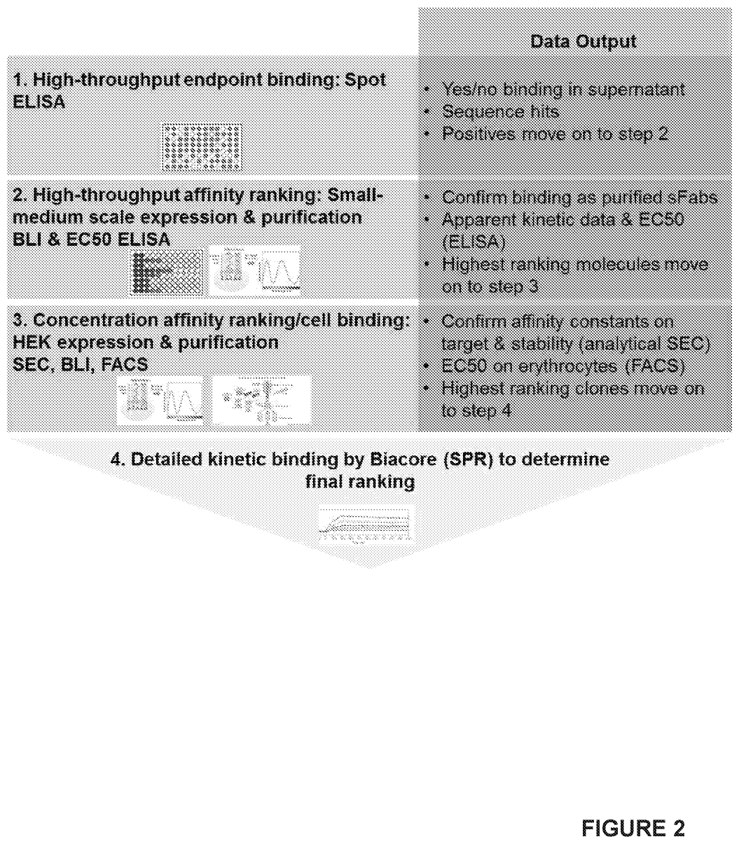

The resultant fAb library was displayed on M13 bacteriophage and subjected to three rounds of selections against recombinant biotinylated human GPA that was previously bound to streptavidin-coated magnetic beads. Stringency of the selection process was increased in each successive round of selection by reducing the GPA concentration and library-bead binding time, in order to promote selection of fAb antibody clones with enhanced affinity for GPA. After the third round of selection, the selected library output was screened by ELISA against human GPA to identify individual fAb clones and characterize their GPA binding properties. Clones that exhibited binding to GPA were sequenced, focusing mainly on the light chain variable region of the fAb. FIG. 2 summaries a schematic of the selection process.

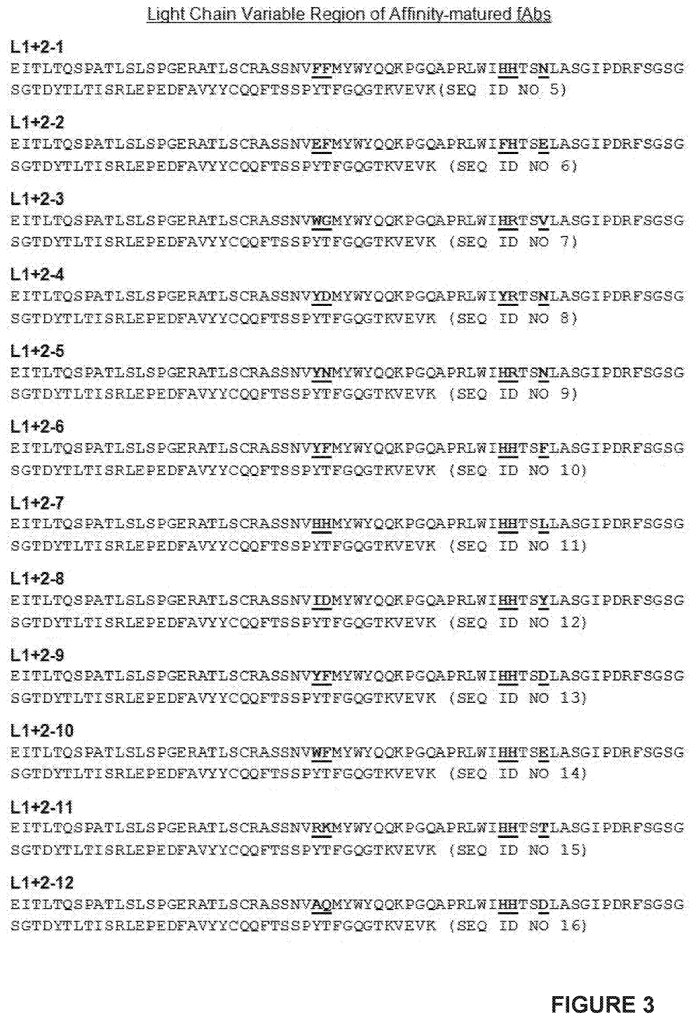

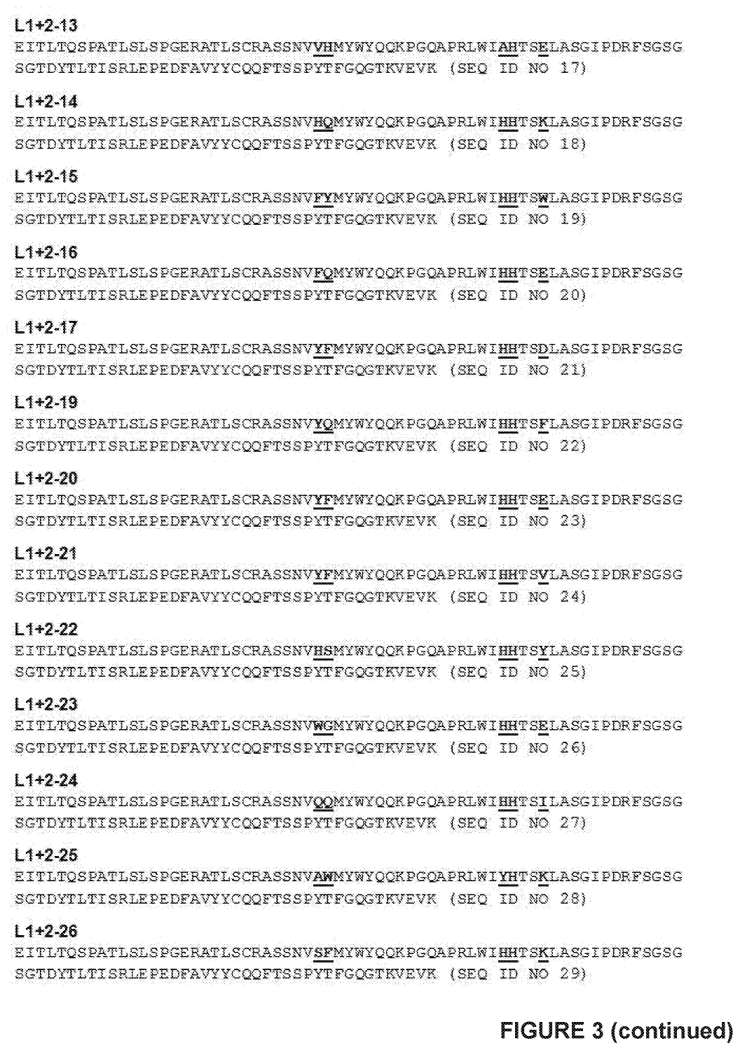

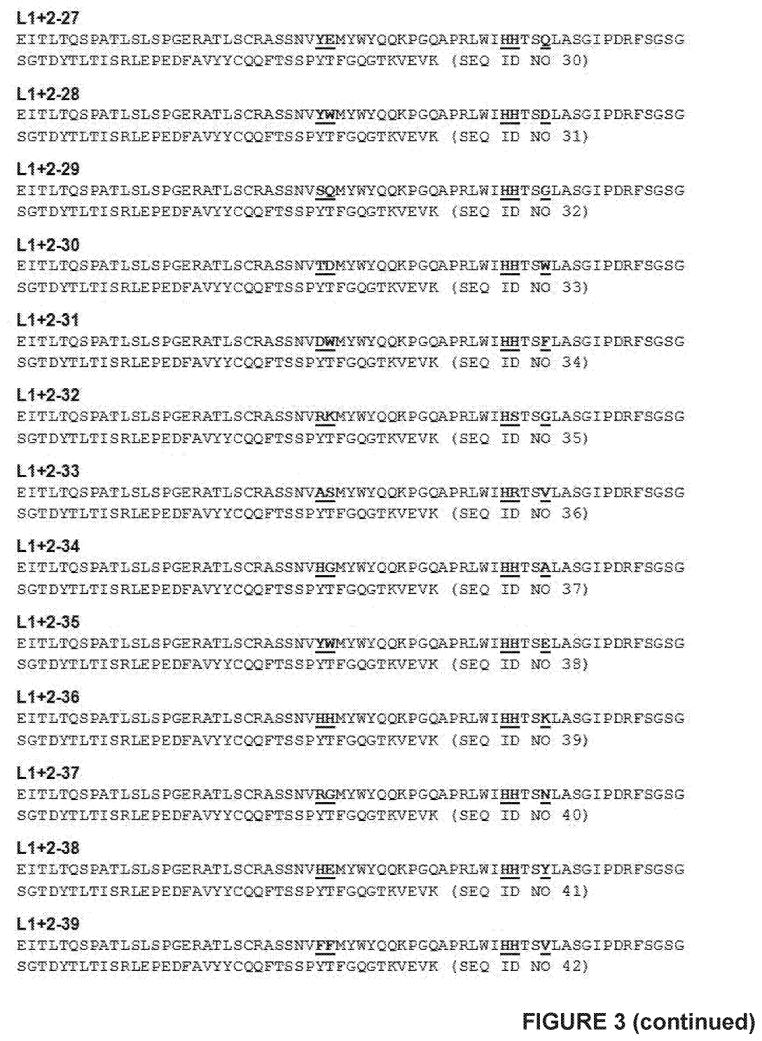

A total of 14 campaigns were completed, with the total number of hits identified from each library tallied in Table 5. An initial review of the libraries identified the L1+L2 CDR library (comprising mutations in the light chain CDR1 and CDR2 ABRs) as exhibiting enhanced affinity for human GPA. FIG. 3 depicts the sequences of light chain variable domains of affinity-matured fAbs (all other domains are the same as wild-type 10F7-M10), with the randomized ABR residues of CDR1 and CDR2 indicated by bold and underline.

TABLE 5
RESULTS OF 10F7-M10 AFFINITY
MATURATION CAMPAIGNS

| | | # Binding to hGPA | # Affinity Matured |
|---|---|---|---|
| CDR Libraries | # Hits | in Step 1 | Hits |
| L1 + L2 | 47 | 40 | 29 |
| L3 | 12 | 4 | 0 |
| H1 | 22 | 2 | 0 |
| H2 | 61 | 1 | 0 |
| H3 | 89 | 2 | 0 |
| New H1 | 1 | 1 | 0 |
| Total | 230 | 50 | 29 |

### Characterization and Ranking

Sequence-confirmed clones were recombinantly expressed in a soluble format (not fused to phage) in HEK cells and purified. All clones were assessed by analytical SEC to confirm that the Fabs were >97% monomer before characterization. The affinity of the top hits for human GPA was characterized by ELISA, biolayer interferometry, surface plasmon resonance, and flow cytometry assays on human erythrocytes. The starting (non-affinity matured 10F7-M10) was also characterized as a control. The results of this analysis are shown in Table 6, with clones L1+2-17 and L1+2-39 in particular exhibiting unexpectedly higher affinity towards human GPA.

TABLE 6
RESULTS OF 10F7-M10 AFFINITY MATURATION CAMPAIGNS

| | AA | | | | | | | | Analytical | |
|---|---|---|---|---|---|---|---|---|---|---|
| | Sequence | FACS | Octet (hGPA extract) | | | Biacore (hGPA extract) | | | SEC | Yield |
| | of | EC₅₀ | K_{D} app. | kₒₙ app | k_{off} app | K_{D} app. | kₒₙ app | k_{off} app | % | Conc |
| Clone ID | Library | (M) | (M) | (1/Ms) | (1/s) | (M) | (1/Ms) | (1/s) | Monomer | (mg/ml) |
| L1 + 2-39 | FF-HH-V | 2.7E−09 | 4.8E−09 | 2.5E+05 | 1.2E−03 | 4.82E−09 | 2.50E+05 | 1.21E−03 | 98.8 | 4.3 |
| L1 + 2-10 | WF-HH-E | 3.3E−09 | 6.4E−09 | 2.2E+05 | 1.4E−03 | 6.43E−09 | 2.22E+05 | 1.43E−03 | 99.7 | 6.1 |
| L1 + 2-1 | FF-HH-N | 3.3E−09 | 5.9E−09 | 3.1E+05 | 1.8E−03 | 5.95E−09 | 3.11E+05 | 1.85E−03 | 98.7 | 5.1 |
| L1 + 2-17 | YF-HH-D | 3.4E−09 | 6.0E−09 | 1.6E+05 | 9.3E−04 | 5.97E−09 | 1.56E+05 | 9.31E−04 | 98.7 | 4.9 |
| L1 + 2-15 | FY-HH-W | 3.4E−09 | 7.0E−09 | 2.2E+05 | 1.6E−03 | 7.00E−09 | 2.22E+05 | 1.55E−03 | 97.8 | 4.6 |
| L1 + 2-28 | YW-HH-D | 3.7E−09 | 3.5E−09 | 6.1E+05 | 2.1E−03 | 3.49E−09 | 6.14E+05 | 2.14E−03 | 98.8 | 6.1 |
| L1 + 2-21 | YF-HH-V | 3.7E−09 | 5.1E−09 | 2.6E+05 | 1.3E−03 | 5.12E−09 | 2.57E+05 | 1.32E−03 | 98.6 | 4.5 |
| L1 + 2-35 | YW-HH-E | 4.6E−09 | 1.7E−08 | 8.1E+04 | 1.4E−03 | 1.67E−08 | 8.13E+04 | 1.36E−03 | 99.1 | 5.2 |
| L1 + 2-16 * | FQ-HH-E | 4.6E−09 | 6.18E−09 | 6.1E+05 | 3.8E−03 | 6.18E−09 | 6.08E+05 | 3.76E−03 | 98.5 | 5.7 |
| L1 + 2-19 * | YQ-HH-F | 4.6E−09 | 6.83E−09 | 4.8E+05 | 3.3E−03 | 6.83E−09 | 4.77E+05 | 3.26E−03 | 99.2 | 6.9 |
| L1 + 2-14 | HQ-HH-K | 6.4E−09 | 3.3E−08 | 1.7E+04 | 5.7E−04 | 3.28E−08 | 1.73E+04 | 5.68E−04 | 97.5 | 5.0 |
| L1 + 2-7 * | HH-HH-L | 6.9E−09 | 2.3E−08 | 1.6E+05 | 3.7E−03 | 2.32E−08 | 1.60E+05 | 3.71E−03 | 98.3 | 5.4 |
| L1 + 2-5 * | YN-HR-N | 8.8E−09 | 1.0E−08 | 7.9E+05 | 8.1E−03 | 1.02E−08 | 7.95E+05 | 8.08E−03 | 99 | 6.9 |
| L1 + 2-8 | ID-HH-Y | 1.4E−08 | 1.1E−08 | 2.0E+05 | 2.2E−03 | 1.10E−08 | 1.98E+05 | 2.18E−03 | 99.1 | 6.1 |
| L1 + 2-24 | QQ-HH-I | 2.3E−08 | 2.1E−08 | 3.8E+05 | 8.0E−03 | 2.10E−08 | 3.82E+05 | 8.02E−03 | 98.6 | 6.5 |
| 10F7-M10 | KY-YY-N | 7.3E−08 | 1.4E−07 | 4.7E+04 | 6.7E−03 | 1.41E−07 | 4.73E+04 | 6.67E−03 | 98.8 | 5.2 |
| L1 + 2-25 | AW-YH-K | 1.0E−07 | | | | | | | 98.1 | 6.6 |

* Biphasic off-rate to fit data

### Example 3: Next Generation Sequencing-Based Affinity Maturation

### Brief Overview

The present example relates to methods, and resulting antibodies, discovery of candidate antibodies, or fragments thereof (e.g., a Fab) that bound human erythrocytes irrespective of blood type group antigens. Another goal was to achieve species cross-reactivity, specifically such that a successful candidate is also cross reactive to the cynomolgus monkey (cyno) homologue protein. An affinity maturation campaign was developed to identify high affinity clones that bound both the human and cyno target protein backbone, independent of post translational modifications. Phage-displayed combinatorial scfv libraries were designed to randomize each of the CDRs of the parental Fab individually. Each CDR library was then panned against several variants of the human and cyno target protein to maintain cross-reactivity and to ensure that differences in post-translational modifications would not impact binding. A Next Generation Sequencing (NGS) based primary screen was developed to identify affinity matured hits. Data from the NGS screen were compared to those from a scfv-phage ELISA screen, and it was determined that while both screens identified the top hits of each campaign, the NGS analysis also provided a broader insight into the success of the selections. By profiling the outputs of each round of phage panning via NGS, incremental enrichment of sequence motifs was more easily detectable, which provided a better prediction for affinity improvements. The data also indicate that the NGS approach was more efficient at identifying hits than traditional ELISA approaches, with reduced labor and costs. By implementing this streamlined methodology we were able to affinity mature a Fab 6- and 16-fold to human and cyno red blood cells, respectively, to achieve high-affinity binding to all blood types tested.

### Discovery and Humanization

During a hybridoma campaign in which mice were immunized with cyno RBCs and boosted with cyno glycophorin A, an IgG antibody was discovered, termed 3-103. It should be noted that the methods disclosed herein are applicable to various types of antibodies, including IgA, IgD, IgE, IgM, or subtypes of any such antibody class. In several embodiments the methods are particularly suited to IgG2 antibodies, such as IgG2a, by way of example only. The heavy and light chains of the 3-103 antibody were humanized by grafting of mouse CDRs with the human framework regions to produce candidate constructs. Back mutations were introduced to preserve structure, affinity, and improved E. coli expression of the antibodies as soluble Fabs. These constructs were expressed as soluble Fabs, purified and evaluated by stability assays and kinetic assays with human and cyno GPA and red blood cells. Clone M1 (“3-103M1”) was chosen for further affinity maturation.

### Library Design and Construction

Computer modeling was used to identify predicted residues in the CDRs. Libraries were rationally designed to targeted and randomize the predicted light chain CDR1 and CDR2 and heavy chain CDR1 and CDR2 to generate 5 libraries for affinity maturation screening. Genes were synthesized by GeneArt and cloned into the pBs-3-103M1-CMVd1 vector for the creation of scfv-phage combinatorial libraries.

### Selection Process

The library was phage displayed and subjected to three rounds of selections against biotinylated recombinant human GPA, recombinant cyno GPA, and human GPA extracted from red blood cells (extract). Scfv-phage bound to biotinylated antigen was captured with streptavidin-coated magnetic beads. Nine different selection conditions were run for each library to ensure that each would be selected with the 3 formats of GPA and that each library would see a combination of both species of antigen. After each round of selections, the antigen type or concentration was altered, and the binding incubation time of library and antigen was decreased. After the third round of selection, the library outputs were sent for AmpliconEZ NGS analysis by GeneWiz and the phage outputs were screened by scfv-phage spot ELISA in parallel. Positive ELISA hits were confirmed by Sanger sequencing of the appropriate variable regions.

### NGS Analysis

NGS-based amplicon sequencing was performed by GeneWiz AmpliconEZ service. Analysis of amino acid unique sequence frequency was delivered. Fold enrichment of each sequence was calculated by dividing the frequency of each sequence by the frequency observed in the preceding rounds of phage display or selections. Sum Fold Enrichment scores were also calculated to take into account the enrichment of sequences across all rounds of selections. Sums of enrichment were calculated by adding the fold enrichments from at least 2 rounds of selections. Clones were typically ranked by the sum fold enrichment of Round 3 Output vs Round 2 Output+Round 3 Output vs Round 1 Output+Round 3. Sequences were selected as hits for further characterization if they had a sum fold enrichment score above the median score of all clones. For example, if a list of top hits from one comparison condition had a median Sum Fold Enrichment value of 32, generally the clones with a sum fold score >32 were selected for recombinant expression. The sequences were further evaluated for potential sequence liabilities (glycosylation site, Asn deamidation and Asp isomerization) and sequences with any potential liability were excluded.

### Characterization and Ranking

Top sequences identified by NGS and ELISA were expressed as soluble Fabs in high throughput format in HEK cells. The Fab concentration in supernatant was quantified by bio-layer interferometry (BLI/Octet® analysis) and clones in supernatant were tested for affinity to human red blood cells by flow cytometry. Top ranking clones were purified with CH1 resin and characterized by flow cytometry for binding to human and cyno erythrocytes.

### Library Panning

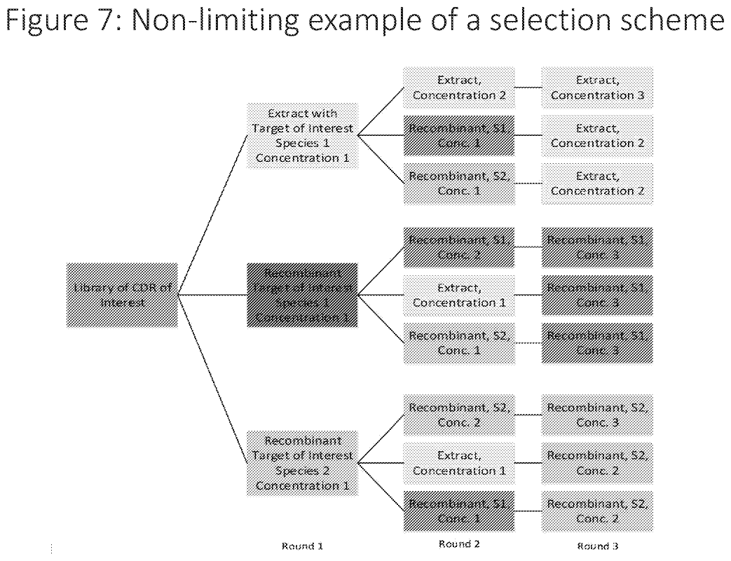

FIG. 7 shows a non-limiting schematic of a library panning protocol. A given CDR library is, according to some embodiments, subjected to three (or more) rounds of selection. Round 1 is conduct with three concentrations of a target antigen. Any target of a candidate antibody can be used. By way of non-limiting example, selection was performed using human glycophorin A or cyno glycophorin A as the targets, though other targets or species can be used, according to several embodiments. The output titers of a given round are used to select staring antigen concentration for the subsequent round. In this experiment, conditions with the lowest antigen concentration that produce Round 1 output titers of 1×10⁶-1×10⁸ CFU/mL were chosen. Antigen concentrations were reduced by 10× for each subsequent round (unless the antigen was changed). In additional embodiments, different reductions can be used, e.g., 2×, 5×, 8×, 12×, 15×, 20×, 50×, etc. When alternating antigens (e.g., human to cyno or vice versa), the concentration of the antigen was held constant between rounds. Binding incubations of library to biotin-antigen complexes were decreased for each successive round of selection, but streptavidin bead capture time and washes were held constant.

### Results and Discussion

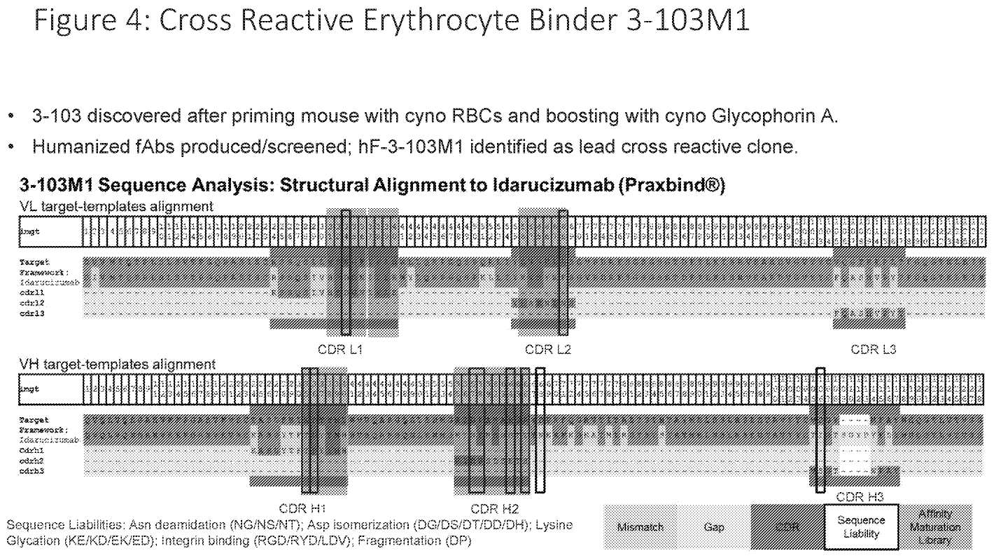

As discussed above, there was undertaken an affinity maturation campaign based on various CDRs of an identified antibody that binds to both human and cyno glycophorin A. FIG. 4 shows a schematic representation of a candidate antibody that was capable of binding both species of glycophorin A, termed 3-103. Fabs were generated and humanized and screened, with hF-3-103M1 being identified as a highly cross-reactive clone. FIG. 4 depicts a sequence alignment of the VL and VH chains of 3-103M1 and identifies several CDR regions that were targeted for affinity maturation.

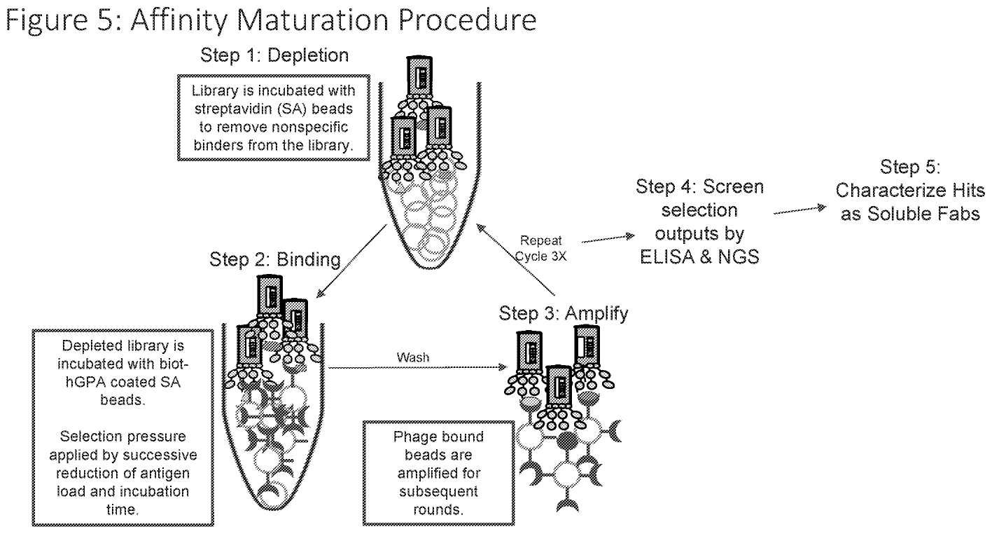

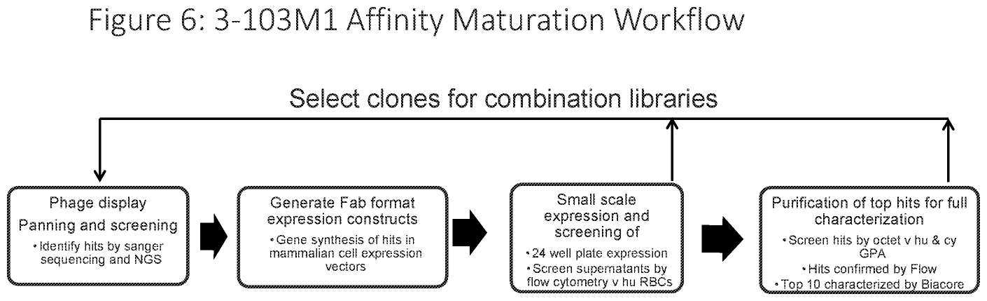

FIG. 5 depicts a schematic of the affinity maturation protocol. A phage library of Fabs is first depleted through incubation of the library with streptavidin (SA) beads in order to remove non-specific binders from the library. The depleted library is then incubated with biotin-human glycophorin A coated SA beads. A selection pressure to identify antibody fragment with higher affinity for glycophorin A is applied through the successive reduction of antigen concentration applied to the phage library and a concurrent reduction in incubation time. This helps to identify the Fabs with the higher affinity to the target. Those phage that are bound to glycophorin A beads are amplified for subsequent rounds of screening. In this schematic, three rounds of screening are performed, though in several embodiments, greater numbers of screening rounds can be performed. After three rounds, the output candidates Fabs are evaluated by both ELISA-based and NGS-based methods. Finally, the hits are characterized as soluble Fabs. FIG. 6 depicts a schematic of the workflow pathway used according to several embodiments to affinity mature an antibody, or antibody fragment.

As discussed above in the Materials and Methods, FIG. 7 depicts a non-limiting example of a selection scheme used to identify high affinity Fabs. Shown here is a selection process for the CDRL1B library. As discussed above, Round 1 was conducted with three concentrations of the human glycophorin A target antigen. The output titers of a given round are used to select staring antigen concentration for the subsequent round. By this approach, the output of each round should selectively cull from the candidate population those antibodies or antibody fragments with less affinity for the target. Those candidates that do show elevated affinity can subsequently be characterized.

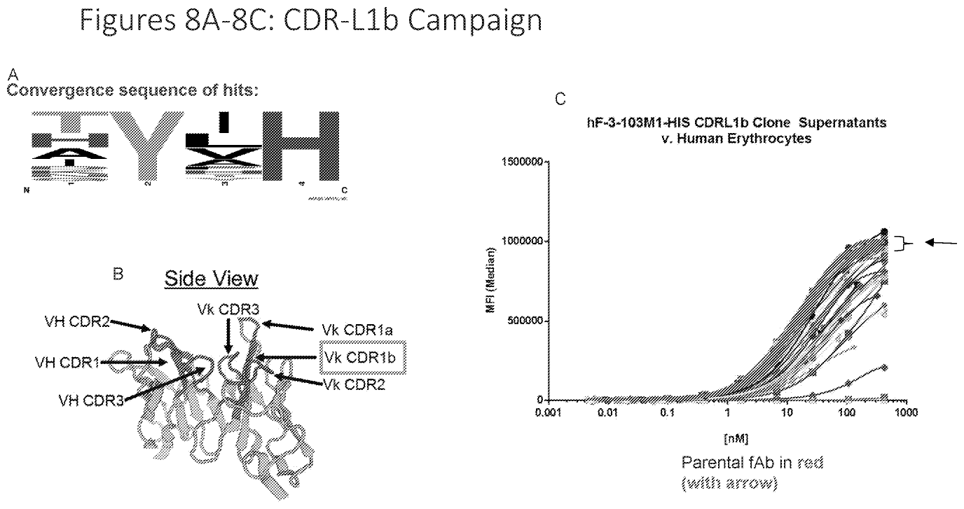

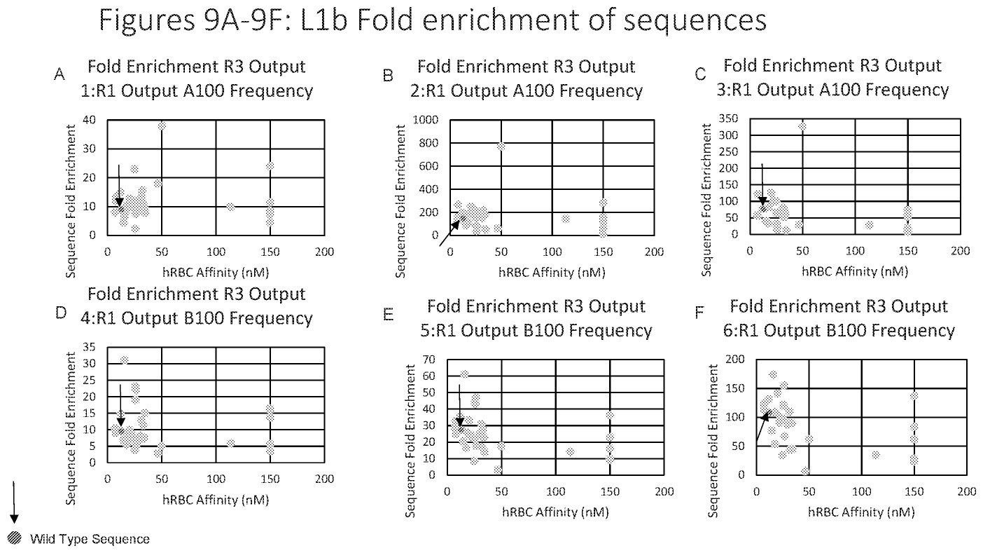

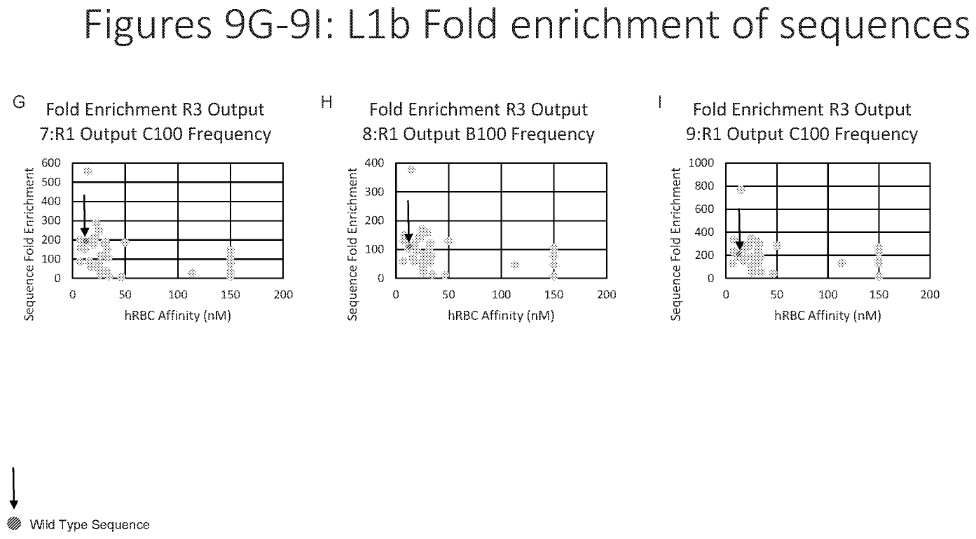

FIGS. 8A-8C show data related to the output of the selection process being applied to the CDRL1B affinity maturation campaign. FIG. 8A shows the weighted consensus sequence schematic. The selection process demonstrated that the Library converged onto sequences that resemble wild-type (non-matured) sequence (TYLH). It also appears that amino acid residues 2 and 4 appear substantially fixed as —Y—H based on this selection campaign. FIG. 8B shows a schematic ribbon structure of an antibody with the CDR1B position identified with a box. Subsequent characterization of the candidates showed that more than half of hits did not express as soluble fAbs. Moreover, as shown with the binding assay results in FIG. 8C, those that did express, did not have significantly improved affinity to either human or cyno RBCs over parental fAb (generally identified with an arrow). There are many curves that produce a reduced MFI as compared to the parental Fab, indicative of a lesser affinity to human erythrocytes (that express the target Glycophorin A). Additional data is shown in FIGS. 9A-9I which graphically depict the degree of sequence fold enrichment vs. affinity for human red blood cells for each of the output parameters of the selection scheme shown in FIG. 7. Sum Fold Enrichment scores were also calculated to take into account the enrichment of sequences across all rounds of selections. Sums of enrichment were calculated by adding the fold enrichments from at least 2 rounds of selections. Clones were typically ranked by the sum fold enrichment of Round 3 Output vs Round 2 Output+Round 3 Output vs Round 1 Output+Round 3 Output. Sequences were selected as hits for further characterization if they had a sum fold enrichment score above the median score of all clones. By way of example, FIG. 9A shows data related to affinity resulting from Output 1 of Round 3 in FIG. 7 (upper right box—hGPA extract, 20 fmol) as compared to the affinity in Round 1 Output A (hGPA extract 200 pmol). Likewise, FIG. 9B shows data for Output 2 of Round 3 in FIG. 7 as compared to Round 1 Output A. In line with the binding data shown in FIG. 8C, these scatter plots show that the majority of the sequences that were enriched during this campaign, exhibit similar affinities for human erythrocytes as compared to the wild type parental Fab (shown by an arrow). These data additionally highlight that the parental Fab sequence was also enriched during selections. This reinforces the initial hypothesis that this domain of CDRL1 may need to be conserved to maintain its potential role in a structural or other characteristic of the Fab that supports binding to antigen.

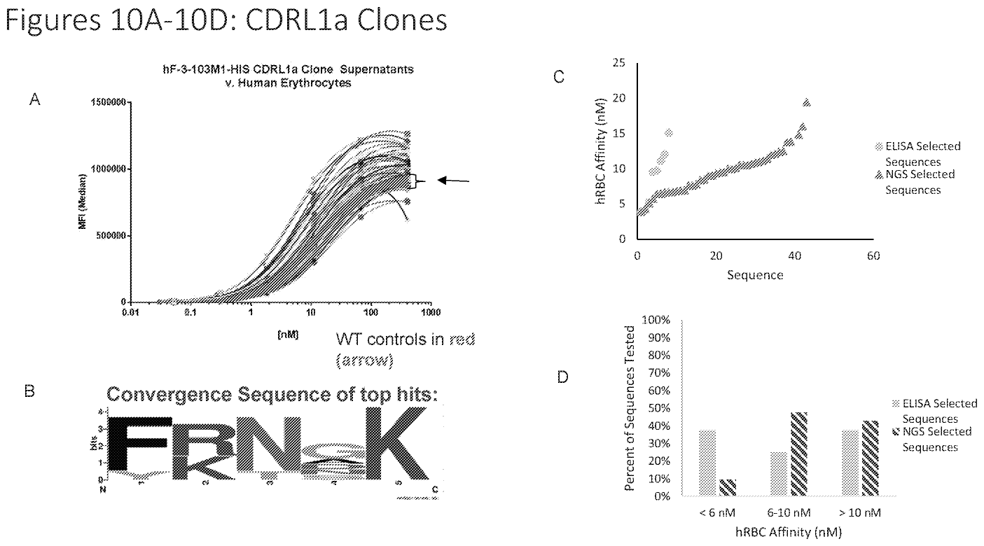

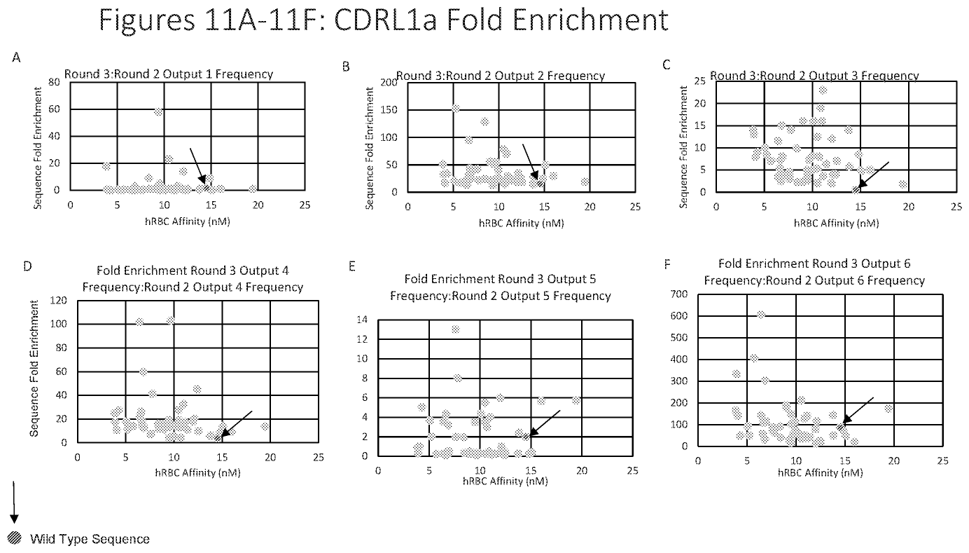

FIGS. 10A-10D show data summarizing the characteristics of clones run through the selection process of FIG. 7, but utilizing the CDRL1a library. FIG. 10A shows that numerous CDRL1a clones generated antibodies that have enhanced affinity for human erythrocytes, as compared to the parent Fab (indicated generally by the arrow). FIG. 10B shows a schematic of the convergence sequence of selected hits from the library. FIG. 10C shows data comparing the affinity of antibody fragments identified by an ELISA-based method (filled circle) versus the NGS-based methods described herein (and used in this Example). These data show that a larger number of high affinity antibodies are identified using NGS. The larger number is due, at least in part, to the greater throughput that NGS-based methodology offers. Nearly 40 sequences were screened, as compared to 6-8 using ELISA methodology. Moreover, many of these antibodies were in the low nM affinity range, which is higher affinity than many of the ELISA-identified Fabs. FIG. 10D shows the data from FIG. 10C as the percent of clones identified from the NGS or ELISA primary screens broken down by affinity ranges for human erythrocytes (note—the sum of the type of Fab across all affinity ranges equals 100%). As indicated about 10% of the sequences identified using NGS have <6 nM affinity for human erythrocytes, as compared to about 38% using ELISA. However, at 6-10 nM or >10 nM affinity, the percentage of sequences identified using NGS methods was greater than that expressed using ELISA methods. The ELISA screen identified 5× fewer clones than the NGS screen did, but a higher percentage of the clones identified in this library were high affinity binders with <6 nM EC50 to human erythrocytes. It is important to note that these same high affinity clones were also identified by the NGS screen. FIGS. 11A-11F show data related to the fold enrichment of CDRL1a candidates. As described above, each panel is related to a specific Output comparison from the selection scheme to determine a fold enrichment. FIG. 11A shows data related to the Round 3 Output 1 affinity as compared to Round 2 Output 1. In contrast to the data of FIG. 8, the data of FIG. 11A-11F indicate that there is an enhanced affinity for the target that results from the selection/maturation process and that the parental sequence was outcompeted by the affinity matured clones. Notably, many of the data points are shifted to affinity values in the nM range (e.g., higher affinity for the target) as compared to the parent Fab (indicated with an arrow). Thus, the maturation campaign and selection process using NGS methods resulted in identification of Fabs with enhanced target affinity. Given the more rapid nature of NGS-based methods, and the higher affinity Fabs identified, in several embodiments, use of an NGS-based approach can provide for a more robust screening of candidate antibodies, at a reduced cost.

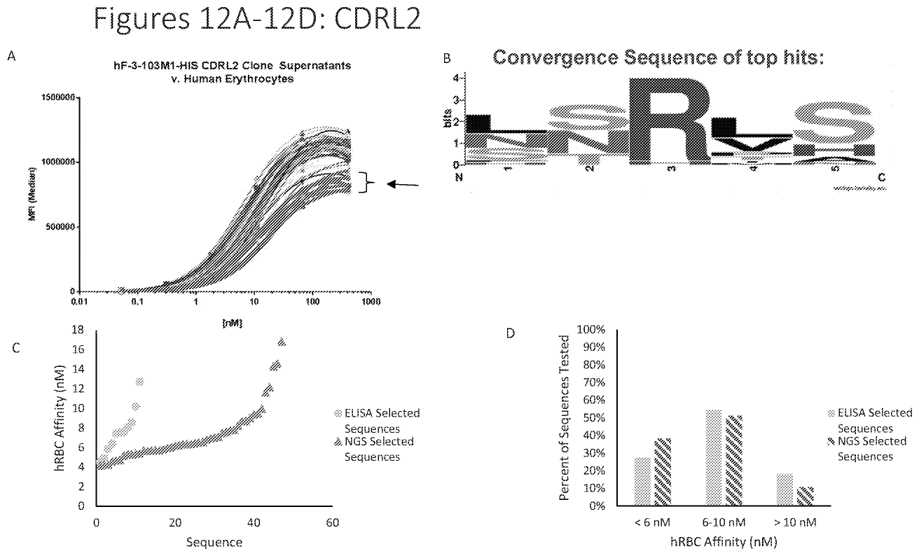

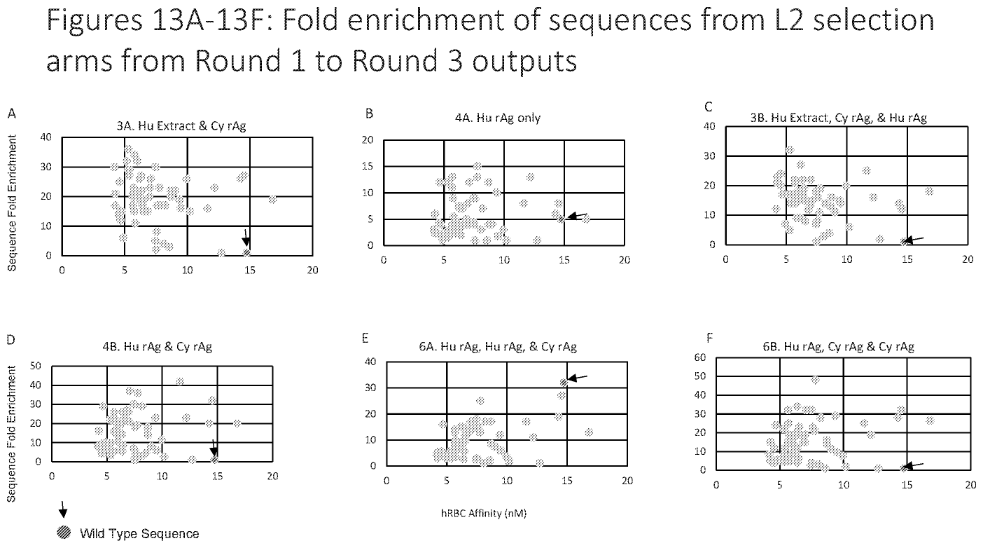

FIGS. 12A-12D show data summarizing the characteristics of clones run through the selection process of FIG. 7, but utilizing the CDRL2 library. FIG. 12A shows that numerous CDRL2 clones generated antibodies that have enhanced affinity for human erythrocytes, as compared to the parent Fab (indicated generally by the arrow). FIG. 12B shows a schematic of the convergence sequence of selected hits from the library. FIG. 12C shows data comparing the affinity of antibody fragments identified by an ELISA-based method (filled circle) versus the NGS-based methods described herein (and used in this Example). As above, these data show that a larger number of high affinity antibodies are identified using NGS and that NGS-methods have a greater throughput, with over 45 candidate clones resulting. FIG. 12D shows the data from FIG. 12C as a percent of clones identified from the NGS and ELISA primary screens broken down by affinity for human erythrocytes. As indicated, nearly 40% of the sequences identified using NGS have <6 nM affinity, as compared to about 25% of the sequences identified using ELISA. Those with 6-10 nM and >10 nM affinity were similar between ELISA and NGS methods. FIGS. 13A-13F show data related to the fold enrichment of CDRL2 candidates. Here again, many of the candidates exhibit notable enrichment which manifests as enhanced affinity as compared to the wild-type (arrow).

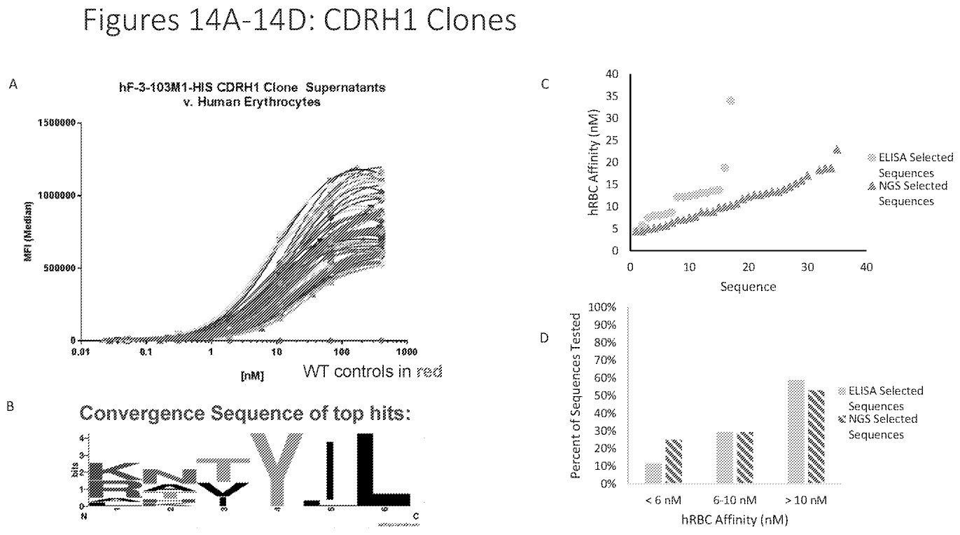

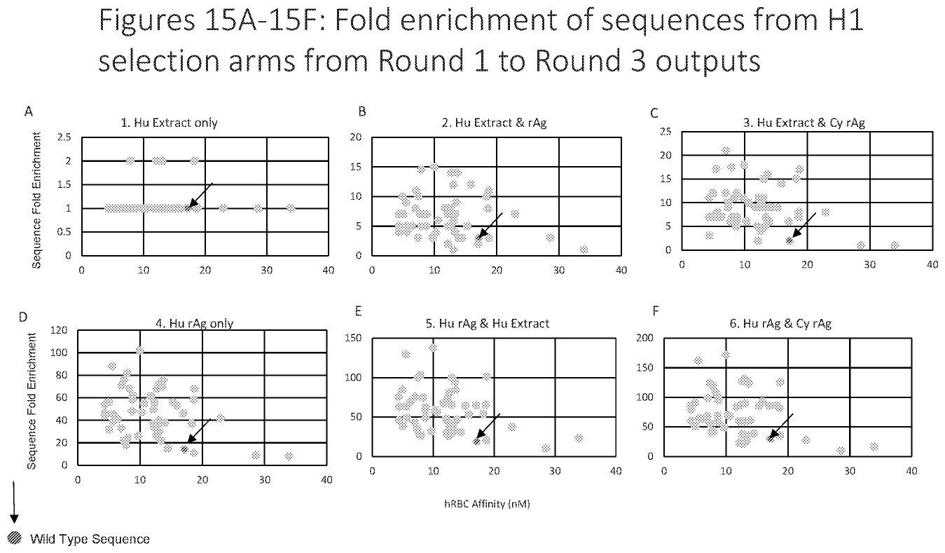

FIGS. 14A-14D show data summarizing the characteristics of clones run through the selection process of FIG. 7, but utilizing the CDRH1 library. FIG. 14A shows a wide distribution of affinity for human erythrocytes from the CDRH1 clones, as compared to the parent Fab. FIG. 14B shows a schematic of the convergence sequence of selected hits from the library. FIG. 14C shows data comparing the affinity of antibody fragments identified by an ELISA-based method (filled circle) versus the NGS-based methods (triangle) described herein (and used in this Example). As above, these data show that a larger number of high affinity antibodies are identified using NGS and that NGS-methods have a greater throughput, with over 30 candidate clones resulting. FIG. 14D shows the data from FIG. 14C as the percent of clones identified from the NGS and ELISA primary screens broken down by affinity for human erythrocytes. As indicated, the number of sequences identified using NGS having <6 nM was about two times more than those identified using ELISA. Expression was similar between the two methods for 6-10 nM and >10 nM affinities. FIGS. 15A-15F show data related to the fold enrichment of CDRH1 candidates. Here again, many of the candidates exhibit enhanced affinity as compared to the wild-type Fab (arrow). These data support the concept that, according to several embodiments, NGS-based methods are highly efficient at predicting the success of identifying antibodies, or antibody fragments, that exhibit enhanced affinities for a target.

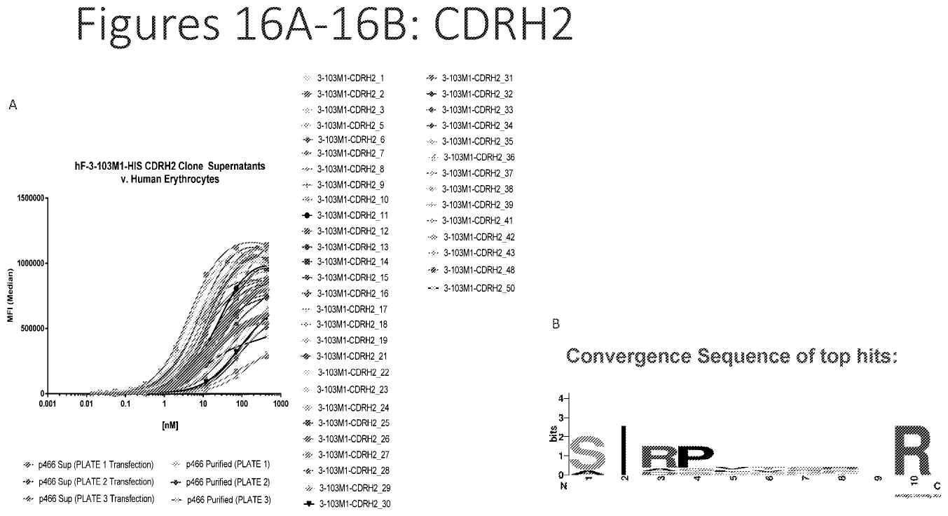

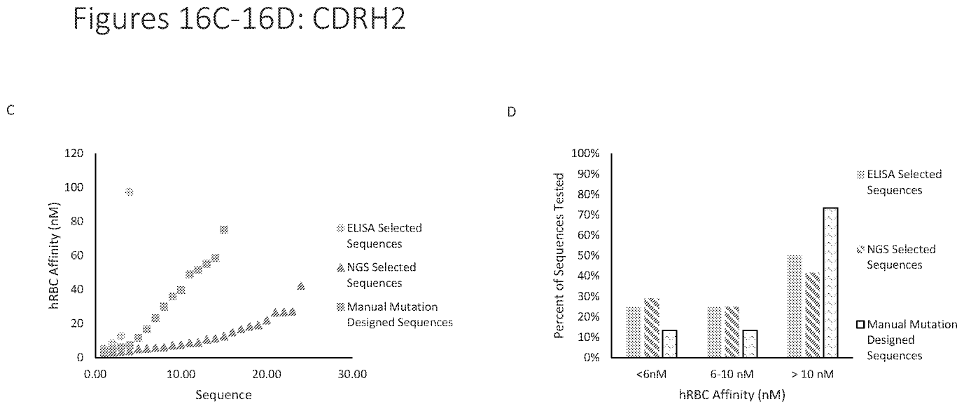

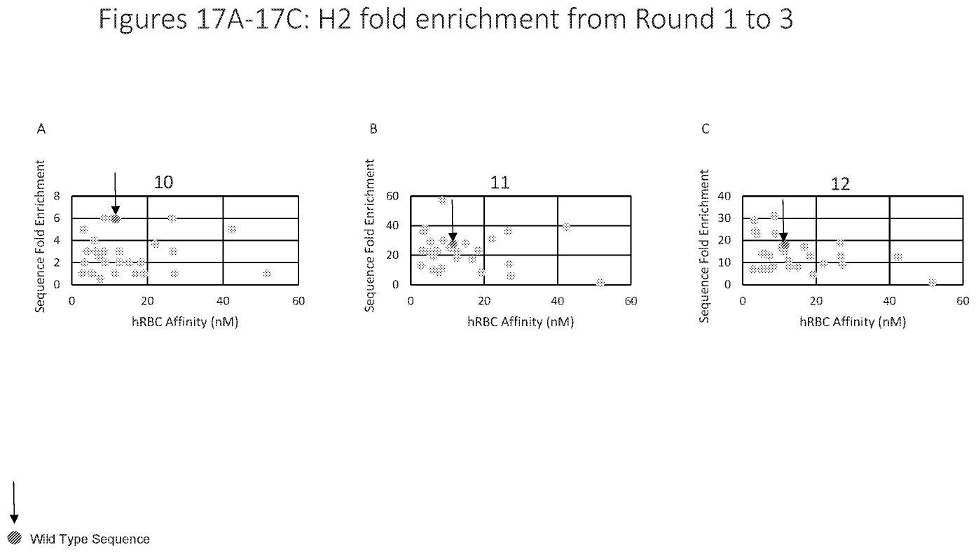

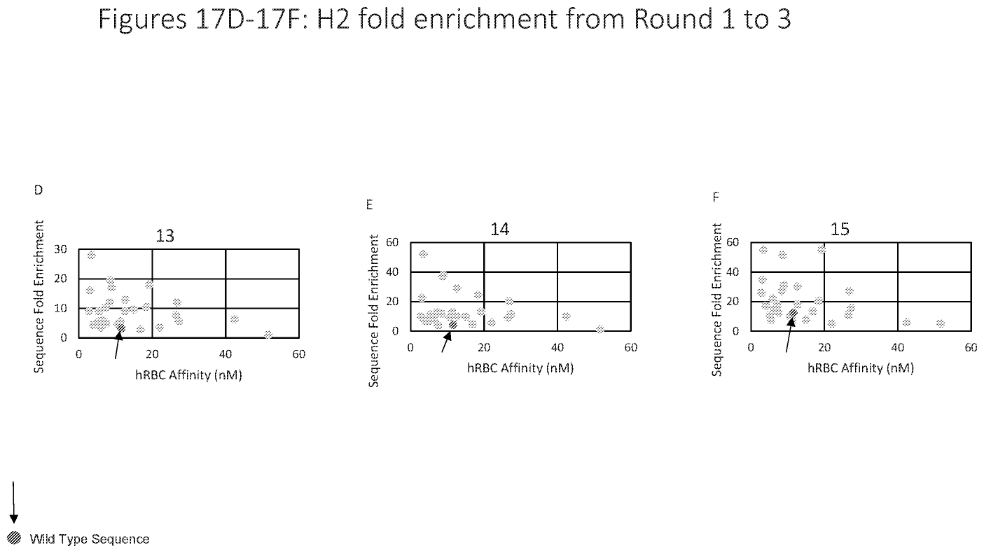

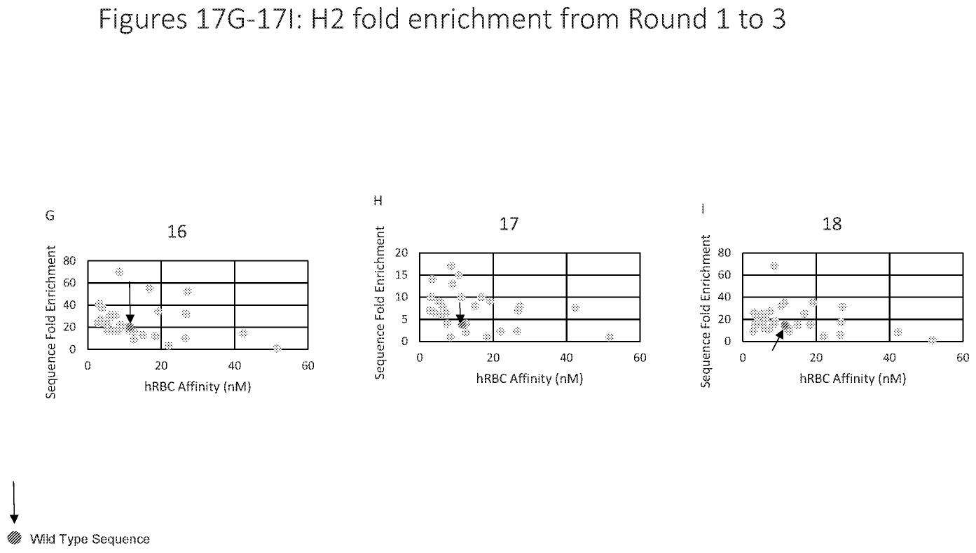

FIGS. 16A-16D show data summarizing the characteristics of clones run through the selection process of FIG. 7, but utilizing the CDRH2 library. FIG. 16A shows a wide distribution of affinity for human erythrocytes from the CDRH2 clones, as compared to the parent Fab. FIG. 16B shows a schematic of the convergence sequence of selected hits from the library. FIG. 16C shows data comparing the affinity of antibody fragments identified by an ELISA-based method (filled circle) versus the NGS-based methods (triangle) described herein (and used in this Example) and as compared to manually mutation design sequences (square). As above, these data show that a larger number of high affinity antibodies are identified using NGS and that NGS-methods have a greater throughput as compared to ELISA or manual methods. FIG. 16D shows the data from FIG. 16C as percent of clones identified from the NGS and ELISA primary screens broken down by affinity to human erythrocytes. As indicated, the number of sequences identified using NGS having <6 nM affinity is greater than those identified using ELISA and over two times more than manual mutations. Expression was similar between the NGS and ELISA methods for 6-10 nM affinities. While there were some variations in percent of high affinity clones with <6 nm EC50 to human erythrocytes, there was no substantial difference in the attrition of clones between the two screening formats, which was the case for all library outputs analyzed in this experiment. At the lowest affinity range, manual approaches outpaces both and ELISA and NGS methods. However, as seen with the other CDR libraries, the greater throughput and elevated percent identification of high affinity clones suggests that NGS is a highly efficient methodology. FIGS. 17A-17I show data related to the fold enrichment of CDRH2 candidates. As with the H1 library, many of the candidates exhibit enhanced affinity as compared to the wild-type Fab (arrow). These data support the concept that, according to several embodiments, NGS-based methods are highly efficient at predicting the success of identifying antibodies, or antibody fragments, that exhibit enhanced affinities for a target.

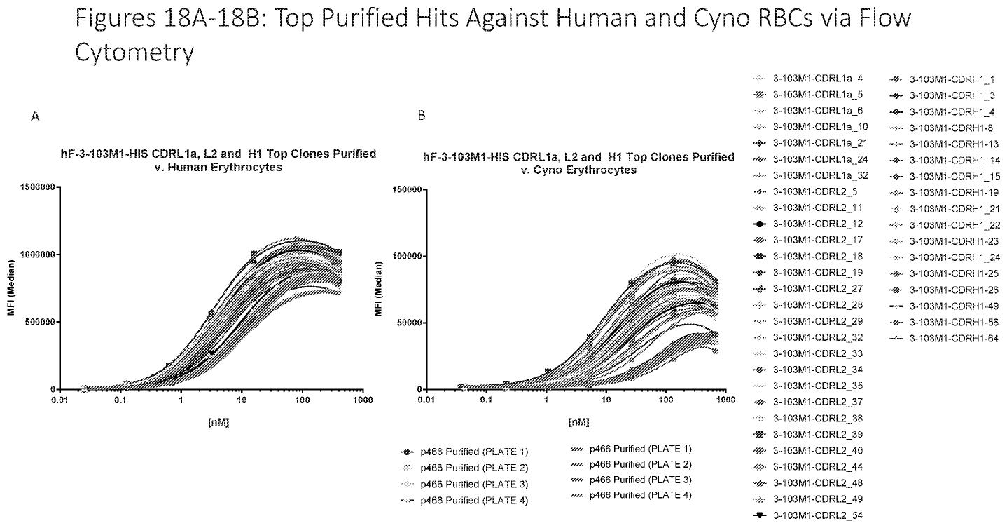

FIGS. 18A-18B show data related to the ability of selected clones with enhanced target affinity to bind both human (18A) and cyno (18B) erythrocytes. As can be seen, numerous clones show binding to both species of erythrocyte, and the majority of the clones exhibit enhanced affinity for the target as compared to the wild type parent Fab.

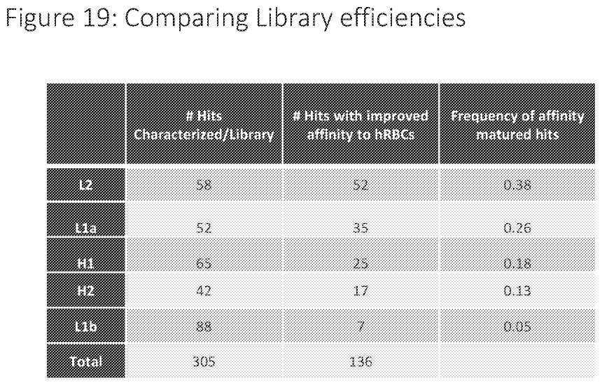

FIG. 19 shows a comparison of the efficiencies of the each of the libraries testing in this example. With a total of 305 hits characterized overall and 136 total hits showing enhanced efficacy to erythrocyte binding, the L2 library, with 52 hits showing improved affinity, exhibited a frequency of 0.36. Thus, L2 was the most successful library at producing the top affinity matured hits to both human and cyno erythrocytes.

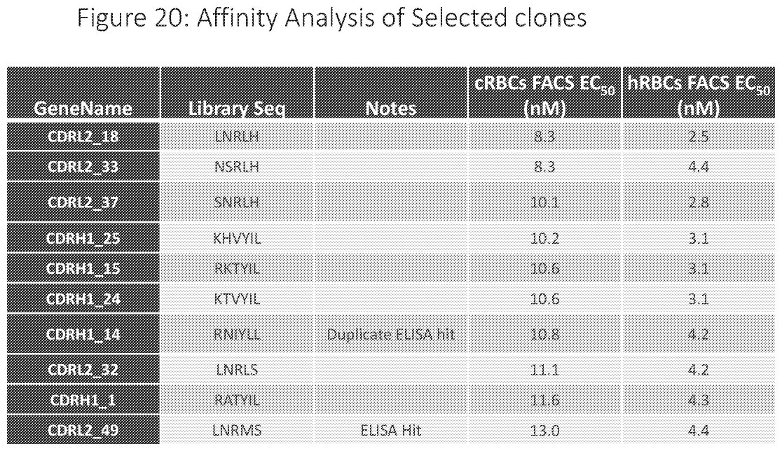

FIG. 20 summarizes data related to the affinity as measured by FACS for both cyno and human erythrocytes. The target of the affinity maturation campaign was to have single digit nM affinity (1-9 nM) to human erythrocytes. As shown in the far right column, single digit nM affinity (2.5-4.4 nM) was achieved for at least the ten identified clones. The second column from the right also shows an enhanced affinity for cyno erythrocytes.

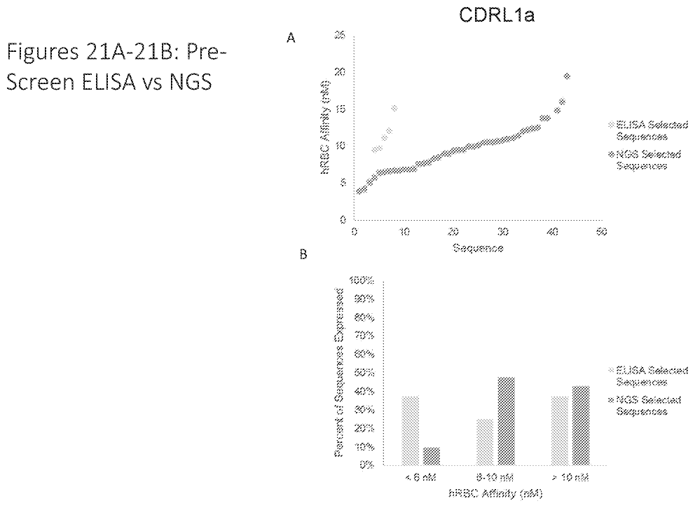

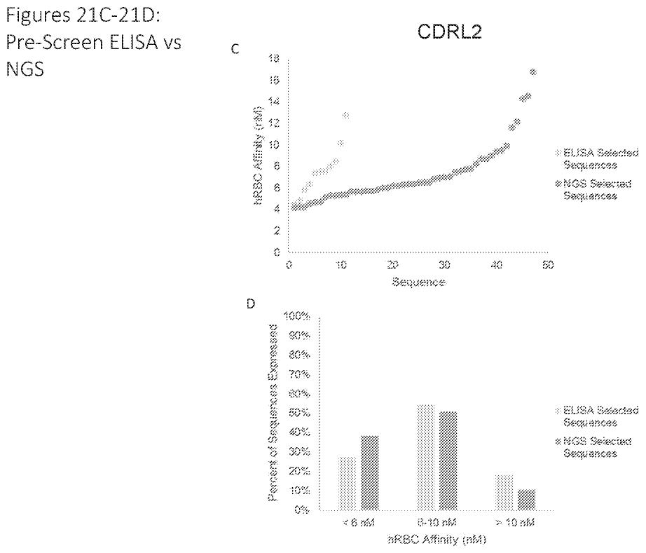

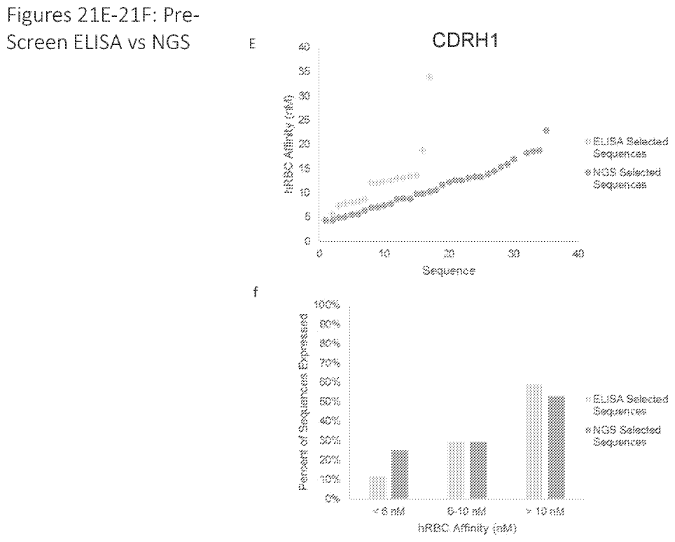

FIGS. 21A-21F show summarized data related the affinities (and quantities) of sequences evaluated by ELISA vs. NGS. Summarizing the data discussed above, FIGS. 21A, 21C, and 21E show that one advantage of screening by NGS is the ability to screen a wider panel of clones that covers a broad range of affinities. Advantageously, this reduces costs and time invested in a maturation campaign. Also, the clones exhibiting the highest affinity were by both ELISA and NGS screen, which can allow for a two-method quality control exercise. Further while there are some variations in the percent of identified high clones (e.g., <6 nM affinity for human erythrocytes), there is not a significant difference in attrition of candidates between the two screening formats. Thus, this example demonstrates certain advantages of screening affinity matured candidates using and NGS-based methodology.

### Example 3A: Further Data Regarding Next Generation Sequencing-Based Affinity Maturation

Further refining Example 3, above, experiments were performed to identify a fragment antibody (fAb) specific to an erythrocyte membrane protein, glycophorin A. Fab constructs were affinity matured by 6- & 16-fold to human and cynomolgus monkey (cyno) erythrocytes, respectively (also referred to as hRBCs or cRBCs, respectively). Cross reactive, high-affinity binding to erythrocytes irrespective of blood type group antigens was achieved. A Next Generation Sequencing (NGS) based primary screen was developed to identify affinity matured hits. The NGS method proved to be not only more efficient at identifying hits than traditional ELISA approaches, but was also less labor intensive and more cost efficient. Summaries of both ELISA and NGS methods are provided below in Table 6.

TABLE 6
Summary of ELISA and NGS Screening Methods

| | Spot ELISA | NGS |
|---|---|---|
| Description | Qualitative ELISA screen for | ssDNA extracted from each round of |
| | binding to selection | selections and library regions |
| | antigens. | amplified for NGS. |
| | Clones screened in | Sum Fold Enrichment (SFE) scores |
| | supernatant as scfv-phage | were calculated by dividing the |
| | fusions. | sequence frequency from Round 3 |
| | | by that of Round 1 and adding this |
| | | fold enrichment to that of |
| | | Round 3:Round 2. |
| Pros | Quick - 2 day screen + 1 | 50,000+ reads allow for greater |
| | week for sequencing. | library coverage |
| | Cheap reagents. | Can filter out mutated clones. |
| | Functional screen. | Allows for quantification of library |
| | | convergence and prediction of |
| | | selection success. |
| | | Cheaper than sequencing individual |
| | | ELISA hits. |
| Cons | No normalization for | No expression or functional data. |
| | concentration, implicit bias | 2 week lead time. |
| | for high expressing clones. | May require CRO. |
| | Limited library coverage. | |
| | High false positive rate due | |
| | to mutated/bald phage. | |

### Methods

As discussed above in Example 3, combinatorial scFv-phage libraries were designed to randomize each CDR individually. Selections of candidate antibodies were performed using both human and cyno target antigen to maintain cross-reactivity. Additionally, multiple antigen formats were used to ensure selection of clones that bind independently of post-translational modifications. Both NGS and ELISA primary screens were used to identify candidate binders. The resulting clones were initially ranked by affinity to human erythrocytes, then by affinity to cyno erythrocytes. Finally, binding kinetics to target protein from both species measured by Octet Bio-Layer Interferometry. The selection method is summarized in FIG. 5.

### Results

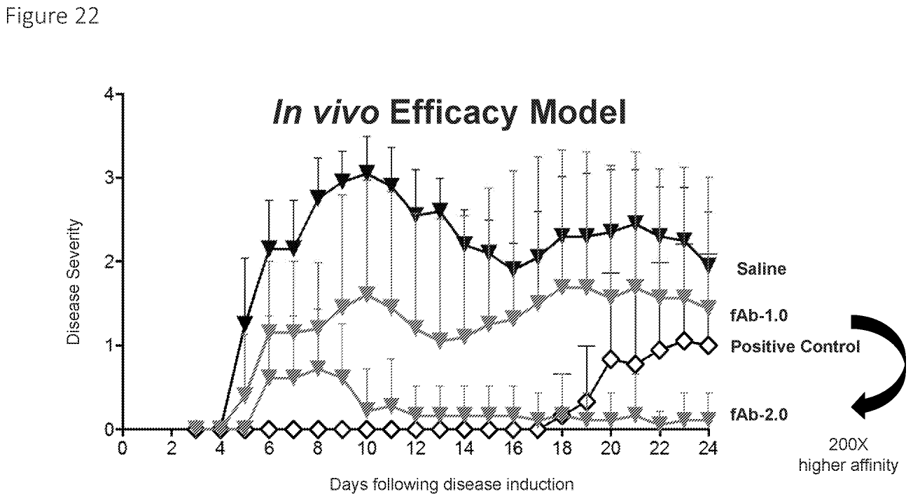

FIG. 22 depicts data relating to a correlation between affinity and tolerogenic response. As discussed above, an antigen, fragment thereof, or mimotope thereof can be attached to the binding moieties as disclosed herein. An antigen was fused to one of two different Fabs, each with different affinities for glycophorin A. Fab 2.0 has approximately 200× higher affinity for the target, as compared to the affinity of Fab 1.0. Disease severity was scored in animals after administration of the antigen-Fab fusions and used as a metric for an antigen-specific immune response. As shown in the Figure, Fab-2.0, with 200× higher affinity for erythrocytes, induced significant and sustained reduction in disease severity. In contrast, the lower affinity Fab (Fab 1.0) produced only modest reduction in disease severity. Thus, in accordance with several embodiments, the methods to screen and identify high affinity candidate binders is correlated with those binders yielding improved antigen-specific immune tolerance.

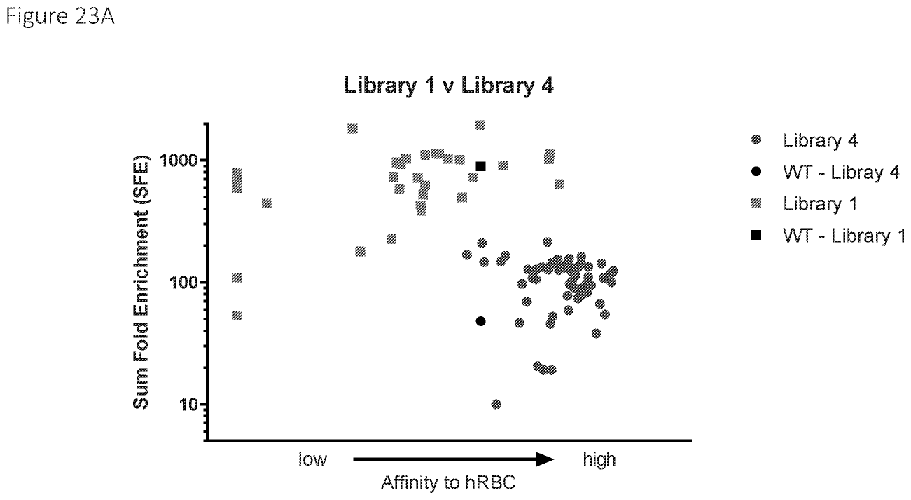

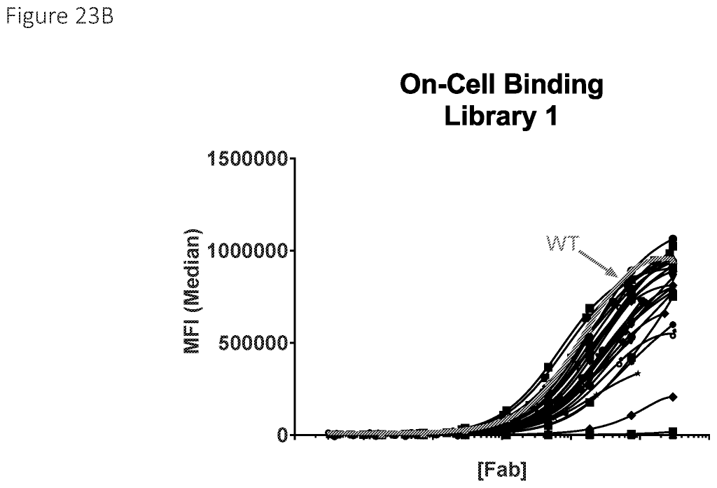

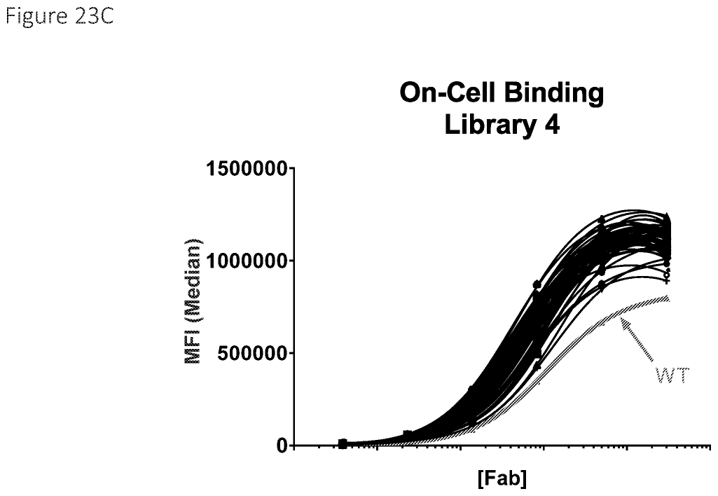

FIGS. 23A-23C relate to the type of sequences that can be enriched and also correlate to affinity improvements. FIG. 23A shows a plot of affinity of the clones tested vs sequence fold enrichment from Library 1 and Library 4. Library 1 was primarily enriched for Parental (WT or wild-type) and wild-type-like sequences. FIG. 23B shows the binding of the clones from Library 1 to human erythrocytes, illustrated as fluorescence intensity of Fab binding signal measured by flow cytometry. As shown, selection of this population of clones did not translate to affinity improvements to human erythrocytes (the plot of clones' binding affinity is right-shifted compared to the wild-type curve). In contrast, selections with Library 4 were successful, as show in FIG. 23C, wherein the flow cytometry affinity curves for the clones are left-shifted as compared to the wild-type curve, indicating higher affinity to human erythrocytes. This library produced clones with ˜6× higher affinity to human erythrocytes, as compared to the wild-type parental sequence.

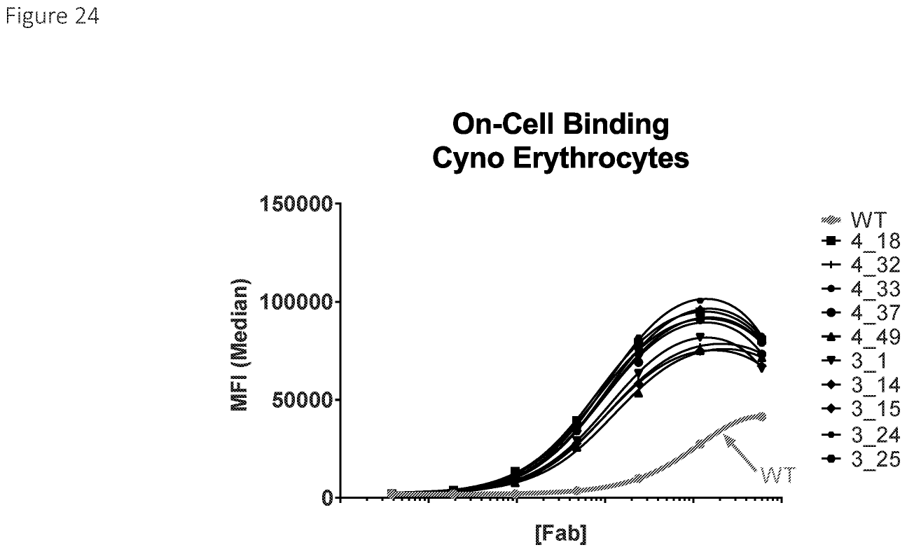

As discussed above, screening was performed with both human and cyno antigens, in order to select for clones with cross-reactivity to both species. FIG. 24 depicts data indicating that those clones that exhibited affinity improvements to hRBCs also showed affinity improvements to cyno (cRBCs). Flow cytometry was used to assess binding, with the top clones showing ˜16× higher affinity for cRBCs compared to wild-type. Advantageously, in several embodiments, such clones can be used for pre-clinical (e.g., primate studies) and human clinical trial work.

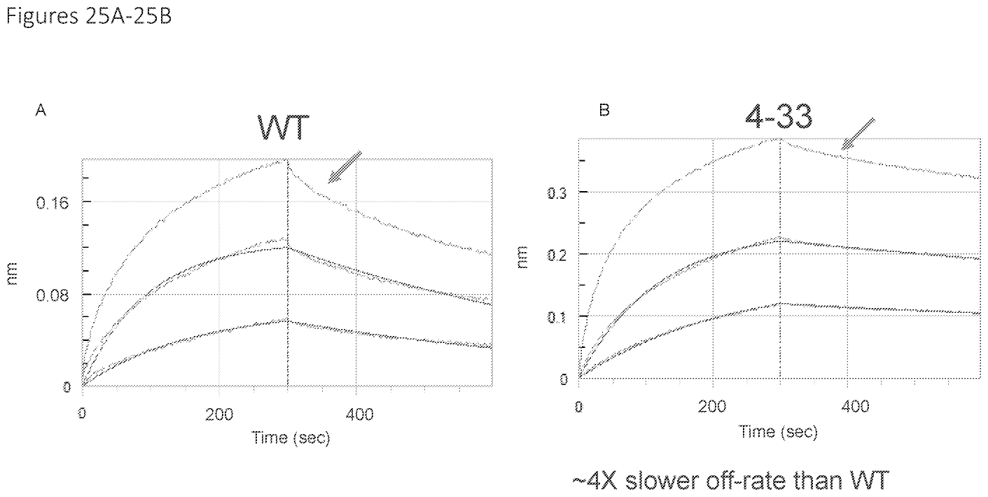

To further elucidate the mechanism underlying the improved affinity, Octet Bio-Layer Interferometry was used to evaluate binding kinetics. Sensograms of Fab binding kinetics are show in FIGS. 25A-25B. FIG. 25A shows the kinetic data for wild-type and FIG. 25B shows the data for a selected higher affinity clone. As depicted by the arrows, the off-rates differ between the two clones tested, with the off-rate of the higher affinity clone in 25B being ˜4× slower than wild-type. In several embodiments, the enhanced affinity can also be related to another kinetic characteristic, such as the association constant (e.g., the “on-rate” indicating how quickly a binder binds to its target).

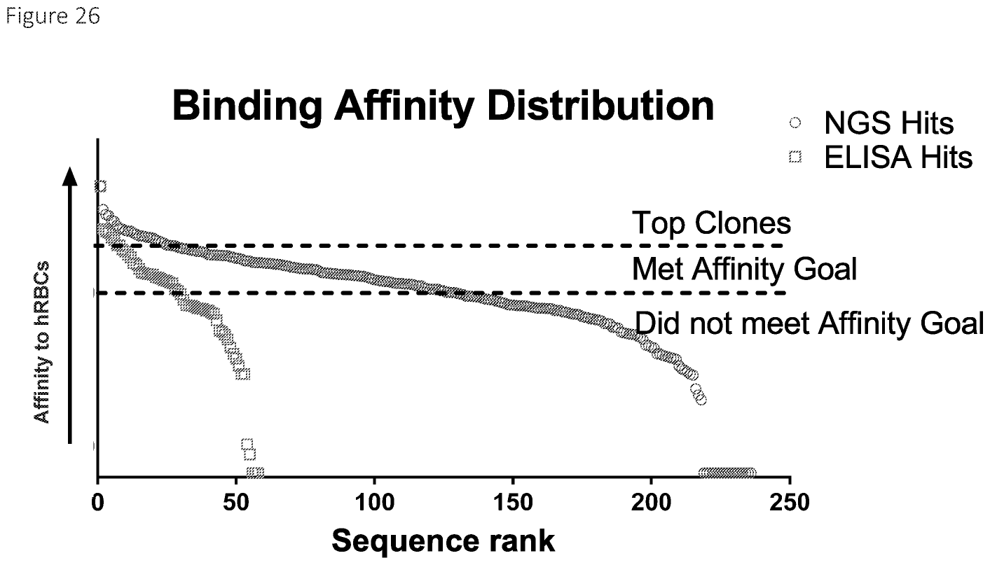

As discussed above in Example 3, an NGS primary screen advantageously provides a higher sampling rate of clones and wider range of affinities. Data from Example 3 above are shown again in FIG. 26, with a breakdown of the top performing clones, those that met the target affinity goals, and those that did not meet the affinity goals, broken down based on the screening method used. As shown in FIG. 26, and discussed above in Example 3, both ELISA and NGS screens were able to identify high affinity binders. Additionally, according to several embodiments, all, or substantially all, of the top ranking hits found by ELISA can also be identified by NGS. Advantageously, an NGS approach can reduce costs and time invested in a maturation campaign. Also, since the clones exhibiting the highest affinity were identified by both ELISA and NGS screen, a two-method quality control exercise can be implemented. Thus, Example 3 and 3A are non-limiting examples that demonstrate certain advantages of screening affinity matured candidates using and NGS-based methodology.

Additional non-limiting embodiments are described below. In several embodiments, there is provided an antigen-binding protein, comprising: a light chain complementary determining region (CDR)1 comprising RASSNVX₁X₂MY (SEQ ID NO: 49); and a light chain CDR2 comprising X₃X₄TSX₅LAS (SEQ ID NO: 50); wherein X₁, X₂, X₃, X₄, and X₅ are each a naturally occurring amino acid; wherein X₁ is not K when X₂ is Y, X₃ is Y, X₄ is Y, and X₅ is N; wherein X₂ is not Y when X₁ is K, X₃ is Y, X₄ is Y, and X₅ is N; wherein X₃ is not Y when X₁ is K, X₂ is Y, X₄ is Y, and X₅ is N; wherein X₄ is not Y when X₁ is K, X₂ is Y, X₃ is Y, and X₅ is N; and wherein X₅ is not N when X₁ is K, X₂ is Y, X₃ is Y, and X₄ is Y.

In several embodiments, the light chain CDR1 comprises RASSNVX₁X₂MY, wherein X₁ is selected from F, W, and Y, and wherein X₂ is selected from F, W, Y, and Q (SEQ ID NO: 51); and wherein the light chain CDR2 comprises X₃X₄TSX₅LAS, wherein X₃ is selected from H and Y, wherein X₄ is selected from H, R, and K, and wherein X₅ is a naturally occurring amino acid (SEQ ID NO: 52).

In several embodiments, the light chain CDR1 comprises RASSNVX₁X₂MY, wherein X₁ is selected from F, W, and Y, and wherein X₂ is selected from F, W, and Y (SEQ ID NO: 53); and wherein the light chain CDR2 comprises X₃X₄TSX₅LAS, wherein X₃ is H, wherein X₄ is H or R, and wherein X₅ is a naturally occurring amino acid (SEQ ID NO: 54).

In several embodiments, the light chain CDR1 comprises RASSNVX₁X₂MY, wherein X₁ is F or Y, and wherein X₂ is selected from F, W, and Y (SEQ ID NO: 55); and the light chain CDR2 comprises X₃X₄TSX₅LAS, wherein X₃ is H, wherein X₄ is H, and wherein X₅ is a naturally occurring amino acid (SEQ ID NO: 56).

In several embodiments, the light chain CDR1 comprises RASSNVX₁X₂MY, wherein X₁ is F or Y, and wherein X₂ is F, (SEQ ID NO: 57); and wherein the light chain CDR2 comprises X₃X₄TSX₅LAS, wherein X₃ is H, wherein X₄ is H, and wherein X₅ is a naturally occurring amino acid (SEQ ID NO: 56).

In several embodiments, the light chain CDR1 comprises RASSNVX₁X₂MY, wherein X₁ is F or Y, and wherein X₂ is F (SEQ ID NO: 57); and wherein the light chain CDR2 comprises X₃X₄TSX₅LAS, wherein X₃ is H, wherein X₄ is H, and wherein X₅ is V or D (SEQ ID NO: 58).

In several embodiments, there is provided an antigen-binding protein, comprising a light chain CDR1 comprising RASSNVX₁X₂MY (SEQ ID NO: 49); and a light chain CDR2 comprising X₃X₄TSX₅LAS (SEQ ID NO: 50); wherein X₁, X₂, X₃, X₄, and X₅ are each a naturally occurring amino acid.

In several embodiments, the light chain CDR1 comprises RASSNVX₁X₂MY, wherein X₁ is selected from F, W, and Y, and wherein X₂ is selected from F, W, Y, and Q (SEQ ID NO: 51); and wherein the light chain CDR2 comprises X₃X₄TSX₅LAS, wherein X₃ is selected from H and Y, wherein X₄ is selected from H, R, and K, and wherein X₅ is a naturally occurring amino acid (SEQ ID NO: 52).

In several embodiments, the light chain CDR1 comprises RASSNVX₁X₂MY, wherein X₁ is selected from F, W, and Y, and wherein X₂ is selected from F, W, and Y (SEQ ID NO: 53); and wherein the light chain CDR2 comprises X₃X₄TSX₅LAS, wherein X₃ is H, wherein X₄ is H or R, and wherein X₅ is a naturally occurring amino acid (SEQ ID NO: 54).

In several embodiments, the light chain CDR1 comprises RASSNVX₁X₂MY, wherein X₁ is F or Y, and wherein X₂ is selected from F, W, and Y (SEQ ID NO: 55); and the light chain CDR2 comprises X₃X₄TSX₅LAS, wherein X₃ is H, wherein X₄ is H, and wherein X₅ is a naturally occurring amino acid (SEQ ID NO: 56).

In several embodiments, the light chain CDR1 comprises RASSNVX₁X₂MY, wherein X₁ is F or Y, and wherein X₂ is F, (SEQ ID NO: 57); and wherein the light chain CDR2 comprises X₃X₄TSX₅LAS, wherein X₃ is H, wherein X₄ is H, and wherein X₅ is a naturally occurring amino acid (SEQ ID NO: 56).

In several embodiments, the light chain CDR1 comprises RASSNVX₁X₂MY, wherein X₁ is F or Y, and wherein X₂ is F (SEQ ID NO: 57); and wherein the light chain CDR2 comprises X₃X₄TSX₅LAS, wherein X₃ is H, wherein X₄ is H, and wherein X₅ is V or D (SEQ ID NO: 58).

In several embodiments, the antigen-binding protein further comprising a light chain CDR3 comprising QQFTSSPYT (SEQ ID NO: 45).

In several embodiments, there is provided an antigen-binding protein, comprising a light chain CDR1 comprising RASSNVX₁X₂MY (SEQ ID NO: 49), or a variant thereof comprising 1, 2, 3, or 4 conservative amino acid substitutions; and a light chain CDR2 comprising X₃X₄TSX₅LAS (SEQ ID NO: 50), or a variant thereof comprising 1, 2, 3, or 4 conservative amino acid substitutions; wherein X₁, X₂, X₃, X₄, and X₅ are each a naturally occurring amino acid.

In several embodiments, the antigen-binding protein light chain CDR1 comprises RASSNVX₁X₂MY, or a variant thereof comprising 1, 2, 3, or 4 conservative amino acid substitutions, wherein X₁ is selected from F, W, and Y, and wherein X₂ is selected from F, W, Y, and Q (SEQ ID NO: 51); and wherein the light chain CDR2 comprises X₃X₄TSX₅LAS (SEQ ID NO: 52), or a variant thereof comprising 1, 2, 3, or 4 conservative amino acid substitutions, wherein X₃ is selected from H and Y, wherein X₄ is selected from H, R, and K, and wherein X₅ is a naturally occurring amino acid (SEQ ID NO: 52).

In several embodiments, the light chain CDR1 comprises RASSNVX₁X₂MY, or a variant thereof comprising 1, 2, 3, or 4 conservative amino acid substitutions, wherein X₁ is selected from F, W, and Y, and wherein X₂ is selected from F, W, and Y (SEQ ID NO: 53); and wherein the light chain CDR2 comprises X₃X₄TSX₅LAS, or a variant thereof comprising 1, 2, 3, or 4 conservative amino acid substitutions wherein X₃ is H, wherein X₄ is H or R, and wherein X₅ is a naturally occurring amino acid (SEQ ID NO: 54).

In several embodiments, the light chain CDR1 comprises RASSNVX₁X₂MY, or a variant thereof comprising 1, 2, 3, or 4 conservative amino acid substitutions, wherein X₁ is F or Y, and wherein X₂ is selected from F, W, and Y (SEQ ID NO: 55); and wherein the light chain CDR2 comprises X₃X₄TSX₅LAS, or a variant thereof comprising 1, 2, 3, or 4 conservative amino acid substitutions, wherein X₃ is H, wherein X₄ is H, and wherein X₅ is a naturally occurring amino acid (SEQ ID NO: 56).

In several embodiments, the light chain CDR1 comprises RASSNVX₁X₂MY, or a variant thereof comprising 1, 2, 3, or 4 conservative amino acid substitutions, wherein X₁ is F or Y, and wherein X₂ is F (SEQ ID NO: 57); and wherein the light chain CDR2 comprises X₃X₄TSX₅LAS, or a variant thereof comprising 1, 2, 3, or 4 conservative amino acid substitutions, wherein X₃ is H, wherein X₄ is H, and wherein X₅ is a naturally occurring amino acid (SEQ ID NO: 56).

In several embodiments, the light chain CDR1 comprises RASSNVX₁X₂MY, or a variant thereof comprising 1, 2, 3, or 4 conservative amino acid substitutions, wherein X₁ is F or Y, and wherein X₂ is F (SEQ ID NO: 57); and wherein the light chain CDR2 comprises X₃X₄TSX₅LAS, or a variant thereof comprising 1, 2, 3, or 4 conservative amino acid substitutions, wherein X₃ is H, wherein X₄ is H, and wherein X₅ is V or D (SEQ ID NO: 58).

In several embodiments, the antigen-binding protein also includes a light chain CDR3 comprising QQFTSSPYT (SEQ ID NO: 45), or a variant thereof comprising 1, 2, 3, or 4 conservative amino acid substitutions.

In several embodiments, the antigen-binding protein also includes a heavy chain CDR1 comprising GYTFNSYFMH (SEQ ID NO: 46); a heavy chain CDR2 comprising GMIRPNGGTTDYNEKFKN (SEQ ID NO: 47); and a heavy chain CDR3 comprising WEGSYYALDY (SEQ ID NO: 48).

In several embodiments, the antigen-binding protein also includes a heavy chain CDR1 comprising GYTFNSYFMH (SEQ ID NO: 46), or a variant thereof comprising 1, 2, 3, or 4 conservative amino acid substitutions; a heavy chain CDR2 comprising GMIRPNGGTTDYNEKFKN (SEQ ID NO: 47), or a variant thereof comprising 1, 2, 3, or 4 conservative amino acid substitutions; and a heavy chain CDR3 comprising WEGSYYALDY (SEQ ID NO: 48), or a variant thereof comprising 1, 2, 3, or 4 conservative amino acid substitutions.

In several embodiments, the antigen-binding protein also includes a heavy chain variable domain that is at least about 90% identical to SEQ ID NO: 3. In several embodiments, the antigen-binding protein also includes a heavy chain variable domain that is at least about 95% identical to SEQ ID NO: 3. In several embodiments, the antigen-binding protein also includes a heavy chain variable domain that is at least about 99% identical to SEQ ID NO: 3.

In several embodiments, there is provided an antigen-binding protein comprising a heavy chain variable domain that is at least about 90% identical to SEQ ID NO: 3, and a light chain variable domain that is at least about 90% identical to SEQ ID NO: 1.

In several embodiments, there is provided an antigen-binding protein comprising a heavy chain variable domain that is at least about 95% identical to SEQ ID NO: 3, and a light chain variable domain that is at least about 95% identical to SEQ ID NO: 1.

In several embodiments, there is provided an antigen-binding protein comprising a heavy chain variable domain that is at least about 99% identical to SEQ ID NO: 3, and a light chain variable domain that is at least about 99% identical to SEQ ID NO: 1.

In several embodiments, there is provided an antigen-binding protein comprising an amino acid sequence selected from the group consisting of SEQ ID NOS: 4-42.

In several embodiments, there is provided an antigen-binding protein comprising an amino acid sequence selected from the group consisting of SEQ ID NOS: 5, 9, 11, 12, 14, 18, 19, 20, 21, 22, 24, 27, 28, 31, 38, and 42.

In several embodiments, there is provided an antigen-binding protein comprising the amino acid sequence of SEQ ID NO: 21. In several embodiments, there is provided an antigen-binding protein comprising the amino acid sequence of SEQ ID NO: 42. In several embodiments, the antigen-binding protein is humanized.

In several embodiments, the antigen-binding protein also includes (a) a first human light chain framework region (FR1) selected from SEQ ID NOS: 131-140, a human FR2 of the light chain framework region selected from SEQ ID NOS: 141-148, a human FR3 of the light chain selected from SEQ ID NOS: 149-156, and a human FR4 of the light chain selected from SEQ ID NOS: 157-162; and (b) a first human heavy chain framework region (FR1) selected from SEQ ID NOS: 109-115, a human FR2 of the heavy chain selected from SEQ ID NOS: 116-119, a human FR3 of the heavy chain selected from SEQ ID NOS: 120-127, and a human FR4 of the heavy chain selected from SEQ ID NOS: 128-130.

In several embodiments, the antigen-binding protein also includes a human constant region. In several embodiments, the constant region is selected from the group consisting of IgG1, IgG2, IgG3 and IgG4. In several embodiments, the constant region is IgG1.

In several embodiments, the antigen-binding protein is a full length antibody. In several embodiments, the antigen-binding protein is an antigen-binding fragment of an antibody.

In some embodiments, the antibody or antigen-binding fragment comprises a Fab, a Fab′, a F(ab′)2, a Fd, a single chain Fv or scFv, a disulfide linked Fv, a V NAR domain, a IgNar, an intrabody, an IgG-CH2, a minibody, a F(ab′)3, a tetrabody, a triabody, a diabody, a single-domain antibody, DVD-Ig, Fcab, mAb2, a (scFv)2, or a scFv-Fc.

In several embodiments, the antigen-binding protein specifically binds glycophorin A (GPA). In several embodiments, the antigen-binding protein the antigen-binding protein binds one or more of human GPA, cynomolgus GPA, porcine GPA, canine GPA, murine GPA, or rat GPA. In several embodiments, the antigen-binding protein specifically binds human GPA.

In several embodiments, the antigen-binding protein binds to human GPA with a K_{d} of about 1.0 to about 100 nM. In several embodiments, the K_{d} is about 1.0 nM or better (e.g., lower). In several embodiments, the binding affinity is measured by flow cytometry, surface plasmon resonance, biolayer inferometry, or radioimmunoassay.

In several embodiments, the antigen-binding protein is affinity matured. In several embodiments, the antigen-binding protein is an affinity matured variant of 10F7. In several embodiments, the antigen-binding protein competes for binding with GPA with 10F7, Ter119, CLB-ery-1 (AME-1), EPR8200, YTH89.1, EPR8199, JC159, GYPA/280, ab40844, HI264, GPHN02, JC159, SPM599, EPR8200, GYPA/1725R, ab112201, ab114330, ab219896, BRIC 256, or fragments or derivatives thereof. In several embodiments, the antigen-binding protein inhibits the binding of 10F7, Ter119, CLB-ery-1 (AME-1), EPR8200, YTH89.1, EPR8199, JC159, GYPA/280, ab40844, HI264, GPHN02, SPM599, GYPA/1725R, ab112201, BRIC 256, or fragments or derivatives thereof, to human GPA by at least about 50%.

In several embodiments, there is provided an antigen-binding protein that specifically binds human glycophorin A (GPA), comprising a light chain CDR1 comprising the amino acid sequence of any one of SEQ ID NO: 59, 61, 63, 65, 67, 69, 71, 73, 76, 78, 80, 81, 83, 85, 87, 88, 90, 91, 93, 95, 96, 98, 99, 101, 102, 104, 105, 107, and 108; a light chain CDR2 comprising the amino acid sequence of any one of SEQ ID NO: 60, 62, 64, 66, 68, 70, 72, 74, 75, 77, 79, 82, 84, 86, 89, 92, 94, 97, 100, 103, and 106; wherein the antigen-binding protein is affinity matured; and wherein the antigen-binding protein is humanized.

In several embodiments, there is provided an antigen-binding protein that specifically binds human glycophorin A (GPA), comprising (a) a light chain CDR1 comprising the amino acid sequence of any one of SEQ ID NO: 59, 61, 63, 65, 67, 69, 71, 73, 76, 78, 80, 81, 83, 85, 87, 88, 90, 91, 93, 95, 96, 98, 99, 101, 102, 104, 105, 107, and 108; (b) a light chain CDR2 comprising the amino acid sequence of any one of SEQ ID NO: 60, 62, 64, 66, 68, 70, 72, 74, 75, 77, 79, 82, 84, 86, 89, 92, 94, 97, 100, 103, and 106; (c) a light chain CDR3 comprising QQFTSSPYT (SEQ ID NO: 45), or a variant thereof comprising 1, 2, 3, or 4 conservative amino acid substitutions; (d) a heavy chain CDR1 comprising GYTFNSYFMH (SEQ ID NO: 46), or a variant thereof comprising 1, 2, 3, or 4 conservative amino acid substitutions; (e) a heavy chain CDR2 comprising GMIRPNGGTTDYNEKFKN (SEQ ID NO: 47), or a variant thereof comprising 1, 2, 3, or 4 conservative amino acid substitutions; (f) a heavy chain CDR3 comprising WEGSYYALDY (SEQ ID NO: 48), or a variant thereof comprising 1, 2, 3, or 4 conservative amino acid substitutions (g) a first human light chain framework region (FR1) selected from SEQ ID NOS: 131-140, a human FR2 of the light chain framework region selected from SEQ ID NOS: 141-148, a human FR3 of the light chain selected from SEQ ID NOS: 149-156, and a human FR4 of the light chain selected from SEQ ID NOS: 157-162; (h) a first human heavy chain framework region (FR1) selected from SEQ ID NOS: 109-115, a human FR2 of the heavy chain selected from SEQ ID NOS: 116-119, a human FR3 of the heavy chain selected from SEQ ID NOS: 120-127, and a human FR4 of the heavy chain selected from SEQ ID NOS: 128-130; (i) a human heavy chain constant region; and (j) a human light chain constant region.

In several embodiments, there is provided an isolated polynucleotide encoding the antigen-binding proteins disclosed herein.

In several embodiments, there is provided an isolated polynucleotide encoding a polypeptide at least 90% identical to an amino acid sequence selected from the group consisting of SEQ ID NOs: 1-42. In several embodiments, there is provided an isolated polynucleotide encoding a polypeptide at least 95% identical to an amino acid sequence selected from the group consisting of SEQ ID NOs: 1-42. In several embodiments, there is provided an isolated polynucleotide encoding a polypeptide at least 99% identical to an amino acid sequence selected from the group consisting of SEQ ID NOs: 1-42. In several embodiments, there is provided a vector comprising such polynucleotides. In several embodiments, there is provided a host cell comprising the vector. In several embodiments, the cell line is selected from the group consisting of CHO, k1SV, XCeed, CHOK1SV, and GS-KO.

In several embodiments, there is provided a method of producing an antigen-binding protein that specifically binds GPA, the method comprising culturing a cell line under conditions wherein the antigen-binding protein is produced; and recovering the antigen-binding protein. In several embodiments, the antigen-binding protein comprises one or more light chains and one or more heavy chains, and wherein the heavy and light chains are encoded on separate vectors. In several embodiments, the antigen-binding protein comprises one or more light chains and one or more heavy chains, and wherein the heavy and light chains are encoded on the same vectors. In several embodiments there is provided a pharmaceutical composition comprising an antigen-binding protein and a pharmaceutically acceptable carrier.

In several embodiments, there is provided a composition for inducing immune tolerance, comprising an antigen-binding protein and at least one immunogenic tolerogenic antigen or at least one immunogenic fragment of a tolerogenic antigen.

In several embodiments, the composition comprises at least one immunogenic tolerogenic antigen, wherein the at least one immunogenic tolerogenic antigen comprises at least one of myelin basic protein (MBP), myelin oligodendrocyte glycoprotein (MOG), myelin proteolipid protein (PLP), a fragment or fragments of MPB, a fragment or fragments of MOG, and a fragment or fragments of PLP. In several embodiments, the composition comprises at least one immunogenic fragment of a tolerogenic antigen, wherein the at least one immunogenic fragment of a tolerogenic antigen comprises at least one of SEQ ID Nos. 169-175, and 186-202. In several embodiments, the composition comprises at least one immunogenic fragment of a tolerogenic antigen, wherein the at least one immunogenic fragment of a tolerogenic antigen comprises at least one of SEQ ID Nos. 169, 171-173, 175, and 188-202. In several embodiments, the composition is for use in the treatment of multiple sclerosis. In several embodiments, a method of treating multiple sclerosis is provided comprising administering to a subject such a composition.

In several embodiments, the composition comprises at least one immunogenic tolerogenic antigen, wherein the at least one immunogenic tolerogenic antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, s loop, glial fibrillary acidic protein, regenerating gene II, pancreatic duodenal homeobox 1, dystrophia myotonica kinase, and SST G-protein coupled receptors 1-5. In several embodiments, the composition comprises at least one immunogenic fragment of a tolerogenic antigen, wherein the at least one immunogenic fragment of a tolerogenic antigen comprises SEQ ID Nos. 204-214. In several embodiments, the composition is for use in the treatment of Type 1 diabetes. In several embodiments, a method of treating Type 1 Diabetes is provided comprising administering to a subject such a composition.

In several embodiments, the composition comprises at least one immunogenic tolerogenic antigen, wherein the at least one immunogenic tolerogenic antigen comprises at least one of tissue transglutaminase, high molecular weight glutenin, low molecular weight glutenin, gluten, alpha-gliadin, gamma-gliadin, omega-gliadin, hordein, secalin, avenin, and deamidated forms thereof. In several embodiments, the composition comprises at least one immunogenic fragment of a tolerogenic antigen, wherein the at least one immunogenic fragment of a tolerogenic antigen comprises SEQ ID Nos. 182-185 and 215. In several embodiments, the composition is for use in the treatment of Celiac disease. In several embodiments, a method of treating celiac disease is provided comprising administering to a subject such a composition.

In several embodiments, the composition comprises at least one immunogenic tolerogenic antigen, wherein the at least one immunogenic tolerogenic antigen comprises at least one of a MHC class I protein, a MHC class II protein, minor blood group antigens, RhCE, Kell, Kidd, Duffy, and Ss. In several embodiments, the composition is for use in the prevention or treatment of transplant rejection. In several embodiments, a method of treating transplant rejection is provided comprising administering to a subject such a composition.

In several embodiments, use of compositions disclosed herein are for use in inducing immune tolerance to an antigen of interest, wherein the antigen of interest is coupled to the antigen-binding protein.

In several embodiments there is provided an antigen-binding protein, comprising a heavy chain complementary determining region 1 (CDR H1) comprising an amino acid sequence selected from RATYIL, RNIYIL, KYTYIL, VHTYIL, RNVFIL, RNIYLL, RKTYIL, LNVYIL, KATYIL, RMTYIL, KTVYIL, KHVYIL, RNITMIL, KDTYIL, INSYIL, QHTYIL, and RHSYIL. In several embodiments, the protein is matured from a parent sequence comprising a sequence of KDTYML at a corresponding location in the amino acid sequence. In several embodiments, the CDR H1 comprises a sequence selected from one of SEQ ID NOs: 217-233 or 269-285.

In several embodiments there is provided an antigen-binding protein, comprising a first light chain complementary determining region 1 (CDRL1a) comprising an amino acid sequence selected from FRNNK, FRNSK, FKNGK, FRNAK, FRTGK, FKNDK, and YKNGK. In several embodiments, the protein is matured from a parent sequence comprising a sequence of YSNGKT at a corresponding location in the amino acid sequence. In several embodiments, the CDRL1a comprises a sequence selected from one of SEQ ID NOs: 235-241 or 287-293.

In several embodiments there is provided an antigen-binding protein, comprising a light chain complementary determining region (CDRL2) comprising an amino acid sequence selected from LSRTS, NTRTS, NTRPS, LNRLH, NTRLA, NSRLS, LSRVS, LNRVS, LNRLS, NSRLH, SSRLS, SSRVS, SNRLH, NTRVS, SNRVS, HSRLS, SSRLA, FNRVN, LNRMS, LNRIS, and LSHPH. In several embodiments, the protein is matured from a parent sequence comprising a sequence of VSKLD at a corresponding location in the amino acid sequence. In several embodiments the CDRL2 comprises a sequence selected from one of SEQ ID NOs: 243-263 or 295-315.

In several embodiments, the antigen-binding protein binds a human target antigen and a cynomolgus target antigen. In several embodiments, the human target antigen and the cynomolgus target antigen are glycophorin A. In several embodiments, the antigen-binding protein is fused to an antigen against which tolerance is desired.

In several embodiments there is provided a composition for inducing immune tolerance, comprising an antigen-binding protein as disclosed herein and further comprising at least one immunogenic tolerogenic antigen or at least one immunogenic fragment of a tolerogenic antigen.

In several embodiments, the composition comprises at least one immunogenic tolerogenic antigen, wherein the at least one immunogenic tolerogenic antigen comprises at least one of myelin basic protein (MBP), myelin oligodendrocyte glycoprotein (MOG), myelin proteolipid protein (PLP), a fragment or fragments of MPB, a fragment or fragments of MOG, and a fragment or fragments of PLP. In several embodiments, the composition comprises at least one immunogenic fragment of a tolerogenic antigen, wherein the at least one immunogenic fragment of a tolerogenic antigen comprises at least one of SEQ ID Nos. 169-175, and 186-202. In several embodiments, the composition comprises at least one immunogenic fragment of a tolerogenic antigen, wherein the at least one immunogenic fragment of a tolerogenic antigen comprises at least one of SEQ ID Nos. 169, 171-173, 175, and 188-202. In several embodiments, the is for use in the treatment of multiple sclerosis.

In several embodiments, the composition comprises at least one immunogenic tolerogenic antigen, wherein the at least one immunogenic tolerogenic antigen comprises at least one of insulin, proinsulin, preproinsulin, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5. In several embodiments, the composition comprises at least one immunogenic fragment of a tolerogenic antigen, wherein the at least one immunogenic fragment of a tolerogenic antigen comprises SEQ ID Nos. 204-214. In several embodiments is for use in the treatment of Type I diabetes.

In several embodiments, the composition comprises at least one immunogenic tolerogenic antigen, wherein the at least one immunogenic tolerogenic antigen comprises at least one of tissue transglutaminase, high molecular weight glutenin, low molecular weight glutenin, gluten, alpha-gliadin, gamma-gliadin, omega-gliadin, hordein, secalin, avenin, and deamidated forms thereof. In several embodiments, the composition comprises at least one immunogenic fragment of a tolerogenic antigen, wherein the at least one immunogenic fragment of a tolerogenic antigen comprises SEQ ID Nos. 182-185 and 215. In several embodiments, the composition is for use in the treatment of Celiac disease.

In several embodiments, there are provided uses of the compositions disclosed herein for inducing immune tolerance to an antigen of interest, wherein the antigen of interest is fused to the antigen-binding protein.

In several embodiments, there is provided a method for affinity maturing an antibody or antibody fragment to have enhanced affinity for a target, the method comprising depleting a phage library of non-specific binders, wherein the phage library comprises a plurality of phage, each phage expressing a candidate affinity matured antibody or antibody fragment; exposing the depleted library to a target antigen; removing phage not bound to the target antigen by washing; amplifying the phage that are bound to target antigen; repeating the exposing, removing and amplifying steps a plurality of times to induce a selection pressure that results in binding of target antigen by phage expressing high affinity candidate antibodies or antibody fragments; and screening the high affinity candidate antibodies or antibody fragments using Next Generation Sequencing.

In several embodiments, the method optionally further comprising one or more of screening the high affinity candidate antibodies or antibody fragments using ELISA and evaluating the ability of the high affinity candidate antibodies or antibody fragments to be expressed in soluble form.

Although the foregoing has been described in some detail by way of illustrations and examples for purposes of clarity and understanding, it will be understood by those of skill in the art that modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an antigen-binding protein” include “instructing the administration of an antigen-binding protein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The term “about” as used herein to, for example, define the values and ranges of molecular weights means that the indicated values and/or range limits can vary within ±20%, e.g., within ±10%. The use of “about” before a number includes the number itself. For example, “about 5” provides express support for “5”. Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

1. An antigen-binding protein for binding to Glycophorin A, comprising:
a variable light (VL) domain comprising at least a first light chain complementary determining region (LC CDR1), a second light chain CDR (LC CDR2), and a third light chain CDR (LC CDR3);
a variable heavy (VH) domain comprising at least a first heavy chain complementary determining region (HC CDR1), a second heavy chain CDR (HC CDR2), and a third heavy chain CDR (HC CDR3),
wherein the antigen-binding protein comprises a fragment antibody binding (Fab), wherein the Fab binds to both human glycophorin A and cynomolgus glycophorin A,
wherein the antigen-binding protein binds to human glycophorin A with a dissociation constant (Kd) of about 1 nM to about 100 nM as measured by flow cytometry, surface plasmon resonance, biolayer inferometry, or radioimmunoassay, and
wherein the antigen-binding protein is affinity matured from a parent amino acid sequence encoding an antigen-binding protein having a greater Kd than the antigen-binding protein as measured by flow cytometry, surface plasmon resonance, biolayer inferometry, or radioimmunoassay.

2.-58. (canceled)

59. The antigen-binding protein of claim 1, wherein the Fab binds to human glycophorin A with a dissociation constant of between about 10 μM and 0.1 nM.

60. The antigen-binding protein of claim 1, wherein the LC CDR2 comprises an amino acid sequence selected from the group consisting of LNRLH (SEQ ID NO: 298), LSRTS (SEQ ID NO: 295), NTRTS (SEQ ID NO: 296), NTRPS (SEQ ID NO: 297), NTRLA (SEQ ID NO: 299), NSRLS (SEQ ID NO: 300), LSRVS (SEQ ID NO: 301), LNRVS (SEQ ID NO: 302), LNRLS (SEQ ID NO: 303), NSRLH (SEQ ID NO: 304), SSRLS (SEQ ID NO: 305), SSRVS (SEQ ID NO: 306), SNRLH (SEQ ID NO: 307), NTRVS (SEQ ID NO: 308), SNRVS (SEQ ID NO: 309), HSRLS (SEQ ID NO: 310), SSRLA (SEQ ID NO: 311), FNRVN (SEQ ID NO: 312), LNRMS (SEQ ID NO: 313), LNRIS (SEQ ID NO: 314), and LSHPH (SEQ ID NO: 315).

61. The antigen-binding protein of claim 60, wherein the VL domain comprises a sequence selected from any one of SEQ ID NOs: 246, 243, 244, 245, and 247-263.

62. The antigen-binding protein of claim 60, wherein the protein is matured from a parent sequence comprising a sequence of VSKLD at a corresponding location in the amino acid sequence of the VL domain.

63. The antigen-binding protein of claim 1, wherein the HC CDR1 comprises an amino acid sequence selected from the group consisting of RMTYIL (SEQ ID NO: 278), RATYIL (SEQ ID NO: 269), RNIYIL (SEQ ID NO: 270), KYTYIL (SEQ ID NO: 271), VHTYIL (SEQ ID NO: 272), RNVFIL (SEQ ID NO: 273), RNIYLI, (SEQ ID NO: 274), RKTYIL (SEQ ID NO: 275), LNVYIL (SEQ ID NO: 276), KATYIL (SEQ ID NO: 277), KTVYIL (SEQ ID NO: 279), KHVYIL (SEQ ID NO: 280), RNITMIL (SEQ ID NO: 281), KDTYIL (SEQ ID NO: 282), INSYIL (SEQ ID NO: 283), QHTYIL (SEQ ID NO: 284), and RHSYIL (SEQ ID NO: 285).

64. The antigen-binding protein of claim 63, wherein the VH domain comprises a sequence selected from any one of SEQ ID Nos: 227, 217-226, and 228-233.

65. The antigen-binding protein of claim 63, wherein the protein is matured from a parent sequence comprising a sequence of KDTYML at a corresponding location in the amino acid sequence.

66. The antigen-binding protein of claim 1, wherein the LC CDR1 comprises an amino acid sequence selected from FRNNK (SEQ ID NO: 287), FRNSK (SEQ ID NO: 288), FKNGK (SEQ ID NO: 289), FRNAK (SEQ ID NO: 290), FRTGK (SEQ ID NO: 291), FKNDK (SEQ ID NO: 292), and YKNGK (SEQ ID NO: 293).

67. The antigen-binding protein of claim 66, wherein the VL domain comprises a sequence selected from any one of SEQ ID NOs: 235-241.

68. The antigen-binding protein of claim 67, wherein the protein is matured from a parent sequence comprising a sequence of YSNGKT at a corresponding location in the amino acid sequence.

69. An antigen-binding protein for binding to Glycophorin A, comprising:
a variable light domain comprising at least a first light chain complementary determining region (LC CDR1) and a second light chain CDR (LC CDR2), and a third light chain CDR (LC CDR3);
a variable heavy domain comprising at least a first heavy chain complementary determining region (HC CDR1) and a second heavy chain CDR (HC CDR2), and a third heavy chain CDR (HC CDR3);
wherein the Fab binds to both human glycophorin A and cynomolgus glycophorin A, and
wherein the antigen-binding protein is affinity matured from a parent amino acid sequence encoding an antigen-binding protein.

70. The antigen-binding protein of claim 69, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with celiac disease, wherein the at least one immunogenic fragment of a tolerogenic antigen comprises at least one of SEQ ID NO: 183, SEQ ID NO: 215, SEQ ID NO: 182, SEQ ID NO:184, SEQ ID NO 185, an immunogenic fragment of one or more of: gluten, tissue transglutaminase, high molecular weight glutenin, low molecular weight glutenin, alpha-gliadin, gamma-gliadin, omega-gliadin, hordein, secalin, avenin, and/or deamidated forms thereof.

71. The antigen-binding protein of claim 69, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with multiple sclerosis, wherein the at least one immunogenic fragment of a tolerogenic antigen comprises at least one of SEQ ID NO: 173, SEQ ID NO: 174, SEQ ID NO: 187, SEQ ID NO: 189, SEQ ID NO: 200, SEQ ID NO: 201, SEQ ID NOs: 166-172, SEQ ID NO: 175, SEQ ID NO: 188, SEQ ID NOs: 190-199, SEQ ID NO: 202, an immunogenic fragment of myelin basic protein (MBP), an immunogenic fragment of myelin oligodendrocyte glycoprotein (MOG), and/or an immunogenic fragment of myelin proteolipid protein (PLP).

72. The antigen-binding protein of any one of claim 12, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with Type 1 Diabetes, wherein the antigen-binding protein is fused to at least one immunogenic fragment of a tolerogenic antigen, wherein the at least one immunogenic fragment of a tolerogenic antigen comprises an immunogenic fragment of one or more of: proinsulin, insulin, preproinsulin, SEQ ID Nos. 204-214, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5.

73. An antigen-binding protein comprising:
a light chain variable domain that is at least about 95% identical to one or more of SEQ ID NOs: 235-265;
a heavy chain variable domain that is at least about 95% identical to one or more of SEQ ID NOs: 217-234, and 267, and
wherein the antigen-binding protein binds to both human glycophorin A and cynomolgus glycophorin A.

74. The antigen-binding protein of claim 73, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with celiac disease, wherein the at least one immunogenic fragment of a tolerogenic antigen comprises at least one of SEQ ID NO: 183, SEQ ID NO: 215, SEQ ID NO: 182, SEQ ID NO:184, SEQ ID NO 185, an immunogenic fragment of one or more of: gluten, tissue transglutaminase, high molecular weight glutenin, low molecular weight glutenin, alpha-gliadin, gamma-gliadin, omega-gliadin, hordein, secalin, avenin, and/or deamidated forms thereof.

75. The antigen-binding protein of claim 73, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with multiple sclerosis, wherein the at least one immunogenic fragment of a tolerogenic antigen comprises at least one of SEQ ID NO: 173, SEQ ID NO: 174, SEQ ID NO: 187, SEQ ID NO: 189, SEQ ID NO: 200, SEQ ID NO: 201, SEQ ID NOs: 166-172, SEQ ID NO: 175, SEQ ID NO: 188, SEQ ID NOs: 190-199, SEQ ID NO: 202, an immunogenic fragment of myelin basic protein (MBP), an immunogenic fragment of myelin oligodendrocyte glycoprotein (MOG), and/or an immunogenic fragment of myelin proteolipid protein (PLP).

76. The antigen-binding protein of any one of claim 73, wherein the antigen-binding protein is fused to at least one immunogenic antigen or at least one immunogenic fragment of a tolerogenic antigen, wherein the at least one immunogenic tolerogenic antigen or at least one immunogenic fragment of a tolerogenic antigen is associated with Type 1 Diabetes, wherein the antigen-binding protein is fused to at least one immunogenic fragment of a tolerogenic antigen, wherein the at least one immunogenic fragment of a tolerogenic antigen comprises an immunogenic fragment of one or more of: proinsulin, insulin, preproinsulin, SEQ ID Nos. 204-214, glutamic acid decarboxylase-65 (GAD-65 or glutamate decarboxylase 2), GAD-67, glucose-6 phosphatase 2, islet-specific glucose 6 phosphatase catalytic subunit related protein (IGRP), insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5.

77. The antigen-binding protein of claim 73, wherein the antigen-binding protein binds to human glycophorin A with a dissociation constant of between about 10 μM and 0.1 nM.


SEQUENCE LISTING (316 sequences)

SEQ ID NO: 1 - protein, 106 aa, Homo sapiens
  MISC_FEATURE: 10F7-M10; Light Chain - variable region
EITLTQSPATLSLSPGERATLSCRASSNVKYMYWYQQKPGQAPRLWIYYTSNLASGIPDRFSGSGSGTDYTLTISRLEPEDFAVYYCQQFTSSPYTFGQGTKVEVK

SEQ ID NO: 2 - protein, 107 aa, Homo sapiens [sequence omitted]

SEQ ID NO: 3 - protein, 113 aa, Homo sapiens
  MISC_FEATURE: 10F7-M10; Heavy Chain - variable region
EVQLLESGGGLVQPGKSLRLSCKASGYTFNSYFMHWVRQAPGKGLEWVGMIRPNGGTTDYNEKFKNRFTLSVDKSKNTAYLQMNSLRAEDTAVYYCARWEGSYYALDYWGQGT

SEQ ID NO: 4 - protein, 103 aa, Homo sapiens
  MISC_FEATURE: 10F7-M10; Heavy Chain - constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 106 aa, Homo sapiens
  MISC_FEATURE: L1+2-1
EITLTQSPATLSLSPGERATLSCRASSNVFFMYWYQQKPGQAPRLWIHHTSNLASGIPDRFSGSGSGTDYTLTISRLEPEDFAVYYCQQFTSSPYTFGQGTKVEVK

SEQ ID NO: 6 - protein, 106 aa, Homo sapiens
  MISC_FEATURE: L1+2-2
EITLTQSPATLSLSPGERATLSCRASSNVEFMYWYQQKPGQAPRLWIFHTSELASGIPDRFSGSGSGTDYTLTISRLEPEDFAVYYCQQFTSSPYTFGQGTKVEVK

SEQ ID NO: 7 - protein, 106 aa, Homo sapiens
  MISC_FEATURE: L1+2-3
EITLTQSPATLSLSPGERATLSCRASSNVWGMYWYQQKPGQAPRLWIHRTSVLASGIPDRFSGSGSGTDYTLTISRLEPEDFAVYYCQQFTSSPYTFGQGTKVEVK

SEQ ID NO: 8 - protein, 106 aa, Homo sapiens
  MISC_FEATURE: L1+2-4
EITLTQSPATLSLSPGERATLSCRASSNVYDMYWYQQKPGQAPRLWIYRTSNLASGIPDRFSGSGSGTDYTLTISRLEPEDFAVYYCQQFTSSPYTFGQGTKVEVK

SEQ ID NO: 9 - protein, 106 aa, Homo sapiens
  MISC_FEATURE: L1+2-5
EITLTQSPATLSLSPGERATLSCRASSNVYNMYWYQQKPGQAPRLWIHRTSNLASGIPDRFSGSGSGTDYTLTISRLEPEDFAVYYCQQFTSSPYTFGQGTKVEVK

SEQ ID NO: 10 - protein, 106 aa, Homo sapiens
  MISC_FEATURE: L1+2-6
EITLTQSPATLSLSPGERATLSCRASSNVYFMYWYQQKPGQAPRLWIHHTSFLASGIPDRFSGSGSGTDYTLTISRLEPEDFAVYYCQQFTSSPYTFGQGTKVEVK

SEQ ID NO: 11 - protein, 106 aa, Homo sapiens
  MISC_FEATURE: L1+2-7
EITLTQSPATLSLSPGERATLSCRASSNVHHMYWYQQKPGQAPRLWIHHTSLLASGIPDRFSGSGSGTDYTLTISRLEPEDFAVYYCQQFTSSPYTFGQGTKVEVK

SEQ ID NO: 12 - protein, 106 aa, Homo sapiens
  MISC_FEATURE: L1+2-8
EITLTQSPATLSLSPGERATLSCRASSNVIDMYWYQQKPGQAPRLWIHHTSYLASGIPDRFSGSGSGTDYTLTISRLEPEDFAVYYCQQFTSSPYTFGQGTKVEVK

SEQ ID NO: 13 - protein, 106 aa, Homo sapiens
  MISC_FEATURE: L1+2-9
EITLTQSPATLSLSPGERATLSCRASSNVYFMYWYQQKPGQAPRLWIHHTSDLASGIPDRFSGSGSGTDYTLTISRLEPEDFAVYYCQQFTSSPYTFGQGTKVEVK

SEQ ID NO: 14 - protein, 106 aa, Homo sapiens
  MISC_FEATURE: L1+2-10
EITLTQSPATLSLSPGERATLSCRASSNVWFMYWYQQKPGQAPRLWIHHTSELASGIPDRFSGSGSGTDYTLTISRLEPEDFAVYYCQQFTSSPYTFGQGTKVEVK

SEQ ID NO: 15 - protein, 106 aa, Homo sapiens
  MISC_FEATURE: L1+2-11
EITLTQSPATLSLSPGERATLSCRASSNVRKMYWYQQKPGQAPRLWIHHTSTLASGIPDRFSGSGSGTDYTLTISRLEPEDFAVYYCQQFTSSPYTFGQGTKVEVK

SEQ ID NO: 16 - protein, 106 aa, Homo sapiens
  MISC_FEATURE: L1+2-12
EITLTQSPATLSLSPGERATLSCRASSNVAQMYWYQQKPGQAPRLWIHHTSDLASGIPDRFSGSGSGTDYTLTISRLEPEDFAVYYCQQFTSSPYTFGQGTKVEVK

SEQ ID NO: 17 - protein, 106 aa, Homo sapiens
  MISC_FEATURE: L1+2-13
EITLTQSPATLSLSPGERATLSCRASSNVVHMYWYQQKPGQAPRLWIAHTSELASGIPDRFSGSGSGTDYTLTISRLEPEDFAVYYCQQFTSSPYTFGQGTKVEVK

SEQ ID NO: 18 - protein, 106 aa, Homo sapiens
  MISC_FEATURE: L1+2-14
EITLTQSPATLSLSPGERATLSCRASSNVHQMYWYQQKPGQAPRLWIHHTSKLASGIPDRFSGSGSGTDYTLTISRLEPEDFAVYYCQQFTSSPYTFGQGTKVEVK

SEQ ID NO: 19 - protein, 106 aa, Homo sapiens
  MISC_FEATURE: L1+2-15
EITLTQSPATLSLSPGERATLSCRASSNVFYMYWYQQKPGQAPRLWIHHTSWLASGIPDRFSGSGSGTDYTLTISRLEPEDFAVYYCQQFTSSPYTFGQGTKVEVK

SEQ ID NO: 20 - protein, 106 aa, Homo sapiens
  MISC_FEATURE: L1+2-16
EITLTQSPATLSLSPGERATLSCRASSNVFQMYWYQQKPGQAPRLWIHHTSELASGIPDRFSGSGSGTDYTLTISRLEPEDFAVYYCQQFTSSPYTFGQGTKVEVK

SEQ ID NO: 21 - protein, 106 aa, Homo sapiens; identical to SEQ ID NO: 13
  MISC_FEATURE: L1+2-17

SEQ ID NO: 22 - protein, 106 aa, Homo sapiens
  MISC_FEATURE: L1+2-19
EITLTQSPATLSLSPGERATLSCRASSNVYQMYWYQQKPGQAPRLWIHHTSFLASGIPDRFSGSGSGTDYTLTISRLEPEDFAVYYCQQFTSSPYTFGQGTKVEVK

SEQ ID NO: 23 - protein, 106 aa, Homo sapiens
  MISC_FEATURE: L1+2-20
EITLTQSPATLSLSPGERATLSCRASSNVYFMYWYQQKPGQAPRLWIHHTSELASGIPDRFSGSGSGTDYTLTISRLEPEDFAVYYCQQFTSSPYTFGQGTKVEVK

SEQ ID NO: 24 - protein, 106 aa, Homo sapiens
  MISC_FEATURE: L1+2-21
EITLTQSPATLSLSPGERATLSCRASSNVYFMYWYQQKPGQAPRLWIHHTSVLASGIPDRFSGSGSGTDYTLTISRLEPEDFAVYYCQQFTSSPYTFGQGTKVEVK

SEQ ID NO: 25 - protein, 106 aa, Homo sapiens
  MISC_FEATURE: L1+2-22
EITLTQSPATLSLSPGERATLSCRASSNVHSMYWYQQKPGQAPRLWIHHTSYLASGIPDRFSGSGSGTDYTLTISRLEPEDFAVYYCQQFTSSPYTFGQGTKVEVK

SEQ ID NO: 26 - protein, 106 aa, Homo sapiens
  MISC_FEATURE: L1+2-23
EITLTQSPATLSLSPGERATLSCRASSNVWGMYWYQQKPGQAPRLWIHHTSELASGIPDRFSGSGSGTDYTLTISRLEPEDFAVYYCQQFTSSPYTFGQGTKVEVK

SEQ ID NO: 27 - protein, 106 aa, Homo sapiens
  MISC_FEATURE: L1+2-24
EITLTQSPATLSLSPGERATLSCRASSNVQQMYWYQQKPGQAPRLWIHHTSILASGIPDRFSGSGSGTDYTLTISRLEPEDFAVYYCQQFTSSPYTFGQGTKVEVK

SEQ ID NO: 28 - protein, 106 aa, Homo sapiens
  MISC_FEATURE: L1+2-25
EITLTQSPATLSLSPGERATLSCRASSNVAWMYWYQQKPGQAPRLWIYHTSKLASGIPDRFSGSGSGTDYTLTISRLEPEDFAVYYCQQFTSSPYTFGQGTKVEVK

SEQ ID NO: 29 - protein, 106 aa, Homo sapiens
  MISC_FEATURE: L1+2-26
EITLTQSPATLSLSPGERATLSCRASSNVSFMYWYQQKPGQAPRLWIHHTSKLASGIPDRFSGSGSGTDYTLTISRLEPEDFAVYYCQQFTSSPYTFGQGTKVEVK

SEQ ID NO: 30 - protein, 106 aa, Homo sapiens
  MISC_FEATURE: L1+2-27
EITLTQSPATLSLSPGERATLSCRASSNVYEMYWYQQKPGQAPRLWIHHTSQLASGIPDRFSGSGSGTDYTLTISRLEPEDFAVYYCQQFTSSPYTFGQGTKVEVK

SEQ ID NO: 31 - protein, 106 aa, Homo sapiens
  MISC_FEATURE: L1+2-28
EITLTQSPATLSLSPGERATLSCRASSNVYWMYWYQQKPGQAPRLWIHHTSDLASGIPDRFSGSGSGTDYTLTISRLEPEDFAVYYCQQFTSSPYTFGQGTKVEVK

SEQ ID NO: 32 - protein, 106 aa, Homo sapiens
  MISC_FEATURE: L1+2-29
EITLTQSPATLSLSPGERATLSCRASSNVSQMYWYQQKPGQAPRLWIHHTSGLASGIPDRFSGSGSGTDYTLTISRLEPEDFAVYYCQQFTSSPYTFGQGTKVEVK

SEQ ID NO: 33 - protein, 106 aa, Homo sapiens
  MISC_FEATURE: L1+2-30
EITLTQSPATLSLSPGERATLSCRASSNVTDMYWYQQKPGQAPRLWIHHTSWLASGIPDRFSGSGSGTDYTLTISRLEPEDFAVYYCQQFTSSPYTFGQGTKVEVK

SEQ ID NO: 34 - protein, 106 aa, Homo sapiens
  MISC_FEATURE: L1+2-31
EITLTQSPATLSLSPGERATLSCRASSNVDWMYWYQQKPGQAPRLWIHHTSFLASGIPDRFSGSGSGTDYTLTISRLEPEDFAVYYCQQFTSSPYTFGQGTKVEVK

SEQ ID NO: 35 - protein, 106 aa, Homo sapiens
  MISC_FEATURE: L1+2-32
EITLTQSPATLSLSPGERATLSCRASSNVRKMYWYQQKPGQAPRLWIHSTSGLASGIPDRFSGSGSGTDYTLTISRLEPEDFAVYYCQQFTSSPYTFGQGTKVEVK

SEQ ID NO: 36 - protein, 106 aa, Homo sapiens
  MISC_FEATURE: L1+2-33
EITLTQSPATLSLSPGERATLSCRASSNVASMYWYQQKPGQAPRLWIHRTSVLASGIPDRFSGSGSGTDYTLTISRLEPEDFAVYYCQQFTSSPYTFGQGTKVEVK

SEQ ID NO: 37 - protein, 106 aa, Homo sapiens
  MISC_FEATURE: L1+2-34
EITLTQSPATLSLSPGERATLSCRASSNVHGMYWYQQKPGQAPRLWIHHTSALASGIPDRFSGSGSGTDYTLTISRLEPEDFAVYYCQQFTSSPYTFGQGTKVEVK

SEQ ID NO: 38 - protein, 106 aa, Homo sapiens
  MISC_FEATURE: L1+2-35
EITLTQSPATLSLSPGERATLSCRASSNVYWMYWYQQKPGQAPRLWIHHTSELASGIPDRFSGSGSGTDYTLTISRLEPEDFAVYYCQQFTSSPYTFGQGTKVEVK

SEQ ID NO: 39 - protein, 106 aa, Homo sapiens
  MISC_FEATURE: L1+2-36
EITLTQSPATLSLSPGERATLSCRASSNVHHMYWYQQKPGQAPRLWIHHTSKLASGIPDRFSGSGSGTDYTLTISRLEPEDFAVYYCQQFTSSPYTFGQGTKVEVK

SEQ ID NO: 40 - protein, 106 aa, Homo sapiens
  MISC_FEATURE: L1+2-37
EITLTQSPATLSLSPGERATLSCRASSNVRGMYWYQQKPGQAPRLWIHHTSNLASGIPDRFSGSGSGTDYTLTISRLEPEDFAVYYCQQFTSSPYTFGQGTKVEVK

SEQ ID NO: 41 - protein, 106 aa, Homo sapiens
  MISC_FEATURE: L1+2-38
EITLTQSPATLSLSPGERATLSCRASSNVHEMYWYQQKPGQAPRLWIHHTSYLASGIPDRFSGSGSGTDYTLTISRLEPEDFAVYYCQQFTSSPYTFGQGTKVEVK

SEQ ID NO: 42 - protein, 106 aa, Homo sapiens
  MISC_FEATURE: L1+2-39
EITLTQSPATLSLSPGERATLSCRASSNVFFMYWYQQKPGQAPRLWIHHTSVLASGIPDRFSGSGSGTDYTLTISRLEPEDFAVYYCQQFTSSPYTFGQGTKVEVK

SEQ ID NO: 43 - protein, 10 aa, Homo sapiens
  MISC_FEATURE: 10F7-M10; CDR-L1
RASSNVKYMY

SEQ ID NO: 44 - protein, 8 aa, Homo sapiens
  MISC_FEATURE: 10F7-M10; CDR-L2
YYTSNLAS

SEQ ID NO: 45 - protein, 9 aa, Homo sapiens
  MISC_FEATURE: 10F7-M10; CDR-L3
QQFTSSPYT

SEQ ID NO: 46 - protein, 10 aa, Homo sapiens
  MISC_FEATURE: 10F7-M10; CDR-H1
GYTFNSYFMH

SEQ ID NO: 47 - protein, 18 aa, Homo sapiens
  MISC_FEATURE: 10F7-M10; CDR-H2
GMIRPNGGTTDYNEKFKN

SEQ ID NO: 48 - protein, 10 aa, Homo sapiens
  MISC_FEATURE: 10F7-M10; CDR-H3
WEGSYYALDY

SEQ ID NO: 49 - protein, 10 aa, Homo sapiens
  VARIANT (7)..(7): X is any naturally occurring amino acid
  MISC_FEATURE (7)..(7): light chain CDR1
  VARIANT (8)..(8): X is any naturally occurring amino acid
RASSNVXXMY

SEQ ID NO: 50 - protein, 8 aa, Homo sapiens
  variant (1)..(1): X is any naturally occuring amino acid
  MISC_FEATURE (1)..(1): light chain CDR2
  variant (2)..(2): X is any naturally occuring amino acid
  variant (5)..(5): X is any naturally occuring amino acid
XXTSXLAS

SEQ ID NO: 51 - protein, 10 aa, Homo sapiens
  MISC_FEATURE: light chain CDR1
  VARIANT (7)..(7): X is selected from F, W, and Y
  VARIANT (8)..(8): X is selected from F, W, Y, and Q
RASSNVXXMY

SEQ ID NO: 52 - protein, 8 aa, Homo sapiens
  MISC_FEATURE: light chain CDR2
  VARIANT (1)..(1): X is selected from H and Y
  VARIANT (2)..(2): X is selected from H, R, and K
  VARIANT (5)..(5): X is any naturally occurring amino acid
XXTSXLAS

SEQ ID NO: 53 - protein, 10 aa, Homo sapiens
  MISC_FEATURE: light chain CDR1
  VARIANT (7)..(7): X is selected from F, W and Y
  VARIANT (8)..(8): X is selected from F, W and Y
RASSNVXXMY

SEQ ID NO: 54 - protein, 8 aa, Homo sapiens
  MISC_FEATURE: light chain CDR2
  VARIANT (1)..(1): X is H
  VARIANT (2)..(2): X is H or R
  VARIANT (5)..(5): X is any naturally occuring amino acid
XXTSXLAS

SEQ ID NO: 55 - protein, 10 aa, Homo sapiens
  MISC_FEATURE: light chain CDR1
  VARIANT (7)..(7): X is F or Y
  VARIANT (8)..(8): X is F, W, or Y
RASSNVXXMY

SEQ ID NO: 56 - protein, 8 aa, Homo sapiens
  MISC_FEATURE: light chain CDR2
  VARIANT (1)..(1): X is H
  VARIANT (2)..(2): X is H
  VARIANT (5)..(5): X is any naturally occuring amino acid
XXTSXLAS

SEQ ID NO: 57 - protein, 10 aa, Homo sapiens
  MISC_FEATURE: light chain CDR1
  VARIANT (7)..(7): X is F or Y
  VARIANT (8)..(8): X is F
RASSNVXXMY

SEQ ID NO: 58 - protein, 8 aa, Homo sapiens
  MISC_FEATURE: light chain CDR2
  VARIANT (1)..(1): X is H
  VARIANT (2)..(2): X is H
  VARIANT (5)..(5): X is V or D
XXTSXLAS

SEQ ID NO: 59 - protein, 10 aa, Homo sapiens
  MISC_FEATURE: L1+2-1 CDR-L1
RASSNVFFMY

SEQ ID NO: 60 - protein, 8 aa, Homo sapiens
  MISC_FEATURE: L1+2-1 CDR-L2
HHTSNLAS

SEQ ID NO: 61 - protein, 10 aa, Homo sapiens
  MISC_FEATURE: L1+2-2 CDR-L1
RASSNVEFMY

SEQ ID NO: 62 - protein, 8 aa, Homo sapiens
  MISC_FEATURE: L1+2-2 CDR-L2
FHTSELAS

SEQ ID NO: 63 - protein, 10 aa, Homo sapiens
  MISC_FEATURE: L1+2-3 CDR-L1
RASSNVWGMY

SEQ ID NO: 64 - protein, 8 aa, Homo sapiens
  MISC_FEATURE: L1+2-3 CDR-L2
HRTSVLAS

SEQ ID NO: 65 - protein, 11 aa, Homo sapiens
  MISC_FEATURE: L1+2-4 CDR-L1
RASSNVYDMYW

SEQ ID NO: 66 - protein, 8 aa, Homo sapiens
  MISC_FEATURE: L1+2-4 CDR-L2
YRTSNLAS

SEQ ID NO: 67 - protein, 10 aa, Homo sapiens
  MISC_FEATURE: L1+2-5 CDR-L1
RASSNVYNMY

SEQ ID NO: 68 - protein, 8 aa, Homo sapiens
  MISC_FEATURE: L1+2-5 CDR-L2
HRTSNLAS

SEQ ID NO: 69 - protein, 10 aa, Homo sapiens
  MISC_FEATURE: L1+2-6 CDR-L1
RASSNVYFMY

SEQ ID NO: 70 - protein, 8 aa, Homo sapiens
  MISC_FEATURE: L1+2-6 CDR-L2
HHTSFLAS

SEQ ID NO: 71 - protein, 10 aa, Homo sapiens
  MISC_FEATURE: L1+2-7 CDR-L1
RASSNVHHMY

SEQ ID NO: 72 - protein, 8 aa, Homo sapiens
  MISC_FEATURE: L1+2-7 CDR-L2
HHTSLLAS

SEQ ID NO: 73 - protein, 10 aa, Homo sapiens
  MISC_FEATURE: L1+2-8 CDR-L1
RASSNVIDMY

SEQ ID NO: 74 - protein, 8 aa, Homo sapiens
  MISC_FEATURE: L1+2-8 CDR-L2
HHTSYLAS

SEQ ID NO: 75 - protein, 8 aa, Homo sapiens
  MISC_FEATURE: L1+2-9 CDR-L2
HHTSDLAS

SEQ ID NO: 76 - protein, 10 aa, Homo sapiens
  MISC_FEATURE: L1+2-10 CDR-L1
RASSNVWFMY

SEQ ID NO: 77 - protein, 8 aa, Homo sapiens
  MISC_FEATURE: L1+2-10 CDR-L2
HHTSELAS

SEQ ID NO: 78 - protein, 10 aa, Homo sapiens
  MISC_FEATURE: L1+2-11 CDR-L1
RASSNVRKMY

SEQ ID NO: 79 - protein, 8 aa, Homo sapiens
  MISC_FEATURE: L1+2-11 CDR-L2
HHTSTLAS

SEQ ID NO: 80 - protein, 10 aa, Homo sapiens
  MISC_FEATURE: L1+2-12 CDR-L1
RASSNVAQMY

SEQ ID NO: 81 - protein, 10 aa, Homo sapiens
  MISC_FEATURE: L1+2-13 CDR-L1
RASSNVVHMY

SEQ ID NO: 82 - protein, 8 aa, Homo sapiens
  MISC_FEATURE: L1+2-13 CDR-L2
AHTSELAS

SEQ ID NO: 83 - protein, 10 aa, Homo sapiens
  MISC_FEATURE: L1+2-14 CDR-L1
RASSNVHQMY

SEQ ID NO: 84 - protein, 8 aa, Homo sapiens
  MISC_FEATURE: L1+2-14 CDR-L2
HHTSKLAS

SEQ ID NO: 85 - protein, 10 aa, Homo sapiens
  MISC_FEATURE: L1+2-15 CDR-L1
RASSNVFYMY

SEQ ID NO: 86 - protein, 8 aa, Homo sapiens
  MISC_FEATURE: L1+2-15 CDR-L2
HHTSWLAS

SEQ ID NO: 87 - protein, 10 aa, Homo sapiens
  MISC_FEATURE: L1+2-16 CDR-L1
RASSNVFQMY

SEQ ID NO: 88 - protein, 10 aa, Homo sapiens
  MISC_FEATURE: L1+2-19 CDR-L1
RASSNVYQMY

SEQ ID NO: 89 - protein, 8 aa, Homo sapiens
  MISC_FEATURE: L1+2-21 CDR-L2
HHTSVLAS

SEQ ID NO: 90 - protein, 10 aa, Homo sapiens
  MISC_FEATURE: L1+2-22 CDR-L1
RASSNVHSMY

SEQ ID NO: 91 - protein, 10 aa, Homo sapiens
  MISC_FEATURE: L1+2-24 CDR-L1
RASSNVQQMY

SEQ ID NO: 92 - protein, 8 aa, Homo sapiens
  MISC_FEATURE: L1+2-24 CDR-L2
HHTSILAS

SEQ ID NO: 93 - protein, 9 aa, Homo sapiens
  MISC_FEATURE: L1+2-25 CDR-L1
RASSNVAWM

SEQ ID NO: 94 - protein, 8 aa, Homo sapiens
  MISC_FEATURE: L1+2-25 CDR-L2
YHTSKLAS

SEQ ID NO: 95 - protein, 10 aa, Homo sapiens
  MISC_FEATURE: L1+2-26 CDR-L1
RASSNVSFMY

SEQ ID NO: 96 - protein, 10 aa, Homo sapiens
  MISC_FEATURE: L1+2-27 CDR-L1
RASSNVYEMY

SEQ ID NO: 97 - protein, 8 aa, Homo sapiens
  MISC_FEATURE: L1+2-27 CDR-L2
HHTSQLAS

SEQ ID NO: 98 - protein, 10 aa, Homo sapiens
  MISC_FEATURE: L1+2-28 CDR-L1
RASSNVYWMY

SEQ ID NO: 99 - protein, 9 aa, Homo sapiens
  MISC_FEATURE: L1+2-29 CDR-L1
RASSNVSQM

SEQ ID NO: 100 - protein, 8 aa, Homo sapiens
  MISC_FEATURE: L1+2-29 CDR-L2
HHTSGLAS

SEQ ID NO: 101 - protein, 10 aa, Homo sapiens
  MISC_FEATURE: L1+2-30 CDR-L1
RASSNVTDMY

SEQ ID NO: 102 - protein, 10 aa, Homo sapiens
  MISC_FEATURE: L1+2-31 CDR-L1
RASSNVDWMY

SEQ ID NO: 103 - protein, 8 aa, Homo sapiens
  MISC_FEATURE: L1+2-32 CDR-L2
HSTSGLAS

SEQ ID NO: 104 - protein, 10 aa, Homo sapiens
  MISC_FEATURE: L1+2-33 CDR-L1
RASSNVASMY

SEQ ID NO: 105 - protein, 10 aa, Homo sapiens
  MISC_FEATURE: L1+2-34 CDR-L1
RASSNVHGMY

SEQ ID NO: 106 - protein, 8 aa, Homo sapiens
  MISC_FEATURE: L1+2-34 CDR-L2
HHTSALAS

SEQ ID NO: 107 - protein, 10 aa, Homo sapiens
  MISC_FEATURE: L1+2-37 CDR-L1
RASSNVRGMY

SEQ ID NO: 108 - protein, 10 aa, Homo sapiens
  MISC_FEATURE: L1+2-38 CDR-L1
RASSNVHEMY

SEQ ID NO: 109 - protein, 25 aa, Homo sapiens
  MISC_FEATURE: Heavy Chain FR1
EVQLLESGGGLVQPGKSLRLSCKAS

SEQ ID NO: 110 - protein, 25 aa, Homo sapiens [sequence omitted]

SEQ ID NO: 111 - protein, 25 aa, Homo sapiens [sequence omitted]

SEQ ID NO: 112 - protein, 25 aa, Homo sapiens
  MISC_FEATURE: Heavy Chain FR1
EVQLVQSGAEVKKPGATVKISCKVS

SEQ ID NO: 113 - protein, 25 aa, Homo sapiens
  MISC_FEATURE: Heavy Chain FR1
EVQLVQSGAEVKKPGESLKISCKGS

SEQ ID NO: 114 - protein, 25 aa, Homo sapiens
  MISC_FEATURE: Heavy Chain FR1
EVQLVQSGAEVKKPGESLRISCKGS

SEQ ID NO: 115 - protein, 25 aa, Homo sapiens
  MISC_FEATURE: Heavy Chain FR1
QVQLVQSGSELKKPGASVKVSCKAS

SEQ ID NO: 116 - protein, 13 aa, Homo sapiens
  MISC_FEATURE: Heavy Chain FR2
WVRQAPGKGLEWV

SEQ ID NO: 117 - protein, 13 aa, Homo sapiens
  MISC_FEATURE: Heavy Chain FR2
WVRQAPGQGLEWM

SEQ ID NO: 118 - protein, 13 aa, Homo sapiens
  MISC_FEATURE: Heavy Chain FR2
WVQQAPGKGLEWM

SEQ ID NO: 119 - protein, 13 aa, Homo sapiens
  MISC_FEATURE: Heavy Chain FR2
WVRQMPGKGLEWM

SEQ ID NO: 120 - protein, 32 aa, Homo sapiens
  MISC_FEATURE: Heavy Chain FR3
RFTLSVDKSKNTAYLQMNSLRAEDTAVYYCAR

SEQ ID NO: 121 - protein, 32 aa, Homo sapiens
  MISC_FEATURE: Heavy Chain FR3
RVTMTTDTSTSTAYMELRSLRSDDTAVYYCAR

SEQ ID NO: 122 - protein, 32 aa, Homo sapiens
  MISC_FEATURE: Heavy Chain FR3
RVTITADESTSTAYMELSSLRSEDTAVYYCAR

SEQ ID NO: 123 - protein, 32 aa, Homo sapiens
  MISC_FEATURE: Heavy Chain FR3
RVTITADKSTSTAYMELSSLRSEDTAVYYCAR

SEQ ID NO: 124 - protein, 32 aa, Homo sapiens
  MISC_FEATURE: Heavy Chain FR3
RVTITADTSTDTAYMELSSLRSEDTAVYYCAT

SEQ ID NO: 125 - protein, 32 aa, Homo sapiens
  MISC_FEATURE: Heavy Chain FR3
QVTISADKSISTAYLQWSSLKASDTAMYYCAR

SEQ ID NO: 126 - protein, 32 aa, Homo sapiens
  MISC_FEATURE: Heavy Chain FR3
HVTISADKSISTAYLQWSSLKASDTAMYYCAR

SEQ ID NO: 127 - protein, 32 aa, Homo sapiens
  MISC_FEATURE: Heavy Chain FR3
RFVFSLDTSVSTAYLQISSLKAEDTAVYYCAR

SEQ ID NO: 128 - protein, 5 aa, Homo sapiens
  MISC_FEATURE: Heavy Chain FR4
WGQGT

SEQ ID NO: 129 - protein, 5 aa, Homo sapiens
  MISC_FEATURE: Heavy Chain FR4
WGRGT

SEQ ID NO: 130 - protein, 5 aa, Homo sapiens
  MISC_FEATURE: Heavy Chain FR4
WGKGT

SEQ ID NO: 131 - protein, 23 aa, Homo sapiens
  MISC_FEATURE: Light Chain FR1
EITLTQSPATLSLSPGERATLSC

SEQ ID NO: 132 - protein, 23 aa, Homo sapiens
  MISC_FEATURE: Light Chain FR1
EIVMTQSPATLSVSPGERATLSC

SEQ ID NO: 133 - protein, 23 aa, Homo sapiens
  MISC_FEATURE: Light Chain FR1
EIVLTQSPATLSLSPGERATLSC

SEQ ID NO: 134 - protein, 23 aa, Homo sapiens
  MISC_FEATURE: Light Chain FR1
DIQMTQSPSSLSASVGDRVTITC

SEQ ID NO: 135 - protein, 23 aa, Homo sapiens
  MISC_FEATURE: Light Chain FR1
NIQMTQSPSAMSASVGDRVTITC

SEQ ID NO: 136 - protein, 23 aa, Homo sapiens
  MISC_FEATURE: Light Chain FR1
AIQLTQSPSSLSASVGDRVTITC

SEQ ID NO: 137 - protein, 23 aa, Homo sapiens
  MISC_FEATURE: Light Chain FR1
DIQLTQSPSFLSASVGDRVTITC

SEQ ID NO: 138 - protein, 23 aa, Homo sapiens
  MISC_FEATURE: Light Chain FR1
AIRMTQSPFSLSASVGDRVTITC

SEQ ID NO: 139 - protein, 23 aa, Homo sapiens
  MISC_FEATURE: Light Chain FR1
AIQMTQSPSSLSASVGDRVTITC

SEQ ID NO: 140 - protein, 23 aa, Homo sapiens
  MISC_FEATURE: Light Chain FR1
DIQMTQSPSTLSASVGDRVTITC

SEQ ID NO: 141 - protein, 14 aa, Homo sapiens
  MISC_FEATURE: Light Chain FR2
WYQQKPGQAPRLWI

SEQ ID NO: 142 - protein, 14 aa, Homo sapiens
  MISC_FEATURE: Light Chain FR2
WYQQKPGQAPRLLI

SEQ ID NO: 143 - protein, 14 aa, Homo sapiens
  MISC_FEATURE: Light Chain FR2
WYQQKPGKAPKLLI

SEQ ID NO: 144 - protein, 14 aa, Homo sapiens
  MISC_FEATURE: Light Chain FR2
WYQQKPGKVPKLLI

SEQ ID NO: 145 - protein, 14 aa, Homo sapiens
  MISC_FEATURE: Light Chain FR2
WYQQKPGKAPKRLI

SEQ ID NO: 146 - protein, 14 aa, Homo sapiens
  MISC_FEATURE: Light Chain FR2
WFQQKPGKVPKHLI

SEQ ID NO: 147 - protein, 14 aa, Homo sapiens
  MISC_FEATURE: Light Chain FR2
WFQQKPGKAPKSLI

SEQ ID NO: 148 - protein, 14 aa, Homo sapiens
  MISC_FEATURE: Light Chain FR2
WYQQKPAKAPKLFI

SEQ ID NO: 149 - protein, 32 aa, Homo sapiens
  MISC_FEATURE: Light Chain FR3
GIPDRFSGSGSGTDYTLTISRLEPEDFAVYYC

SEQ ID NO: 150 - protein, 32 aa, Homo sapiens
  MISC_FEATURE: Light Chain FR3
GIPARFSGSGSGTEFTLTISSLQSEDFAVYYC

SEQ ID NO: 151 - protein, 32 aa, Homo sapiens
  MISC_FEATURE: Light Chain FR3
GIPARFSGSGSGTDFTLTISSLEPEDFAVYYC

SEQ ID NO: 152 - protein, 32 aa, Homo sapiens
  MISC_FEATURE: Light Chain FR3
GVPSRFSGSGSGTDFTLTISSLQPEDFATYYC

SEQ ID NO: 153 - protein, 32 aa, Homo sapiens
GVPSRFSGSGSGTDFTLTISSLQPEDVATYYC

SEQ ID NO: 154 - protein, 32 aa, Homo sapiens
  MISC_FEATURE: Light Chain FR3
GVPSRFSGSGSGTEFTLTISSLQPEDFATYYC

SEQ ID NO: 155 - protein, 32 aa, Homo sapiens
  MISC_FEATURE: Light Chain FR3
GVPSRFSGSGSGTDYTLTISSLQPEDFATYYC

SEQ ID NO: 156 - protein, 32 aa, Homo sapiens
  MISC_FEATURE: Light Chain FR3
GVPSRFSGSGSGTEFTLTISSLQPDDFATYYC

SEQ ID NO: 157 - protein, 10 aa, Homo sapiens
  MISC_FEATURE: Light Chain FR4
FGQGTKVEVK

SEQ ID NO: 158 - protein, 10 aa, Homo sapiens
  MISC_FEATURE: Light Chain FR4
FGQGTKVEIK

SEQ ID NO: 159 - protein, 10 aa, Homo sapiens
  MISC_FEATURE: Light Chain FR4
FGQGTKLEIK

SEQ ID NO: 160 - protein, 10 aa, Homo sapiens
  MISC_FEATURE: Light Chain FR4
FGPGTKVDIK

SEQ ID NO: 161 - protein, 10 aa, Homo sapiens
  MISC_FEATURE: Light Chain FR4
FGGGTKVEIK

SEQ ID NO: 162 - protein, 10 aa, Homo sapiens
  MISC_FEATURE: Light Chain FR4
FGQGTRLEIK

SEQ ID NO: 163 - protein, 110 aa, Homo sapiens
  MISC_FEATURE: Human insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 164 - protein, 585 aa, Homo sapiens
  MISC_FEATURE: GAD65
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 165 - protein, 355 aa, Homo sapiens
  MISC_FEATURE: IGRP
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 166 - protein, 304 aa, Homo sapiens
  MISC_FEATURE: Myelin Basic Protein
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 167 - protein, 247 aa, Homo sapiens
  MISC_FEATURE: Myelin Oligodendrocyte Protein
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 168 - protein, 277 aa, Homo sapiens
  MISC_FEATURE: Proteolipid Protein
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 169 - protein, 20 aa, Homo sapiens
  MISC_FEATURE: MBP13-32
KYLATASTMDHARHGFLPRH

SEQ ID NO: 170 - protein, 16 aa, Homo sapiens
  MISC_FEATURE: MBP83-99
ENPWHFFKNIVTPRTP

SEQ ID NO: 171 - protein, 19 aa, Homo sapiens
  MISC_FEATURE: MBP111-129
LSRFSWGAEGQRPGFGYGG

SEQ ID NO: 172 - protein, 25 aa, Homo sapiens
  MISC_FEATURE: MBP146-170
AQGTLSKIFKLGGRDSRSGSPMARR

SEQ ID NO: 173 - protein, 20 aa, Homo sapiens
  MISC_FEATURE: MOG1-20
GQFRVIGPRHPIRALVGDEV

SEQ ID NO: 174 - protein, 16 aa, Homo sapiens
  MISC_FEATURE: PLP139-154
HCLGKWLGHPDKFVGI

SEQ ID NO: 175 - protein, 118 aa, Homo sapiens
  MISC_FEATURE: MART1
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 176 - protein, 529 aa, Homo sapiens [sequence omitted]

SEQ ID NO: 177 - protein, 661 aa, Homo sapiens
  MISC_FEATURE: Melanocyte protein PMEL, gp100
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 178 - protein, 323 aa, Homo sapiens
  MISC_FEATURE: Aquaporin-4
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 179 - protein, 405 aa, Homo sapiens
  MISC_FEATURE: S-arrestin
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 180 - protein, 1247 aa, Homo sapiens
  MISC_FEATURE: IRBP
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 181 - protein, 33 aa, Homo sapiens
  MISC_FEATURE: DQ-2 relevant, Alpha-gliadin “33-mer” native
LQLQPFPQPQLPYPQPQLPYPQPQLPYPQPQPF

SEQ ID NO: 182 - protein, 33 aa, Homo sapiens
  MISC_FEATURE: DQ-2 relevant, Alpha-gliadin “33-mer” deamidated
LQLQPFPQPELPYPQPELPYPQPELPYPQPQPF

SEQ ID NO: 183 - protein, 18 aa, Homo sapiens
  MISC_FEATURE: DQ-8 relevant, Alpha-gliadin
QQYPSGQGSFQPSQQNPQ

SEQ ID NO: 184 - protein, 12 aa, Homo sapiens
  MISC_FEATURE: DQ-8 relevant, Omega-gliadin (wheat, U5UA46)
QPFPQPEQPFPW

SEQ ID NO: 185 - protein, 21 aa, Homo sapiens
  MISC_FEATURE: MOG35-55
MEVGWYRSPFSRVVHLYRNGK

SEQ ID NO: 186 - protein, 31 aa, Homo sapiens
  MISC_FEATURE: MOG30-60
KNATGMEVGWYRSPFSRVVHLYRNGKDQDAE

SEQ ID NO: 187 - protein, 20 aa, Homo sapiens
  MISC_FEATURE: MOG35-55
MEVGWYRPPFSRWHLYRNGK

SEQ ID NO: 188 - protein, 17 aa, Homo sapiens
  MISC_FEATURE: MOG83-99
ENPVVHFFKNIVTPRTP

SEQ ID NO: 189 - protein, 21 aa, Homo sapiens
  MISC_FEATURE: MOG35-55
MEVGWYRPPFSRVVHLYRNGK

SEQ ID NO: 190 - protein, 17 aa, Homo sapiens
  MISC_FEATURE: MBP82-98
DENPVVHFFKNIVTPRT

SEQ ID NO: 191 - protein, 18 aa, Homo sapiens
  MISC_FEATURE: MBP82-99
DENPVVHFFKNIVTPRTP

SEQ ID NO: 192 - protein, 25 aa, Homo sapiens
  MISC_FEATURE: MBP82-106
DENPVVHFFKNIVTPRTPPPSQGKG

SEQ ID NO: 193 - protein, 20 aa, Homo sapiens
  MISC_FEATURE: MBP87-106
VHFFKNIVTPRTPPPSQGKG

SEQ ID NO: 194 - protein, 25 aa, Homo sapiens
  MISC_FEATURE: MBP131-155
ASDYKSAHKGLKGVDAQGTLSKIFK

SEQ ID NO: 195 - protein, 18 aa, Homo sapiens
  MISC_FEATURE: PLP41-58
GTEKLIETYFSKNYQDYE

SEQ ID NO: 196 - protein, 18 aa, Homo sapiens
  MISC_FEATURE: PLP89-106
GFYTTGAVRQIFGDYKTT

SEQ ID NO: 197 - protein, 22 aa, Homo sapiens
  MISC_FEATURE: PLP95-116
AVRQIFGDYKTTICGKGLSATV

SEQ ID NO: 198 - protein, 20 aa, Homo sapiens
  MISC_FEATURE: PLP178-197
NTWTTCQSIAFPSKTSASIG

SEQ ID NO: 199 - protein, 20 aa, Homo sapiens
  MISC_FEATURE: PLP190-209
SKTSASIGSLCADARMYGVL

SEQ ID NO: 200 - protein, 20 aa, Homo sapiens
  MISC_FEATURE: MOG11-30
PIRALVGDEVELPCRISPGK

SEQ ID NO: 201 - protein, 20 aa, Homo sapiens
  MISC_FEATURE: MOG21-40
ELPCRISPGKNATGMEVGWY

SEQ ID NO: 202 - protein, 23 aa, Homo sapiens
  MISC_FEATURE: MOG64-86
EYRGRTELLKDAIGEGKVTLRIR

SEQ ID NO: 203 - protein, 86 aa, Homo sapiens
  MISC_FEATURE: Proinsulin 1-86
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 204 - protein, 70 aa, Homo sapiens
  MISC_FEATURE: proinsulin 1-70
FVNQHLCGSHLVEALYLVCGERGFFYTPKTRREAEDLQVGQVELGGGPGAGSLQPLALEGSLQKRGIVEQ

SEQ ID NO: 205 - protein, 62 aa, Homo sapiens
  MISC_FEATURE: Proinsulin 9-70
SHLVEALYLVCGERGFFYTPKTRREAEDLQVGQVELGGGPGAGSLQPLALEGSLQKRGIVEQ

SEQ ID NO: 206 - protein, 30 aa, Homo sapiens
  MISC_FEATURE: Proinsulin 9-38
SHLVEALYLVCGERGFFYTPKTRREAEDLQ

SEQ ID NO: 207 - protein, 38 aa, Homo sapiens
  MISC_FEATURE: Proinsulin 1-38
FVNQHLCGSHLVEALYLVCGERGFFYTPKTRREAEDLQ

SEQ ID NO: 208 - protein, 15 aa, Homo sapiens
  MISC_FEATURE: Prinsulin 9-23
SHLVEALYLVCGERG

SEQ ID NO: 209 - protein, 27 aa, Homo sapiens
  MISC_FEATURE: Proinsulin C13-A6
GGGPGAGSLQPLALEGSLQKRGIVEQC

SEQ ID NO: 210 - protein, 11 aa, Homo sapiens
  MISC_FEATURE: Proinsulin C24-A1
LALEGSLQKRG

SEQ ID NO: 211 - protein, 18 aa, Homo sapiens
  MISC_FEATURE: Proinsulin C19-A3
GSLQPLALEGSLQKRGIV

SEQ ID NO: 212 - protein, 20 aa, Homo sapiens
  MISC_FEATURE: Proinsulin C13-32
GGGPGAGSLQPLALEGSLQK

SEQ ID NO: 213 - protein, 28 aa, Homo sapiens
  MISC_FEATURE: Proinsulin B9-C4
SHLVEALYLVCGERGFFYTPKTRREAED

SEQ ID NO: 214 - protein, 17 aa, Homo sapiens
  MISC_FEATURE: Proinsulin C22-A5
QPLALEGSLQKRGIVEQ

SEQ ID NO: 215 - protein, 15 aa, Homo sapiens
  MISC_FEATURE: Gliadin 15-mer fragment
ELQPFPQPELPYPQP

SEQ ID NO: 216 - protein, 140 aa, Homo sapiens
  MISC_FEATURE: Alpha synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 217 - protein, 115 aa, Homo sapiens
  MISC_FEATURE: 3-103M1-CDRH1_1
QVQLVQSGAEVKKPGASVKVSCTASGFNIRATYILWVRQAPGQGLEWMGRIDPANANSRYDPKFQGRVTITADTSTNTAYMELSSLRSEDTAVYYCVDGYMFAYWGQGTLVTVSS

SEQ ID NO: 218 - protein, 115 aa, Homo sapiens
  MISC_FEATURE: 3-103M1-CDRH1_3
QVQLVQSGAEVKKPGASVKVSCTASGFNIRNIYILWVRQAPGQGLEWMGRIDPANANSRYDPKFQGRVTITADTSTNTAYMELSSLRSEDTAVYYCVDGYMFAYWGQGTLVTVSS

SEQ ID NO: 219 - protein, 115 aa, Homo sapiens
  MISC_FEATURE: 3-103M1-CDRH1_4
QVQLVQSGAEVKKPGASVKVSCTASGFNIKHTYILWVRQAPGQGLEWMGRIDPANANSRYDPKFQGRVTITADTSTNTAYMELSSLRSEDTAVYYCVDGYMFAYWGQGTLVTVSS

SEQ ID NO: 220 - protein, 115 aa, Homo sapiens
  MISC_FEATURE: 3-103M1-CDRH1_8
QVQLVQSGAEVKKPGASVKVSCTASGFNIVHTYILWVRQAPGQGLEWMGRIDPANANSRYDPKFQGRVTITADTSTNTAYMELSSLRSEDTAVYYCVDGYMFAYWGQGTLVTVSS

SEQ ID NO: 221 - protein, 115 aa, Homo sapiens
  MISC_FEATURE: 3-103M1-CDRH1_13
QVQLVQSGAEVKKPGASVKVSCTASGFNIRNVFILWVRQAPGQGLEWMGRIDPANANSRYDPKFQGRVTITADTSTNTAYMELSSLRSEDTAVYYCVDGYMFAYWGQGTLVTVSS

SEQ ID NO: 222 - protein, 115 aa, Homo sapiens
  MISC_FEATURE: 3-103M1-CDRH1_14
QVQLVQSGAEVKKPGASVKVSCTASGFNIRNIYLLWVRQAPGQGLEWMGRIDPANANSRYDPKFQGRVTITADTSTNTAYMELSSLRSEDTAVYYCVDGYMFAYWGQGTLVTVSS

SEQ ID NO: 223 - protein, 115 aa, Homo sapiens
  MISC_FEATURE: 3-103M1-CDRH1_15
QVQLVQSGAEVKKPGASVKVSCTASGFNIRKTYILWVRQAPGQGLEWMGRIDPANANSRYDPKFQGRVTITADTSTNTAYMELSSLRSEDTAVYYCVDGYMFAYWGQGTLVTVSS

SEQ ID NO: 224 - protein, 115 aa, Homo sapiens
  MISC_FEATURE: 3-103M1-CDRH1_19
QVQLVQSGAEVKKPGASVKVSCTASGFNIRHSYILWVRQAPGQGLEWMGRIDPANANSRYDPKFQGRVTITADTSTNTAYMELSSLRSEDTAVYYCVDGYMFAYWGQGTLVTVSS

SEQ ID NO: 225 - protein, 115 aa, Homo sapiens
  MISC_FEATURE: 3-103M1-CDRH1_22
QVQLVQSGAEVKKPGASVKVSCTASGFNILNVYILWVRQAPGQGLEWMGRIDPANANSRYDPKFQGRVTITADTSTNTAYMELSSLRSEDTAVYYCVDGYMFAYWGQGTLVTVSS

SEQ ID NO: 226 - protein, 115 aa, Homo sapiens
  MISC_FEATURE: 3-103M1-CDRH1_22
QVQLVQSGAEVKKPGASVKVSCTASGFNIKATYILWVRQAPGQGLEWMGRIDPANANSRYDPKFQGRVTITADTSTNTAYMELSSLRSEDTAVYYCVDGYMFAYWGQGTLVTVSS

SEQ ID NO: 227 - protein, 115 aa, Homo sapiens
  MISC_FEATURE: 3-103M1-CDRH1_23
QVQLVQSGAEVKKPGASVKVSCTASGFNIRMTYILWVRQAPGQGLEWMGRIDPANANSRYDPKFQGRVTITADTSTNTAYMELSSLRSEDTAVYYCVDGYMFAYWGQGTLVTVSS

SEQ ID NO: 228 - protein, 115 aa, Homo sapiens
  MISC_FEATURE: 3-103M1-CDRH1_24
QVQLVQSGAEVKKPGASVKVSCTASGFNIKTVYILWVRQAPGQGLEWMGRIDPANANSRYDPKFQGRVTITADTSTNTAYMELSSLRSEDTAVYYCVDGYMFAYWGQGTLVTVSS

SEQ ID NO: 229 - protein, 115 aa, Homo sapiens
  MISC_FEATURE: 3-103M1-CDRH1_25
QVQLVQSGAEVKKPGASVKVSCTASGFNIKHVYILWVRQAPGQGLEWMGRIDPANANSRYDPKFQGRVTITADTSTNTAYMELSSLRSEDTAVYYCVDGYMFAYWGQGTLVTVSS

SEQ ID NO: 230 - protein, 115 aa, Homo sapiens
  MISC_FEATURE: 3-103M1-CDRH1_26
QVQLVQSGAEVKKPGASVKVSCTASGFNIRNIYMLWVRQAPGQGLEWMGRIDPANANSRYDPKFQGRVTITADTSTNTAYMELSSLRSEDTAVYYCVDGYMFAYWGQGTLVTVSS

SEQ ID NO: 231 - protein, 115 aa, Homo sapiens
  misc_feature: 3-103M1-CDRH1_49
QVQLVQSGAEVKKPGASVKVSCTASGFNIKDTYILWVRQAPGQGLEWMGRIDPANANSRYDPKFQGRVTITADTSTNTAYMELSSLRSEDTAVYYCVDGYMFAYWGQGTLVTVSS

SEQ ID NO: 232 - protein, 115 aa, Homo sapiens
  MISC_FEATURE: 3-103M1-CDRH1_58
QVQLVQSGAEVKKPGASVKVSCTASGFNIINSYILWVRQAPGQGLEWMGRIDPANANSRYDPKFQGRVTITADTSTNTAYMELSSLRSEDTAVYYCVDGYMFAYWGQGTLVTVSS

SEQ ID NO: 233 - protein, 115 aa, Homo sapiens
  MISC_FEATURE: 3-103M1-CDRH1_64
QVQLVQSGAEVKKPGASVKVSCTASGFNIQHTYILWVRQAPGQGLEWMGRIDPANANSRYDPKFQGRVTITADTSTNTAYMELSSLRSEDTAVYYCVDGYMFAYWGQGTLVTVSS

SEQ ID NO: 234 - protein, 115 aa, Homo sapiens
  MISC_FEATURE: 3-103M1-M10 WT VH
QVQLVQSGAEVKKPGASVKVSCTASGFNIKDTYMLWVRQAPGQGLEWMGRIDPANANSRYDPKFQGRVTITADTSTNTAYMELSSLRSEDTAVYYCVDGYMFAYWGQGTLVTVSS

SEQ ID NO: 235 - protein, 112 aa, Homo sapiens
  MISC_FEATURE: 3-103M1-CDRL1a_4
DVVMTQTPLSLSVTPGQPASISCKSSQSLLFRNNKTYLHWLLQKPGQSPQRLIYLVSKLDSGVPDRFSGSGSGTDFTLKISRVEAEDVGVYYCVQGTHFPLTFGQGTKLEIK

SEQ ID NO: 236 - protein, 112 aa, Homo sapiens
  MISC_FEATURE: 3-103M1-CDRL1a_5
DVVMTQTPLSLSVTPGQPASISCKSSQSLLFRNSKTYLHWLLQKPGQSPQRLIYLVSKLDSGVPDRFSGSGSGTDFTLKISRVEAEDVGVYYCVQGTHFPLTFGQGTKLEIK

SEQ ID NO: 237 - protein, 112 aa, Homo sapiens
  MISC_FEATURE: 3-103M1-CDRL1a_6
DVVMTQTPLSLSVTPGQPASISCKSSQSLLFKNGKTYLHWLLQKPGQSPQRLIYLVSKLDSGVPDRFSGSGSGTDFTLKISRVEAEDVGVYYCVQGTHFPLTFGQGTKLEIK

SEQ ID NO: 238 - protein, 112 aa, Homo sapiens
  MISC_FEATURE: 3-103M1-CDRL1a_10
DVVMTQTPLSLSVTPGQPASISCKSSQSLLFRNAKTYLHWLLQKPGQSPQRLIYLVSKLDSGVPDRFSGSGSGTDFTLKISRVEAEDVGVYYCVQGTHFPLTFGQGTKLEIK

SEQ ID NO: 239 - protein, 112 aa, Homo sapiens
  MISC_FEATURE: 3-103M1-CDRL1a_21
DVVMTQTPLSLSVTPGQPASISCKSSQSLLFRTGKTYLHWLLQKPGQSPQRLIYLVSKLDSGVPDRFSGSGSGTDFTLKISRVEAEDVGVYYCVQGTHFPLTFGQGTKLEIK

SEQ ID NO: 240 - protein, 112 aa, Homo sapiens
  MISC_FEATURE: 3-103M1-CDRL1a_24
DVVMTQTPLSLSVTPGQPASISCKSSQSLLFKNDKTYLHWLLQKPGQSPQRLIYLVSKLDSGVPDRFSGSGSGTDFTLKISRVEAEDVGVYYCVQGTHFPLTFGQGTKLEIK

SEQ ID NO: 241 - protein, 112 aa, Homo sapiens
  MISC_FEATURE: 3-103M1-CDRL1a_32
DVVMTQTPLSLSVTPGQPASISCKSSQSLLYKNGKTYLHWLLQKPGQSPQRLIYLVSKLDSGVPDRFSGSGSGTDFTLKISRVEAEDVGVYYCVQGTHFPLTFGQGTKLEIK

SEQ ID NO: 242 - protein, 112 aa, Homo sapiens
  MISC_FEATURE: 3-103M1-M10 WT VK
DVVMTQTPLSLSVTPGQPASISCKSSQSLLYSNGKTYLHWLLQKPGQSPQRLIYLVSKLDSGVPDRFSGSGSGTDFTLKISRVEAEDVGVYYCVQGTHFPLTFGQGTKLEIK

SEQ ID NO: 243 - protein, 112 aa, Homo sapiens
  MISC_FEATURE: 3-103M1-CDRL2_11
DVVMTQTPLSLSVTPGQPASISCKSSQSLLYSNGKTYLHWLLQKPGQSPQRLIYLLSRTSSGVPDRFSGSGSGTDFTLKISRVEAEDVGVYYCVQGTHFPLTFGQGTKLEIK

SEQ ID NO: 244 - protein, 112 aa, Homo sapiens
  MISC_FEATURE: 3-103M1-CDRL2_12
DVVMTQTPLSLSVTPGQPASISCKSSQSLLYSNGKTYLHWLLQKPGQSPQRLIYLNTRTSSGVPDRFSGSGSGTDFTLKISRVEAEDVGVYYCVQGTHFPLTFGQGTKLEIK

SEQ ID NO: 245 - protein, 112 aa, Homo sapiens
  MISC_FEATURE: 3-103M1-CDRL2_17
DVVMTQTPLSLSVTPGQPASISCKSSQSLLYSNGKTYLHWLLQKPGQSPQRLIYLNTRPSSGVPDRFSGSGSGTDFTLKISRVEAEDVGVYYCVQGTHFPLTFGQGTKLEIK

SEQ ID NO: 246 - protein, 112 aa, Homo sapiens
  MISC_FEATURE: 3-103M1-CDRL2_18
DVVMTQTPLSLSVTPGQPASISCKSSQSLLYSNGKTYLHWLLQKPGQSPQRLIYLLNRLHSGVPDRFSGSGSGTDFTLKISRVEAEDVGVYYCVQGTHFPLTFGQGTKLEIK

SEQ ID NO: 247 - protein, 112 aa, Homo sapiens
  MISC_FEATURE: 3-103M1-CDRL2_19
DVVMTQTPLSLSVTPGQPASISCKSSQSLLYSNGKTYLHWLLQKPGQSPQRLIYLNTRLASGVPDRFSGSGSGTDFTLKISRVEAEDVGVYYCVQGTHFPLTFGQGTKLEIK

SEQ ID NO: 248 - protein, 111 aa, Homo sapiens
  MISC_FEATURE: 3-103M1-CDRL2_27
VVMTQTPLSLSVTPGQPASISCKSSQSLLYSNGKTYLHWLLQKPGQSPQRLIYLNSRLSSGVPDRFSGSGSGTDFTLKISRVEAEDVGVYYCVQGTHFPLTFGQGTKLEIK

SEQ ID NO: 249 - protein, 112 aa, Homo sapiens
  MISC_FEATURE: 3-103M1-CDRL2_28
DVVMTQTPLSLSVTPGQPASISCKSSQSLLYSNGKTYLHWLLQKPGQSPQRLIYLLSRVSSGVPDRFSGSGSGTDFTLKISRVEAEDVGVYYCVQGTHFPLTFGQGTKLEIK

SEQ ID NO: 250 - protein, 112 aa, Homo sapiens
  MISC_FEATURE: 3-103M1-CDRL2_29
DVVMTQTPLSLSVTPGQPASISCKSSQSLLYSNGKTYLHWLLQKPGQSPQRLIYLLNRVSSGVPDRFSGSGSGTDFTLKISRVEAEDVGVYYCVQGTHFPLTFGQGTKLEIK

SEQ ID NO: 251 - protein, 112 aa, Homo sapiens
  MISC_FEATURE: 3-103M1-CDRL2_32
DVVMTQTPLSLSVTPGQPASISCKSSQSLLYSNGKTYLHWLLQKPGQSPQRLIYLLNRLSSGVPDRFSGSGSGTDFTLKISRVEAEDVGVYYCVQGTHFPLTFGQGTKLEIK

SEQ ID NO: 252 - protein, 112 aa, Homo sapiens
  MISC_FEATURE: 3-103M1-CDRL2_33
DVVMTQTPLSLSVTPGQPASISCKSSQSLLYSNGKTYLHWLLQKPGQSPQRLIYLNSRLHSGVPDRFSGSGSGTDFTLKISRVEAEDVGVYYCVQGTHFPLTFGQGTKLEIK

SEQ ID NO: 253 - protein, 112 aa, Homo sapiens
  MISC_FEATURE: 3-103M1-CDRL2_34
DVVMTQTPLSLSVTPGQPASISCKSSQSLLYSNGKTYLHWLLQKPGQSPQRLIYLSSRLSSGVPDRFSGSGSGTDFTLKISRVEAEDVGVYYCVQGTHFPLTFGQGTKLEIK

SEQ ID NO: 254 - protein, 112 aa, Homo sapiens
  MISC_FEATURE: 3-103M1-CDRL2_35
DVVMTQTPLSLSVTPGQPASISCKSSQSLLYSNGKTYLHWLLQKPGQSPQRLIYLSSRVSSGVPDRFSGSGSGTDFTLKISRVEAEDVGVYYCVQGTHFPLTFGQGTKLEIK

SEQ ID NO: 255 - protein, 112 aa, Homo sapiens
  MISC_FEATURE: 3-103M1-CDRL2_37
DVVMTQTPLSLSVTPGQPASISCKSSQSLLYSNGKTYLHWLLQKPGQSPQRLIYLSNRLHSGVPDRFSGSGSGTDFTLKISRVEAEDVGVYYCVQGTHFPLTFGQGTKLEIK

SEQ ID NO: 256 - protein, 112 aa, Homo sapiens
  MISC_FEATURE: 3-103M1-CDRL2_38
DVVMTQTPLSLSVTPGQPASISCKSSQSLLYSNGKTYLHWLLQKPGQSPQRLIYLNTRVSSGVPDRFSGSGSGTDFTLKISRVEAEDVGVYYCVQGTHFPLTFGQGTKLEIK

SEQ ID NO: 257 - protein, 112 aa, Homo sapiens
  MISC_FEATURE: 3-103M1-CDRL2_39
DVVMTQTPLSLSVTPGQPASISCKSSQSLLYSNGKTYLHWLLQKPGQSPQRLIYLSNRVSSGVPDRFSGSGSGTDFTLKISRVEAEDVGVYYCVQGTHFPLTFGQGTKLEIK

SEQ ID NO: 258 - protein, 112 aa, Homo sapiens
  MISC_FEATURE: 3-103M1-CDRL2_40
DVVMTQTPLSLSVTPGQPASISCKSSQSLLYSNGKTYLHWLLQKPGQSPQRLIYLHSRLSSGVPDRFSGSGSGTDFTLKISRVEAEDVGVYYCVQGTHFPLTFGQGTKLEIK

SEQ ID NO: 259 - protein, 112 aa, Homo sapiens
  MISC_FEATURE: 3-103M1-CDRL2_44
DVVMTQTPLSLSVTPGQPASISCKSSQSLLYSNGKTYLHWLLQKPGQSPQRLIYLSSRLASGVPDRFSGSGSGTDFTLKISRVEAEDVGVYYCVQGTHFPLTFGQGTKLEIK

SEQ ID NO: 260 - protein, 112 aa, Homo sapiens
  MISC_FEATURE: 3-103M1-CDRL2_48
DVVMTQTPLSLSVTPGQPASISCKSSQSLLYSNGKTYLHWLLQKPGQSPQRLIYLFNRVNSGVPDRFSGSGSGTDFTLKISRVEAEDVGVYYCVQGTHFPLTFGQGTKLEIK

SEQ ID NO: 261 - protein, 112 aa, Homo sapiens
  MISC_FEATURE: 3-103M1-CDRL2_49
DVVMTQTPLSLSVTPGQPASISCKSSQSLLYSNGKTYLHWLLQKPGQSPQRLIYLLNRMSSGVPDRFSGSGSGTDFTLKISRVEAEDVGVYYCVQGTHFPLTFGQGTKLEIK

SEQ ID NO: 262 - protein, 112 aa, Homo sapiens
  MISC_FEATURE: 3-103M1-CDRL2_5
DVVMTQTPLSLSVTPGQPASISCKSSQSLLYSNGKTYLHWLLQKPGQSPQRLIYLLNRISSGVPDRFSGSGSGTDFTLKISRVEAEDVGVYYCVQGTHFPLTFGQGTKLEIK

SEQ ID NO: 263 - protein, 112 aa, Homo sapiens
  MISC_FEATURE: 3-103M1-CDRL2_54
DVVMTQTPLSLSVTPGQPASISCKSSQSLLYSNGKTYLHWLLQKPGQSPQRLIYLLSHPHSGVPDRFSGSGSGTDFTLKISRVEAEDVGVYYCVQGTHFPLTFGQGTKLEIK

SEQ ID NO: 264 - protein, 112 aa, Homo sapiens; identical to SEQ ID NO: 242
  MISC_FEATURE: 3-103M1-M10 WT VK

SEQ ID NO: 265 - protein, 112 aa, Homo sapiens; identical to SEQ ID NO: 242
  MISC_FEATURE: 3-103M1 WT Variable Light Chain

SEQ ID NO: 266 - protein, 107 aa, Homo sapiens [sequence omitted]

SEQ ID NO: 267 - protein, 115 aa, Homo sapiens; identical to SEQ ID NO: 234
  MISC_FEATURE: 3-103M1 -M10 WT Variable Heavy

SEQ ID NO: 268 - protein, 103 aa, Homo sapiens
  MISC_FEATURE: 3-103M1 -M10 WT Constant Heavy Chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 269 - protein, 6 aa, Homo sapiens
  MISC_FEATURE: 3-103M1-CDRH1_1
RATYIL

SEQ ID NO: 270 - protein, 6 aa, Homo sapiens
  MISC_FEATURE: 3-103M1-CDRH1_3
RNIYIL

SEQ ID NO: 271 - protein, 6 aa, Homo sapiens
  MISC_FEATURE: 3-103M1-CDRH1_4
KHTYIL

SEQ ID NO: 272 - protein, 6 aa, Homo sapiens
  MISC_FEATURE: 3-103M1-CDRH1_8
VHTYIL

SEQ ID NO: 273 - protein, 6 aa, Homo sapiens
  MISC_FEATURE: 3-103M1-CDRH1_13
RNVFIL

SEQ ID NO: 274 - protein, 6 aa, Homo sapiens
  MISC_FEATURE: 3-103M1-CDRH1_14
RNIYLL

SEQ ID NO: 275 - protein, 6 aa, Homo sapiens
  MISC_FEATURE: 3-103M1-CDRH1_15
RKTYIL

SEQ ID NO: 276 - protein, 6 aa, Homo sapiens
  MISC_FEATURE: 3-103M1-CDRH1_21
LNVYIL

SEQ ID NO: 277 - protein, 6 aa, Homo sapiens
  MISC_FEATURE: 3-103M1-CDRH1_22
KATYIL

SEQ ID NO: 278 - protein, 6 aa, Homo sapiens
  MISC_FEATURE: 3-103M1-CDRH1_23
RMTYIL

SEQ ID NO: 279 - protein, 6 aa, Homo sapiens
  MISC_FEATURE: 3-103M1-CDRH1_24
KTVYIL

SEQ ID NO: 280 - protein, 6 aa, Homo sapiens
  MISC_FEATURE: 3-103M1-CDRH1_25
KHVYIL

SEQ ID NO: 281 - protein, 6 aa, Homo sapiens
  MISC_FEATURE: 3-103M1-CDRH1_26
RNIYML

SEQ ID NO: 282 - protein, 6 aa, Homo sapiens
  MISC_FEATURE: 3-103M1-CDRH1_49
KDTYIL

SEQ ID NO: 283 - protein, 6 aa, Homo sapiens
  MISC_FEATURE: 3-103M1-CDRH1_58
INSYIL

SEQ ID NO: 284 - protein, 6 aa, Homo sapiens
  MISC_FEATURE: 3-103M1-CDRH1_64
QHTYIL

SEQ ID NO: 285 - protein, 6 aa, Homo sapiens
  MISC_FEATURE: 3-103M1-CDRH1_19
RHSYIL

SEQ ID NO: 286 - protein, 6 aa, Homo sapiens
  MISC_FEATURE: CDRH1 Parent Sequence
KDTYML

SEQ ID NO: 287 - protein, 5 aa, Homo sapiens
  MISC_FEATURE: 3-103M1-CDRL1a_4
FRNNK

SEQ ID NO: 288 - protein, 5 aa, Homo sapiens
  MISC_FEATURE: 3-103M1-CDRL1a_5
FRNSK

SEQ ID NO: 289 - protein, 5 aa, Homo sapiens
  MISC_FEATURE: 3-103M1-CDRL1a_6
FKNGK

SEQ ID NO: 290 - protein, 5 aa, Homo sapiens
  MISC_FEATURE: 3-103M1-CDRL1a_10
FRNAK

SEQ ID NO: 291 - protein, 5 aa, Homo sapiens
  MISC_FEATURE: 3-103M1-CDRL1a_21
FRTGK

SEQ ID NO: 292 - protein, 5 aa, Homo sapiens
  MISC_FEATURE: 3-103M1-CDRL1a_24
FKNDK

SEQ ID NO: 293 - protein, 5 aa, Homo sapiens
  MISC_FEATURE: 3-103M1-CDRL1a_32
YKNGK

SEQ ID NO: 294 - protein, 6 aa, Homo sapiens
  MISC_FEATURE: CDRL1a parent sequence
YSNGKT

SEQ ID NO: 295 - protein, 5 aa, Homo sapiens
  MISC_FEATURE: 3-103M1-CDRL2_11
LSRTS

SEQ ID NO: 296 - protein, 5 aa, Homo sapiens
  MISC_FEATURE: 3-103M1-CDRL2_12
NTRTS

SEQ ID NO: 297 - protein, 5 aa, Homo sapiens
  MISC_FEATURE: 3-103M1-CDRL2_17
NTRPS

SEQ ID NO: 298 - protein, 5 aa, Homo sapiens
  MISC_FEATURE: 3-103M1-CDRL2_18
LNRLH

SEQ ID NO: 299 - protein, 5 aa, Homo sapiens
  MISC_FEATURE: 3-103M1-CDRL2_19
NTRLA

SEQ ID NO: 300 - protein, 5 aa, Homo sapiens
  MISC_FEATURE: 3-103M1-CDRL2_27
NSRLS

SEQ ID NO: 301 - protein, 5 aa, Homo sapiens
  MISC_FEATURE: 3-103M1-CDRL2_28
LSRVS

SEQ ID NO: 302 - protein, 5 aa, Homo sapiens
  MISC_FEATURE: 3-103M1-CDRL2_29
LNRVS

SEQ ID NO: 303 - protein, 5 aa, Homo sapiens
  MISC_FEATURE: 3-103M1-CDRL2_32
LNRLS

SEQ ID NO: 304 - protein, 5 aa, Homo sapiens
  MISC_FEATURE: 3-103M1-CDRL2_33
NSRLH

SEQ ID NO: 305 - protein, 5 aa, Homo sapiens
  MISC_FEATURE: 3-103M1-CDRL2_34
SSRLS

SEQ ID NO: 306 - protein, 5 aa, Homo sapiens
  MISC_FEATURE: 3-103M1-CDRL2_35
SSRVS

SEQ ID NO: 307 - protein, 5 aa, Homo sapiens
  MISC_FEATURE: 3-103M1-CDRL2_37
SNRLH

SEQ ID NO: 308 - protein, 5 aa, Homo sapiens
  MISC_FEATURE: 3-103M1-CDRL2_38
NTRVS

SEQ ID NO: 309 - protein, 5 aa, Homo sapiens
  MISC_FEATURE: 3-103M1-CDRL2_39
SNRVS

SEQ ID NO: 310 - protein, 5 aa, Homo sapiens
  MISC_FEATURE: 3-103M1-CDRL2_40
HSRLS

SEQ ID NO: 311 - protein, 5 aa, Homo sapiens
  MISC_FEATURE: 3-103M1-CDRL2_44
SSRLA

SEQ ID NO: 312 - protein, 5 aa, Homo sapiens
  MISC_FEATURE: 3-103M1-CDRL2_48
FNRVN

SEQ ID NO: 313 - protein, 5 aa, Homo sapiens
  MISC_FEATURE: 3-103M1-CDRL2_49
LNRMS

SEQ ID NO: 314 - protein, 5 aa, Homo sapiens
  MISC_FEATURE: 3-103M1-CDRL2_5
LNRIS

SEQ ID NO: 315 - protein, 5 aa, Homo sapiens
  MISC_FEATURE: 3-103M1-CDRL2_54
LSHPH

SEQ ID NO: 316 - protein, 5 aa, Homo sapiens
  MISC_FEATURE: CDRL2 Parent Sequence
VSKLD